SEQUENCE LISTING (145 sequences)

SEQ ID NO: 1 - protein, 123 aa, synthetic construct
QVQLVQSGSELKKPGASVKVSCKASGYTFTTYSLNWVRQAPGQGLEWMGWINTYTGNPTYAQGFTGRFVFFLDTSVSTAYLQISSLKAEDTAVYYCARETYYYDSSGYPFDPWGQGTLVTVSS

SEQ ID NO: 2 - protein, 123 aa, synthetic construct
QVQLVQSGSELKKPGASVKVSCKASGYTFTSYAMNWVRQAPGQGLEWMGWINTNTGNPTYAQGFTGRFVFSLDTSVSTAYLQISSLKAEDTAVYYCARETYYYDRGGYPFDPWGQGTLVTVSS

SEQ ID NO: 3 - protein, 123 aa, synthetic construct
QVQLMQSGSELKKPGASVKVSCKASGYTFTTYSMNWVRQAPGQGLEWMGWINTNTGNPTYAQDFTGRFVFSLDTSVSTAYLQISSLKAEDTAVYYCARETYYYDSSGYPFDPWGQGTLVTVSS

SEQ ID NO: 4 - protein, 123 aa, synthetic construct
QVQLVQSGSELKKPGASVKVSCKASGYTFTDYAMNWVRQVPGQGLEWMGWINTYTGNPTYVQGFTGRFVFSLDTSVSTAYLQISSLKAEDTAVYYCARETYYYDSSGFPFDPWGQGTLVTVSS

SEQ ID NO: 5 - protein, 123 aa, synthetic construct
QVQLVQSGSELKKPGASVKVSCKASGYTFTNYAMNWVRQAPGQGLEWMGWINTNTGNPTYAQGFTGRFVFPLDTSVSTTYLQISSLKAEDTAVYYCARETYYYQSSGYLFDPWGQGTLVTVSS

SEQ ID NO: 6 - protein, 123 aa, synthetic construct
QVQLVQSGSELKKPGASVKVSCKASGYTFTSFGMSWVRQAPGQGLEWMGWINTNTGNPTYAQGFTGRFVFSLDTSVSTAYLQINSLKAEDTAVYYCARESYYYDRNDYPFDPWGQGTLVTVSS

SEQ ID NO: 7 - protein, 123 aa, synthetic construct
QVQLVQSGSELKKPGASVKVSCKASGYTFTTYSMNWVRQAPGQGLEWMGWINTNTGNPTYAQGFTGRFVFSLDTSVSTAYLQISSLNTEDTAVYYCARETYYYDVGGYPFDPWGQGTLVTVSS

SEQ ID NO: 8 - protein, 123 aa, synthetic construct
QVQLVQSGSELEKPGASVKVSCKASGYTFISYAMNWVRQAPGQGLEWMGWINTYTGNPTYAQGFTGRFVFSLDTSVSTAYLQISSLKAEDTAVYYCARETYYYDSGGYPFDPWGQGTLVTVSS

SEQ ID NO: 9 - protein, 123 aa, synthetic construct
QVQLVQSGSELKKPGASVKVSCKASGYTFTSYAVNWVRQAPGQGLEWMGWINTYTGNPTYAQGFTGRFVFSSDTSVNTAYLQISSLKAEDTAVYYCARETYFYDSSGYPFDPWGQGTLVTVSS

SEQ ID NO: 10 - protein, 123 aa, synthetic construct
QVQVVQSGSEVKKPGASVKVSCKASGYTFTTYSMNWVRQAPGQGLEWMGWINTYTGDPTYVQGFTGRFVFSLDTSVSTAYLQISSLKAEDTAVYYCARETYFYDRGGYPFDPWGQGTLVTVSS

SEQ ID NO: 11 - protein, 123 aa, synthetic construct
QVQLVQSGSELKKPGASVKVSCKASGYTFTDYAMNWVRQAPGQGLEWMGWINTYTGNPTYVQGFTGRFVFSLDTSVSTAYLQISSLKAEDTAVYYCARETYYYDSSAYPFDPWGQGTLVTVSS

SEQ ID NO: 12 - protein, 123 aa, synthetic construct
QVQLVQSGSELEKPGASVKVSCKASGYTFTTYSMNWVRQAPGQGLEWMGWINTYTGSPTYAQGFTGRFVFSLDTSVSTAYLQISSLKAEDTAIYYCARETFYFDSGGYPFDPWGQGTLVTVSS

SEQ ID NO: 13 - protein, 123 aa, synthetic construct
QVQLVQSGSELKKPGASVKVSCKASGYTFTTYSMNWVRQAPGQGLEWMGWINTYTGDPTYAQGFTGRFVFSLDTSVSTAYLQISSLKAEDTAVYYCARETYFYDRGGYPFDPWGQGTLVTVSS

SEQ ID NO: 14 - protein, 123 aa, synthetic construct
QVQLVQSGSELKKPGASVKVSCKASGYTFTTYAINWVRQAPGQGLEWMGWINTNTGNPTYAQGFTGRFVFSLDTSVSTAHLQISSLKAEDTAVYYCARETYYYATSGYPFDPWGQGALVTVSS

SEQ ID NO: 15 - protein, 123 aa, synthetic construct
QVQLVQSGSELKKPGASVKVSCKASGYTFTTYAMNWVRQAPGQGLEWMGWINTYTGDPTYAQGFTGRFVFSLDTSVNTAYLQISSLKAEDTAVYYCARETSYYDRTGYPFDPWGQGTLVTVSS

SEQ ID NO: 16 - protein, 123 aa, synthetic construct
QVQLVQSGSELKKPGASVKVSCKASGYTFTTYAMNWVRQAPGQGLEWMGWINTNTGNPTYAQGFTGRFVFSLDTSDSTAFLQISSLKAEDTAVYYCARETYYYDSSGYPFDPWGQGTLVTVSS

SEQ ID NO: 17 - protein, 122 aa, synthetic construct
VELVQSGSELKKPGASVKVSCKASGYTFTTYSMNWVRQAPGQGLEWMGWINTNTGTPTYAQGFTGRFVFSLDTSVSTAYLQISSLKAEDTAVYYCARETYYYDSSGYPFDPWGQGTLVTVSS

SEQ ID NO: 18 - protein, 123 aa, synthetic construct
QVQLVQSGSELKKPGASVKVSCKASGYTFTTYSMNWVRQAPGQGLEWMGWINTNTGTPTYAQGFTGRFVFSLDTSVSTAYLQISSLKAEDTAVYYCARETYYYGSSGYPFAPWGQGTLVTVSS

SEQ ID NO: 19 - protein, 123 aa, synthetic construct
QVQLVQSGSELKKPGASVKVSCKASGYTFTTYSMNWVRQAPGQGLEWMGWINTYTGNPTYAQGFTGRFVFSLDTSVSTAYLQISSLTTEDTAVYYCARETYYYESSGYPFDPWGQGTLVTVSS

SEQ ID NO: 20 - protein, 123 aa, synthetic construct
QVQLVQSGSELEKPGASVKVSCKASGYTFTTYSMNWVRQAPGQGLEWMGWINTYTGSPTYAQGFTGRFVFSLDTSVSTAYLQISSLKAEDTAVYYCARETYYFDSGDYPFDPWGQGTLVTVSS

SEQ ID NO: 21 - protein, 123 aa, synthetic construct
QVQLVQSGSELEKPGASVKVSCKASGYTFTTYSMNWVRQAPGQGLEWMGWINTYTGSPTYAQGFTGRFVFSLDTSVSTAYLQISSLKAEDTAVYYCARETYYFDSGGYPFDPWGQGTLVTVSS

SEQ ID NO: 22 - protein, 123 aa, synthetic construct
QVQLVQSGSELKKPGASVKVSCKASGYTFTDYAMNWVRQAPGQGLEWMGWINTYTGNPTYVQGFTGRFVFSLDTSVSTAYLQISSLKAEDTAVYYCARETYYYDSSGYPFDPWGQGTLVTVSS

SEQ ID NO: 23 - protein, 123 aa, synthetic construct
QVQLVQSGSELKKPGASVKVSCKASGYTFTSYSMNWVRQAPGQGLEWMGWINTYTGDPTYAQGFTGRYVFSLDTSVNTAYLQISSLKAEDTAVYYCARETYYYDRGGYPFDPWGQGTLVTVSS

SEQ ID NO: 24 - protein, 127 aa, synthetic construct
EVQLVETGAEVKKPGASVKVSCKASDYIFTKYDINWVRQAPGQGLEWMGWMSANTGNTGYAQKFQGRVTMTRDTSINTAYMELSSLTSGDTAVYFCARSSLFKTETAPYYHFALDVWGQGTTVTVSS

SEQ ID NO: 25 - protein, 122 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCAKDRHWHWWLDAFDYWGQGTLVTVSS

SEQ ID NO: 26 - protein, 120 aa, synthetic construct
QVQLVQSGSELKKPGASVKISCKASGYDFTNYAMNWVRQAPGHGLEWMGWINANTGDPTYAQGFTGRFVFSLDTSVSTAYLQISSLKAEDSAVYYCTRERFLEWLHFDYWGQGTLVTVSS

SEQ ID NO: 27 - protein, 128 aa, synthetic construct
EVQLVESGGGLVKPGGSLRLSCAASGFTFSKAWMNWVRQAPGKGLEWVGRIKSKTDGGTTDYAAPVKGRFTISRDDSKNTLYLQMNSLKTEDTAVYYCTTASSMITFGGVIVSWFDPWGQGTLVTVSS

SEQ ID NO: 28 - protein, 125 aa, synthetic construct
EVQLVESGGGVVQPGRSLRLSCAASGFTFSNYGMHWVRQAPGKGLEWVAVISYDGSTKYSADSLKGRFTISRDNSKNTLYLQMNSLRADDTAVYYCAKEGWSFDSSGYRSWFDSWGQGTLVTVSS

SEQ ID NO: 29 - protein, 30 aa, synthetic construct
EVQLVETGAEVKKPGASVKVSCKASDYIFT

SEQ ID NO: 30 - protein, 5 aa, synthetic construct
KYDIN

SEQ ID NO: 31 - protein, 14 aa, synthetic construct
WVRQAPGQGLEWMG

SEQ ID NO: 32 - protein, 17 aa, synthetic construct
WMSANTGNTGYAQKFQG

SEQ ID NO: 33 - protein, 32 aa, synthetic construct
RVTMTRDTSINTAYMELSSLTSGDTAVYFCAR

SEQ ID NO: 34 - protein, 18 aa, synthetic construct
SSLFKTETAPYYHFALDV

SEQ ID NO: 35 - protein, 11 aa, synthetic construct
WGQGTTVTVSS

SEQ ID NO: 36 - protein, 30 aa, synthetic construct
QVQLVQSGAEVKKPGASVKVSCKASGYTFT

SEQ ID NO: 37 - protein, 5 aa, synthetic construct
SYGIS

SEQ ID NO: 38 - protein, 17 aa, synthetic construct
WISAYNGNTNYAQKLQG

SEQ ID NO: 39 - protein, 32 aa, synthetic construct
RVTMTTDTSTSTAYMELRSLRSDDTAVYYCAK

SEQ ID NO: 40 - protein, 13 aa, synthetic construct
DRHWHWWLDAFDY

SEQ ID NO: 41 - protein, 11 aa, synthetic construct
WGQGTLVTVSS

SEQ ID NO: 42 - protein, 30 aa, synthetic construct
QVQLVQSGSELKKPGASVKISCKASGYDFT

SEQ ID NO: 43 - protein, 5 aa, synthetic construct
NYAMN

SEQ ID NO: 44 - protein, 14 aa, synthetic construct
WVRQAPGHGLEWMG

SEQ ID NO: 45 - protein, 17 aa, synthetic construct
WINANTGDPTYAQGFTG

SEQ ID NO: 46 - protein, 32 aa, synthetic construct
RFVFSLDTSVSTAYLQISSLKAEDSAVYYCTR

SEQ ID NO: 47 - protein, 11 aa, synthetic construct
ERFLEWLHFDY

SEQ ID NO: 48 - protein, 30 aa, synthetic construct
EVQLVESGGGLVKPGGSLRLSCAASGFTFS

SEQ ID NO: 49 - protein, 5 aa, synthetic construct
KAWMN

SEQ ID NO: 50 - protein, 14 aa, synthetic construct
WVRQAPGKGLEWVG

SEQ ID NO: 51 - protein, 19 aa, synthetic construct
RIKSKTDGGTTDYAAPVKG

SEQ ID NO: 52 - protein, 32 aa, synthetic construct
RFTISRDDSKNTLYLQMNSLKTEDTAVYYCTT

SEQ ID NO: 53 - protein, 17 aa, synthetic construct
ASSMITFGGVIVSWFDP

SEQ ID NO: 54 - protein, 30 aa, synthetic construct
QVQLVQSGSELKKPGASVKVSCKASGYTFT

SEQ ID NO: 55 - protein, 5 aa, synthetic construct
SYSMN

SEQ ID NO: 56 - protein, 17 aa, synthetic construct
WINTYTGDPTYAQGFTG

SEQ ID NO: 57 - protein, 32 aa, synthetic construct
RYVFSLDTSVNTAYLQISSLKAEDTAVYYCAR

SEQ ID NO: 58 - protein, 14 aa, synthetic construct
ETYYYDRGGYPFDP

SEQ ID NO: 59 - protein, 30 aa, synthetic construct
EVQLVESGGGVVQPGRSLRLSCAASGFTFS

SEQ ID NO: 60 - protein, 5 aa, synthetic construct
NYGMH

SEQ ID NO: 61 - protein, 14 aa, synthetic construct
WVRQAPGKGLEWVA

SEQ ID NO: 62 - protein, 17 aa, synthetic construct
VISYDGSTKYSADSLKG

SEQ ID NO: 63 - protein, 32 aa, synthetic construct
RFTISRDNSKNTLYLQMNSLRADDTAVYYCAK

SEQ ID NO: 64 - protein, 16 aa, synthetic construct
EGWSFDSSGYRSWFDS

SEQ ID NO: 65 - protein, 122 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDSYWHWWLGAFDYWGQGTLVTVSS

SEQ ID NO: 66 - protein, 122 aa, synthetic construct
QVQLVQSGAEVKKPGASVKVSCKASGYTFTSYGISWVRQAPGQGLEWMGWISAYNANTNYAQKLQGRVTMTTDTSTSTAYMELRSLRSDDTAVYYCARDSYWHWWLGAFDYWGQGTLVTVSS

SEQ ID NO: 67 - protein, 122 aa, synthetic construct
QVQLVQSGAEVKKPGASVKVSCKASGYTFTSYGISWVRQAPGQGLEWMGWISAYSGNTNYAQKLQGRVTMTTDTSTSTAYMELRSLRSDDTAVYYCARDSYWHWWLGAFDYWGQGTLVTVSS

SEQ ID NO: 68 - protein, 122 aa, synthetic construct
QVQLVQSGAEVKKPGASVKVSCKASGYTFTSYGISWVRQAPGQGLEWMGWISAYNANTNYAQKLQGRVTMTTDTSTSTAYMELRSLRSDDTAVYYCAKDRHWHWWLDAFDYWGQGTLVTVSS

SEQ ID NO: 69 - protein, 122 aa, synthetic construct
QVQLVQSGAEVKKPGASVKVSCKASGYTFTSYGISWVRQAPGQGLEWMGWISAYSGNTNYAQKLQGRVTMTTDTSTSTAYMELRSLRSDDTAVYYCAKDRHWHWWLDAFDYWGQGTLVTVSS

SEQ ID NO: 70 - protein, 122 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARGYLDHWWLGAFDYWGQGTLVTVSS

SEQ ID NO: 71 - protein, 122 aa, synthetic construct
QVQLVQSGAEVKKPGASVKVSCKASGYTFTSYGISWVRQAPGQGLEWMGWISAYNANTNYAQKLQGRVTMTTDTSTSTAYMELRSLRSDDTAVYYCARGYLDHWWLGAFDYWGQGTLVTVSS

SEQ ID NO: 72 - protein, 122 aa, synthetic construct
QVQLVQSGAEVKKPGASVKVSCKASGYTFTSYGISWVRQAPGQGLEWMGWISAYSGNTNYAQKLQGRVTMTTDTSTSTAYMELRSLRSDDTAVYYCARGYLDHWWLGAFDYWGQGTLVTVSS

SEQ ID NO: 73 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattagcagctacttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccaacgttcggccaagggaccaaggtggagatcaaa

SEQ ID NO: 75 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 76 - DNA, 324 bp, synthetic construct
  source 1..324: mol_type = other DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 77 - protein, 107 aa, synthetic construct [sequence omitted]

SEQ ID NO: 78 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 79 - protein, 95 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 80 - DNA, 294 bp, synthetic construct
  source 1..294: mol_type = other DNA
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagagagtt

SEQ ID NO: 81 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 82 - DNA, 45 bp, synthetic construct
  source 1..45: mol_type = other DNA
gagcccaaatcttgtgacaaaactcacacatgcccaccgtgccca

SEQ ID NO: 83 - protein, 15 aa, synthetic construct
EPKSCDKTHTCPPCP

SEQ ID NO: 84 - DNA, 330 bp, synthetic construct
  source 1..330: mol_type = other DNA
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaa

SEQ ID NO: 85 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 86 - DNA, 330 bp, synthetic construct
  source 1..330: mol_type = other DNA
gcacctgaactcggcaggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaa

SEQ ID NO: 87 - protein, 110 aa, synthetic construct
APELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 88 - DNA, 321 bp, synthetic construct
  source 1..321: mol_type = other DNA
gggcagccccgagaaccacaggtgtacaccaagcccccatcccgggaggagatgaccaagaaccaggtcagcctgaagtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 89 - protein, 106 aa, synthetic construct
GQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPG

SEQ ID NO: 90 - DNA, 321 bp, synthetic construct
  source 1..321: mol_type = other DNA
gggcagccccgagaaccacaggtgtacaccgaccccccatcccgggaggagatgaccaagaaccaggtcagcctgacctgcgaggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 91 - protein, 106 aa, synthetic construct
GQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPG

SEQ ID NO: 92 - protein, 17 aa, synthetic construct
  VARIANT 6: note = can be N or S
  VARIANT 7: note = can be A or G
WISAYXXNTNYAQKLQG

SEQ ID NO: 93 - protein, 10 aa, synthetic construct
  VARIANT 1: note = can be D or G
  VARIANT 2: note = can be R, S or Y
  VARIANT 3: note = can be H, L or Y
  VARIANT 4: note = can be D or W
  VARIANT 9: note = can be D or G
XXXXHWWLXA

SEQ ID NO: 94 - protein, 5 aa, synthetic construct
DRHWD

SEQ ID NO: 95 - protein, 11 aa, synthetic construct
  VARIANT 1: note = can be D or G
  VARIANT 2: note = can be R, S or Y
  VARIANT 3: note = can be H, L or Y
  VARIANT 4: note = can be D or W
  VARIANT 9: note = can be D or G
XXXXHWWLXAF

SEQ ID NO: 96 - protein, 12 aa, synthetic construct
  VARIANT 1: note = can be D or G
  VARIANT 2: note = can be R, S or Y
  VARIANT 3: note = can be H, L or Y
  VARIANT 4: note = can be D or W
  VARIANT 9: note = can be D or G
XXXXHWWLXAFD

SEQ ID NO: 97 - protein, 13 aa, synthetic construct
  VARIANT 1: note = can be D or G
  VARIANT 2: note = can be R, S or Y
  VARIANT 3: note = can be H, L or Y
  VARIANT 4: note = can be D or W
  VARIANT 9: note = can be D or G
XXXXHWWLXAFDY

SEQ ID NO: 98 - protein, 10 aa, synthetic construct
DRHWHWWLDA

SEQ ID NO: 99 - protein, 11 aa, synthetic construct
DRHWHWWLDAF

SEQ ID NO: 100 - protein, 12 aa, synthetic construct
DRHWHWWLDAFD

SEQ ID NO: 101 - protein, 6 aa, synthetic construct
QSISSY

SEQ ID NO: 102 - protein, 7 aa, synthetic construct
QQSYSTP

SEQ ID NO: 103 - protein, 5 aa, synthetic construct
TYSMN

SEQ ID NO: 104 - protein, 14 aa, synthetic construct
ETYFYDRGGYPFDP

SEQ ID NO: 105 - protein, 17 aa, synthetic construct
WISAYNANTNYAQKLQG

SEQ ID NO: 106 - protein, 17 aa, synthetic construct
WISAYSGNTNYAQKLQG

SEQ ID NO: 107 - protein, 9 aa, synthetic construct
QQSYSTPPT

SEQ ID NO: 108 - protein, 16 aa, synthetic construct
WINTYTGDPTYAQGFT

SEQ ID NO: 109 - protein, 1210 aa, Homo sapiens [sequence omitted]

SEQ ID NO: 110 - protein, 23 aa, synthetic construct
MRPSGTAGAALLALLAALCPASR

SEQ ID NO: 111 - protein, 622 aa, synthetic construct
ALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPS

SEQ ID NO: 112 - protein, 23 aa, synthetic construct
IATGMVGALLLLLVVALGIGLFM

SEQ ID NO: 113 - protein, 542 aa, synthetic construct
RRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 114 - protein, 378 aa, Homo sapiens
MRPSGTAGAALLALLAALCPASRALEEKKGNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPS

SEQ ID NO: 115 - protein, 23 aa, synthetic construct
MRPSGTAGAALLALLAALCPASR

SEQ ID NO: 116 - protein, 355 aa, synthetic construct
ALEEKKGNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPS

SEQ ID NO: 117 - protein, 1210 aa, synthetic construct
MGPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSEFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDTLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSSQKTKIISNRGENSCKATGQVCHALCSPEGCWGPEPRDCVSCQNVSRGRECVDKCNILEGEPREFVENSECIQCHPECLPQVMNITCTGRGPDNCIQCAHYIDGPHCVKTCPAGVMGENNTLVWKYADAGHVCHLCHPNCTYGCTGPGLEGCARNGPKIPSIATGMLGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 118 - protein, 23 aa, synthetic construct
MGPSGTAGAALLALLAALCPASR

SEQ ID NO: 119 - protein, 621 aa, synthetic construct [sequence omitted]

SEQ ID NO: 120 - protein, 19 aa, synthetic construct
STWWKEPLNIVSFLFCFAS

SEQ ID NO: 121 - protein, 1408 aa, synthetic construct
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 122 - protein, 24 aa, synthetic construct
MKAPAVLAPGILVLLFTLVQRSNG

SEQ ID NO: 123 - protein, 908 aa, synthetic construct
ECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFT

SEQ ID NO: 124 - protein, 23 aa, synthetic construct
GLIAGVVSISTALLLLLGFFLWL

SEQ ID NO: 125 - protein, 434 aa, synthetic construct
KKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 126 - protein, 16 aa, synthetic construct
EGYYETTTYYYNLFDS

SEQ ID NO: 127 - protein, 10 aa, synthetic construct
DRNWGWDFDY

SEQ ID NO: 128 - protein, 11 aa, synthetic construct
GDLFITGTLDY

SEQ ID NO: 129 - protein, 14 aa, synthetic construct
KLEPTGYYYYYMDV

SEQ ID NO: 130 - protein, 14 aa, synthetic construct
KTSRYSGYHYYMDV

SEQ ID NO: 131 - protein, 8 aa, synthetic construct
AHYDILTG

SEQ ID NO: 132 - protein, 12 aa, synthetic construct
GKSHYSWDAFDY

SEQ ID NO: 133 - protein, 20 aa, synthetic construct
GTYYYGSGSFSTRVFDAFDV

SEQ ID NO: 134 - protein, 14 aa, synthetic construct
QSRRYSGYASYFDY

SEQ ID NO: 135 - protein, 14 aa, synthetic construct
QRRAYSGYNWYFDL

SEQ ID NO: 136 - protein, 12 aa, synthetic construct
RNDFWSGYLFDY

SEQ ID NO: 137 - protein, 13 aa, synthetic construct
KTTVGYYYYYMDV

SEQ ID NO: 138 - protein, 13 aa, synthetic construct
GPELGYYYYYMDI

SEQ ID NO: 139 - protein, 17 aa, synthetic construct
ASSMITFGGVIVSWFDP

SEQ ID NO: 140 - protein, 14 aa, synthetic construct
RVNRYSGYATYFDL

SEQ ID NO: 141 - protein, 14 aa, synthetic construct
ETYYYDRGGYPFDP

SEQ ID NO: 142 - protein, 14 aa, synthetic construct
RTSRYSGYHYYLDV

SEQ ID NO: 143 - protein, 8 aa, synthetic construct
LLYDLFDL

SEQ ID NO: 144 - protein, 13 aa, synthetic construct
SIDMATITDAFDI

SEQ ID NO: 145 - protein, 15 aa, synthetic construct
GTTGNPYYFYYYMDV

(19) United States
(12) Patent Application Publication
(10) Pub. No.: US 2025/0179193 A1
(43) Pub. Date: Jun. 5, 2025

(54) TREATMENT WITH AN ANTIBODY THAT BINDS EGFR AND CMET

(71) Applicant: MERUS N.V., Utrecht (NL)

(72) Inventors: Jeroen Jilles LAMMERTS VAN BUEREN, Utrecht (NL); Gianluca LAUS, Utrecht (NL); Benjamin Awori BARASA, Utrecht (NL)

(21) Appl. No.: 18/844,527

(22) PCT Filed: Mar. 7, 2023
(86) PCT No.: PCT/NL2023/050111

§ 371 Date: Sep. 6, 2024

(30) Foreign Application Priority Data
Mar. 7, 2022 (NL) 2031178
Jul. 6, 2022 (NL) 2032395
Nov. 2, 2022 (NL) 2033440

(51) Int. Cl. C07K 16/28; A61K 39/00; A61P 35/04

(52) U.S. Cl. CPC C07K 16/2863; A61P 35/04; A61K 2039/505; C07K 2317/21; C07K 2317/31; C07K 2317/526; C07K 2317/565; C07K 2317/732; C07K 2317/76

(57) ABSTRACT

The invention as disclosed herein relates to a bispecific antibody that comprises a 5 first variable domain that can bind an extracellular part of human epidermal growth factor receptor (EGFR) and a second variable domain that can bind an extracellular part of human MET Proto-Oncogene, Receptor Tyrosine Kinase (cMET) for use in a method of treatment of a cancer in a subject which has received prior treatment with i) a third-generation EGFR tyrosine kinase inhibitor, or ii) a chemotherapy and a tyrosine kinase 10 inhibitor or iii) a cMET tyrosine kinase inhibitor.

The disclosure relates to the field of antibodies. In particular it relates to the field of therapeutic antibodies, including human antibodies, for the treatment of diseases involving aberrant cells. Further, it relates to antibodies that bind EGFR and cMET, including multispecific antibodies, and their use in the binding of EGFR and cMET positive cells, particularly tumor cells.

The epidermal growth factor (EGF) receptor (EGFR) is a cell-surface receptor for members of the epidermal growth factor family (EGF-family) of extracellular protein ligands. EGFR is also known as the ErbB-1 receptor. The receptor has been given various names in the past (EGFR; ERBB; ERBB1; HER1; PIG61; mENA). In the present disclosure the names ErbB-1, EGFR or HER1 in humans will be used interchangeably. EGFR is a member of the ErbB family of receptors, a subfamily of four closely related receptor tyrosine kinases: ErbB-1 (EGFR), ErbB-2 (HER2/c-neu; Her2), ErbB-3 (Her 3) and ErbB-4 (Her 4).

EGFR exists on a cell surface and may be activated by binding of its specific ligands, including epidermal growth factor and transforming growth factor α (TGFα). Upon activation by its growth factor ligands, the receptor may undergo a transition from an inactive mostly monomeric form to an active homodimer. In addition to forming homodimers after ligand binding, EGFR may pair with another member of the ErbB receptor family, such as ErbB2, to create an activated heterodimer. Dimers may also form in the absence of ligand-binding and clusters of activated EGFRs may form after ligand binding.

EGFR dimerization stimulates intrinsic intracellular protein-tyrosine kinase (PTK) activity. This activity induces several signal transduction cascades that lead to cell proliferation and differentiation. The kinase domain of EGFR can cross-phosphorylate tyrosine residues of other receptors it is complexed with, and can itself be activated in that manner.

Mutations involving EGFR have been identified in several types of cancer. It is the target of an expanding class of anticancer therapies. Such therapies include EGFR tyrosine kinase inhibitors (EGFR-TKIs) such as gefitinib and erlotinib for lung cancer, and antibodies as cetuximab and panitumumab for colon cancer and head and neck cancer.

Cetuximab and panitumumab are monoclonal antibodies that inhibit the receptor. Other monoclonals in clinical development are zalutumumab, nimotuzumab, and matuzumab. The monoclonal antibodies aim to block the extracellular ligand-induced receptor activation, mostly by blocking ligand binding to the receptor. With the binding site blocked, signal-inducing molecules may not attach effectively and thereby also not activate downstream signaling. Ligand-induced receptor activation may also be inhibited by stabilization of the inactive receptor conformation (matuzumab).

To date, EGFR targeted therapies have been associated with the development of treatment resistance over time. Various mechanisms for the resistance to EGFR-TKIs have been described. In patients with advanced non-small cell lung cancer (NSCLC) the mechanisms of resistance include the occurrence of secondary or tertiary mutations (e.g., T790M, C797S, L718Q, exon 20 insertion mutations), the activation of alternative signaling (e.g., Met, HGF, AXL, Hh, IGF-1R), aberrant downstream pathways (e.g., AKT mutations, loss of PTEN), the impairment of the EGFR-TKIs-mediated apoptosis pathway (e.g., BCL2-like 11/BIM deletion polymorphism) and histological transformation. Although some mechanisms of resistance have been identified others remain to be identified. Furthermore, in the case of third-generation TKI resistance mechanisms, the molecular heterogeneity of NSCLC influences the contribution to the wide spectrum of resistance aberrations discovered so far. Similarly, patients with colorectal cancer that are treated with EGFR antibodies also develop resistance over time. This may occur through emergence of KRAS mutations. Of those without KRAS mutations; amplification of the MET proto-oncogene may be associated with acquired resistance during anti-EGFR therapy (Bardelli et al., 2013; Cancer Discov. June; 3(6):658-73. doi: 10.1158/2159-8290.CD-12-0558). The tumor can be resistant ab initio or develop resistance during treatment. Resistance to EGFR-targeted therapy is seen in many EGFR positive cancers and has demonstrated a need in the art for more efficacious EGFR cancer treatments that improve the standard of care, and are superior in terms of the capacity to address EGFR-targeted therapy resistance.

Dysregulation of MET Proto-Oncogene, Receptor Tyrosine Kinase (cMET) and hepatocyte growth factor (HGF) have been reported in a variety of tumors. Ligand-driven cMET activation has been observed in several cancers. Elevated serum and intra-tumoral HGF is observed in lung, breast cancer, and multiple myeloma (J. M. Siegfried et al., Ann Thorac Surg 66, 1915 (1998); P. C. Ma et al., Anticancer Res 23, 49 (2003); B. E. Elliott et al. Can J Physiol Pharmacol 80, 91 (2002); C. Seidel, et al, Med Oncol 15, 145 (1998)). Overexpression of cMET, cMET amplification or mutation has been reported in various cancers such as colorectal, lung, gastric, and kidney cancer and may drive ligand-independent receptor activation (C. Birchmeier et al, Nat Rev Mol Cell Biol 4, 915 (2003); G. Maulik et al., Cytokine Growth Factor Rev 13, 41 (2002)). Expression of HGF is also associated with the activation of the HGF/cMET signaling pathway and is also one of the escape mechanisms of tumors under selection by EGFR targeted therapy. Furthermore, treatment with cMET tyrosine kinase inhibitors, such as capmatinib or tepotinib, has been associated with the emergence of an escape mechanism to cMET aberrations.

The cMET receptor is formed by proteolytic processing of a common precursor into a single-pass, disulphide-linked a/B heterodimer. The extracellular portion of cMET is composed of three domain types. The N-terminal region fold forms a large semaphorin (Sema) domain, which encompasses the whole α-subunit and part of the B-subunit. The plexin-semaphorin-integrin (PSI) domain follows the Sema domain, and includes four disulphide bonds. This domain is connected to the transmembrane helix via four immunoglobulin-plexin-transcription (IPT) domains, which are related to immunoglobulin-like domains. Intracellularly, the cMET receptor contains a tyrosine kinase catalytic domain flanked by distinctive juxtamembrane and carboxy-terminal sequences (Organ and Tsao. Therapeutic advances in medical oncology 3.1_suppl (2011): S7-S19 which is incorporated herein by reference in its entirety).

The ligand of cMET, hepatocyte growth factor (HGF; also known as scatter factor) and its splicing isoforms (NK1, NK2) are known ligands of the cMET receptor. HGF was identified in 1991 as a potent mitogen/morphogen. The HGF/cMET signaling pathway plays important roles in the development and progression of various cancers. Dysregulation and/or hyperactivation of HGF or cMET in human cancers are linked to poor prognosis. cMET can be activated via overexpression, amplification, or mutation. Activation may promote development, progression, invasive growth, and metastasis of cancers. cMET can be activated in an HGF associated and HGF independent fashion. HGF independent activation occurs in cases of cMET over-expression. Abundance of cMET also may trigger (hetero) dimerization and intra-cellular signaling in the absence of ligand. Additional ligand does not appear to affect the function of such cMET over-expression cells. cMET amplification is associated with cMET over-expression and has emerged as a biomarker of tumor subtypes.

HGF is expressed ubiquitously throughout the body, showing this growth factor to be a systemically available cytokine as well as coming from the tumor stroma. A positive paracrine and/or autocrine loop of cMET activation can lead to further cMET expression. The HGF specific antibody Rilotumumab (AMG102) was developed for gastric cancer. Phase I and Phase II trials appeared promising but a phase III study with cisplatin and capecitabine as a first-line therapy in gastric cancer (RILOMET-2) was terminated following a pre-planned data monitoring committee safety review of study 20070622.

The relevance of cMET/HGF signaling in resistance to EGFR-targeted therapies has stimulated the development of ways to deal with the resistance. To date, antibody based approaches include anti-HGF antibodies; anti cMET or cMET antibodies and cMET/EGFR (reviewed in Lee et al., 2015; Immunotargets and Therapy 4:35-44) have not been clinically effective. The cMET antibodies Onartuzumab (MetMab™) and Emibetuzumab (LY-2875358) have been evaluated in phase II clinical trials. Of these Onartuzumab appeared to be effective against colorectal cancer in a combination treatment together with the EGFR-inhibitor erlotinib. These results could, however, not be repeated in a randomized phase III clinical trial. MetMAb is a monovalent monoclonal antibody (mAb) against cMET, which blocks HGF binding to cMET and subsequent pathway activation (Jin et al., 2008 Cancer Research Vol. 68: pp 4360-68).

Overcoming a problem with anti-EGFR, cMET and HGF immunotherapies, the present disclosure provides novel bispecific antibodies that comprise a first variable domain that can bind an extracellular part of epidermal growth factor receptor (EGFR) and a second variable domain that can bind an extracellular part of cMET Proto-Oncogene, Receptor Tyrosine Kinase (cMET).

To date, certain bispecific EGFR×cMET antibodies have been described in the art. Castoldi R. et al. (2013) describe a bispecific EGFR×cMET antibody designated MetHer1 with the cMET binding site of the antibody 5D5 (or MetMab) and the EGFR binding site of cetuximab. The bispecific antibody has a fixed EGFR and cMET binding stoichiometry of 2:1 (see Supplemental Figures)

US20140378664 describes a cMET×EGFR bispecific antibody among various other bispecific antibodies. The complete bispecific antibody is produced as a single protein which is later proteolytically cleaved. The two VH/VL domains are produced as single chain Fv fragments. Binding of the antibody induces cMET degradation and Akt phosphorylation in a gastric cancer cell line. Moores et al (2016) describe a bispecific cMET×EGFR antibody designated JNJ-61186372 produced by controlled Fab-arm exchange (cFAE) having mutations at position 405 and 409 according to EU numbering, which may have potential for immunogenicity. JNJ-61186372 was shown to be active in vivo using a xenograft model with tumor cell line H1975 that expresses the cMET ligand HGF. This tumor model is known to be dependent on the ADCC activity of the antibody (Ahmed et al., 2015). JNJ-61186372 has a reported affinity imbalance of approximately 40× greater affinity for cMET than EGFR (Moores et al. (2016)), and the anti-EGFR arm derived from zalutumumab is known to cause infusion related reaction, skin disorders, among other issues.

LY3164530 is a bispecific cMET×EGFR antibody, which contains the EGFR binding domain of cetuximab as a single chain Fv fragment fused to the heavy chain variable domain of the cMET binding antibody LY2875358 (Emibetuzumab; Kim and Kim 2017). It is a so-called dual variable domain antibody that comprises two binding sites for each of the antigens. No data are provided on HGF inhibition for the antibody. The antibody reportedly binds and internalizes cMET and EGFR without agonistic activity. The authors review various cMET, EGFR and cMET×EGFR targeted therapies and draw the conclusion that to date none of these inhibitors have shown significant efficacy in clinical trials.

There is thus a need for novel bispecific cMET×EGFR antibodies, including those which may have superior characteristics as described herein.

### SUMMARY OF THE INVENTION

In certain aspects, the present disclosure provides a bispecific antibody that comprises a first variable domain that can bind an extracellular part of human epidermal growth factor receptor (EGFR) and a second variable domain that can bind an extracellular part of human MET Proto-Oncogene, Receptor Tyrosine Kinase (cMET) for use in a method of treatment of a cancer in a subject which has received prior treatment with i) a third-generation EGFR tyrosine kinase inhibitor.

In certain aspects, the present disclosure provides a bispecific antibody that comprises a first variable domain that can bind an extracellular part of human epidermal growth factor receptor (EGFR) and a second variable domain that can bind an extracellular part of human MET Proto-Oncogene, Receptor Tyrosine Kinase (cMET) for use in a method of treatment of a cancer in a subject which has received prior treatment with ii) a chemotherapy and a tyrosine kinase inhibitor.

In certain aspects, the present disclosure provides a bispecific antibody that comprises a first variable domain that can bind an extracellular part of human epidermal growth factor receptor (EGFR) and a second variable domain that can bind an extracellular part of human MET Proto-Oncogene, Receptor Tyrosine Kinase (cMET) for use in a method of treatment of a cancer in a subject which has received prior treatment with iii) a cMET tyrosine kinase inhibitor.

In certain aspects, the present disclosure provides a bispecific antibody that comprises a first variable domain that can bind an extracellular part of human epidermal growth factor receptor (EGFR) and a second variable domain that can bind an extracellular part of human MET Proto-Oncogene, Receptor Tyrosine Kinase (cMET) for use in a method of treatment of a cancer in a subject which iv) has not received prior anticancer treatment. In certain aspects, said cancer comprises a cMET exon 14 skipping mutation.

In certain aspects, the present disclosure provides a treating a cancer in a subject which has received prior treatment with i) a third-generation EGFR tyrosine kinase inhibitor, said treatment comprising administering to the subject an effective amount of the bispecific antibody according to the present disclosure that comprises a first variable domain that can bind an extracellular part of human epidermal growth factor receptor (EGFR) and a second variable domain that can bind an extracellular part of human MET Proto-Oncogene, Receptor Tyrosine Kinase (cMET).

In certain aspects, the present disclosure provides a treating a cancer in a subject which has received prior treatment with ii) a chemotherapy and an EGFR tyrosine kinase inhibitor, said treatment comprising administering to the subject an effective amount of the bispecific antibody according to the present disclosure that comprises a first variable domain that can bind an extracellular part of human epidermal growth factor receptor (EGFR) and a second variable domain that can bind an extracellular part of human MET Proto-Oncogene, Receptor Tyrosine Kinase (cMET).

In certain aspects, the present disclosure provides a treating a cancer in a subject which has received prior treatment with iii) a cMET tyrosine kinase inhibitor, said treatment comprising administering to the subject an effective amount of the bispecific antibody according to the present disclosure that comprises a first variable domain that can bind an extracellular part of human epidermal growth factor receptor (EGFR) and a second variable domain that can bind an extracellular part of human MET Proto-Oncogene, Receptor Tyrosine Kinase (cMET).

In certain aspects, the present disclosure provides a treating a cancer in a subject which iv) has not received any prior anticancer treatment, said treatment comprising administering to the subject an effective amount of the bispecific antibody according to the present disclosure that comprises a first variable domain that can bind an extracellular part of human epidermal growth factor receptor (EGFR) and a second variable domain that can bind an extracellular part of human MET Proto-Oncogene, Receptor Tyrosine Kinase (cMET). In certain aspects, said cancer comprises a cMET exon 14 skipping mutation.

In certain aspects, the present disclosure provides the use of the bispecific antibody according to the present disclosure that comprises a first variable domain that can bind an extracellular part of human epidermal growth factor receptor (EGFR) and a second variable domain that can bind (or binds) an extracellular part of human MET Proto-Oncogene, Receptor Tyrosine Kinase (cMET) in the manufacture of a medicament for the treatment of a cancer in a subject which has received prior treatment with i) a third-generation EGFR tyrosine kinase inhibitor.

In certain aspects, the present disclosure provides the use of the bispecific antibody according to the present disclosure that comprises a first variable domain that can bind an extracellular part of human epidermal growth factor receptor (EGFR) and a second variable domain that can bind (or binds) an extracellular part of human MET Proto-Oncogene, Receptor Tyrosine Kinase (cMET) in the manufacture of a medicament for the treatment of a cancer in a subject which has received prior treatment with ii) a chemotherapy and an EGFR tyrosine kinase inhibitor.

In certain aspects, the present disclosure provides the use of the bispecific antibody according to the present disclosure that comprises a first variable domain that can bind an extracellular part of human epidermal growth factor receptor (EGFR) and a second variable domain that can bind (or binds) an extracellular part of human MET Proto-Oncogene, Receptor Tyrosine Kinase (cMET) in the manufacture of a medicament for the treatment of a cancer in a subject which has received prior treatment with iii) a cMET tyrosine kinase inhibitor.

In certain aspects, the present disclosure provides the use of the bispecific antibody according to the present disclosure that comprises a first variable domain that can bind an extracellular part of human epidermal growth factor receptor (EGFR) and a second variable domain that can bind (or binds) an extracellular part of human MET Proto-Oncogene, Receptor Tyrosine Kinase (cMET) in the manufacture of a medicament for the treatment of a cancer in a subject which iv) has not received any prior anticancer treatment. In certain aspects, said cancer comprises a cMET exon 14 skipping mutation.

In certain aspects, the cancer is an EGFR positive cancer, a cMET positive cancer or an EGFR and cMET positive cancer. In certain aspects, the cancer comprises an EGFR aberration, a cMET aberration or an EGFR and cMET aberration.

In certain aspects, the subject or cancer which has received prior treatment comprising a third-generation EGFR tyrosine kinase inhibitor according to i) is resistant to treatment with a third-generation EGFR tyrosine kinase inhibitor.

In certain aspects, the subject or cancer which has received prior treatment comprising chemotherapy and an EGFR tyrosine kinase inhibitor according to ii) is resistant to treatment with a first-, second and/or third-generation tyrosine kinase inhibitor.

In certain aspects, the subject or cancer which has received prior treatment comprising a cMET tyrosine kinase inhibitor according to iii) is resistant to treatment with a cMET tyrosine kinase inhibitor.

In certain aspects, the administration of said bispecific antibody according to i) is administered as second line treatment, following treatment with a third generation EGFR tyrosine kinase inhibitor.

In certain aspects, the administration of said bispecific antibody according to ii) is administered as third line treatment following chemotherapy and treatment with an EGFR tyrosine kinase inhibitor.

In certain aspects, the administration of said bispecific antibody according to iii) is administered as second line treatment following treatment with a cMET tyrosine kinase inhibitor.

In certain aspects, said cancer or subject has received prior treatment with a third-generation EGFR tyrosine kinase inhibitor and/or said cancer or subject is resistant to treatment with a third-generation EGFR tyrosine kinase inhibitor. Preferably, said subject comprises an EGFR aberration, a cMET aberration or an EGFR and cMET aberration conferring resistance to said third-generation EGFR tyrosine kinase inhibitor. In certain aspects, said third-generation EGFR tyrosine kinase inhibitor comprises or is Osimertinib.

In certain aspects, said cancer or subject has received prior treatment with a chemotherapy and a first or second or third generation tyrosine kinase inhibitor. In certain aspects, said cancer or subject is resistant to treatment with a first or second or third generation tyrosine kinase inhibitor, or resistant to a first and second generation tyrosine kinase inhibitor or resistant to a first, second and third generation tyrosine kinase inhibitor. In certain aspects, said subject comprises an EGFR aberration, or a cMET aberration or an EGFR and cMET aberration conferring resistance to said first, -second- or third-generation EGFR tyrosine kinase inhibitor.

In certain aspects, said cancer or subject has received prior treatment with a cMET tyrosine kinase inhibitor, and/or said cancer or subject is resistant to treatment with said cMET tyrosine kinase inhibitor. In certain aspects, said subject comprises a cMET aberration conferring resistance to said cMET tyrosine kinase inhibitor. In certain aspects, said cMET inhibitor comprises capmatinib or tepotinib.

In certain aspects, said subject comprises an EGFR and/or cMET aberration conferring resistance to said third-generation EGFR tyrosine kinase inhibitor. In certain aspects, said third-generation EGFR tyrosine kinase inhibitor comprises or is Osimertinib.

In in certain aspects, the third-generation EGFR tyrosine kinase inhibitor which is provided as prior treatment comprises or is Osimertinib, Lazertinib, Alflutinib, Rezivertinib, Rociletinib, Olmutinib, Almonertinib, Abivertinib, ASK120067, Befotertinib (also referred to as BPI-D0316 or D-0316), SH-1028, Nazartinib (EGF816), Naquotinib (ASP8273), Mavelertinib (PF-0647775), Olafertinib (CK-101), Keynatinib, ES-072, in certain aspects Osimertinib. In certain aspects, said EGFR tyrosine kinase inhibitor is Osimertinib, BPI-D0316/Befotertinib, Lazertinib or Almonertinib.

In certain aspects, said first-generation EGFR tyrosine kinase inhibitor which is provided as prior treatment with said chemotherapy comprises or is gefitinib, erlotinib, or icotinib.

In certain aspects, said second-generation tyrosine kinase inhibitor which is provided as prior treatment comprises or is afatinib, dacomitinib, XL647, AP26113, CO-1686 or neratinib.

In certain aspects, said cMET tyrosine kinase inhibitor which is provided as prior treatment is or comprises capmatinib, tepotinib, crizotenib, cabozantinib, savolitinib, Glesatinib, Sitravatinib, BMS-777607, Merestinib, Tivantinib, Golvatinib, Foretinib, AMG-337 or BMS-794833.

In certain aspects, said prior received chemotherapy comprises platinum-based chemotherapy, cisplatin, carboplatin, oxaliplatin, paclitaxel, docetaxel, gemcitabine, vinorelbine, etoposide or pemetrexed, or any combination thereof, in certain aspects a combination comprising cisplatin or carboplatin.

In certain aspects, the subject is a human subject.

In certain aspects, said cancer is non-small cell lung cancer (NSCLC), head and neck cancer, in particular head and neck squamous cell carcinoma; gastric cancer, in particular gastric adenocarcinoma; or esophageal cancer, in particular esophageal squamous cell carcinoma; gastric/esophageal junction cancer, breast cancer, colon cancer, pancreatic cancer, ovarian cancer, colorectal cancer or bladder cancer. In certain aspects, said cancer or subject comprises an activating EGFR mutation, an approved tyrosine kinase inhibitor resistance mutation, a tertiary tyrosine kinase inhibitor resistance mutation, a mutation that reduces binding of a third generation tyrosine kinase inhibitor to EGFR, an acquired tyrosine kinase inhibitor resistance mutation, an EGFR gene amplification, a cMET mutation, cMET aberration or increased HGF expression.

In certain aspects, said cancer or subject comprises an in-frame EGFR exon 20 insertion mutation.

In certain aspects, cancer comprises a cMET aberration, such as a cMET amplification, cMET overexpression, increased signaling of the cMET pathway, a cMET gene amplification and/or increased cMET protein activity. In certain aspects, said cancer comprises increased HGF expression. In certain aspects, said cancer comprises a cMET exon14 skipping mutation.

In certain aspects, the cMET dysregulation comprises a cMET amplification, cMET overexpression, increased signaling of the cMET pathway, a cMET gene amplification, or increased cMET protein activity or cMET dysregulation caused by increased HGF expression. In certain aspects, said cMET aberration comprises a cMET exon14 skipping mutation. cMET dysregulation in certain aspects is or comprises dysregulation of cMET signaling.

In certain aspects, the use or treatment comprises providing the subject with a dose of 1000, 1500 or 2000 mg of the bispecific antibody. In certain aspects, said bispecific antibody is provided once every week or once every two weeks. In certain aspects, the use or treatment comprises providing the subject with a dose of 1500 mg of the bispecific antibody once every two weeks.

In certain aspects, a bispecific antibody of the disclosure exhibits ADCC activity, in certain aspects the antibody has improved ADCC activity. In such aspect the antibody can have altered ADCC activity by means of one or more CH2 mutations. Further provided is therefore a bispecific antibody according to the disclosure, which is afucosylated. In certain aspects, the antibody of the present disclosure comprises two afucosylated CH2 domains. In certain aspects, the antibody of the present disclosure comprises a total of two CH2 domains, both of which are afucosylated. In certain aspects, the antibody of the present disclosure comprises two CH2 domains, both of which are afucosylated.

In certain aspects, a bispecific antibody of the disclosure exhibits ADCP activity, in certain aspects the antibody has improved ADCP activity. In certain aspects, both the EGFR and cMET binding arms, or both heavy chains that comprise the EGFR and cMET binding arms, contribute to ADCP. In certain aspects, the bispecific antibody of the present disclosure has or exhibits ADCP activity towards NSCLC cells. In certain aspects, the bispecific antibody of the present disclosure induces ADCP of NSCLC cells.

The bispecific antibody may comprise a common light chain. The first and second variable domains comprise the same or substantially the same (common) light chain variable region in certain aspects. Said common light chain variable region may be one that is known to pair well with a diversity of human variable region gene segments that have undergone recombination. In certain aspects, said common light chain is a variable region encoded by a germline Vk gene segment, such as the O12/IgVκ1-39*01 variable region gene segment. The preferred light chain variable region comprises the rearranged IgVκ1-39*01/IGJK1*01 or IgVκ1-39*01/IGJK5*01. The light chain of the cMET binding arm and the light chain of the EGFR binding arm is the same (common) light chain in certain aspects. In certain aspects, the common light chain is the rearranged kappa light chain IgVκ1-39*01/IGJK1*01 or IgVκ1-39*01/IGJK5*01 joined to a human light chain constant region. The bispecific antibody can be a human antibody. The bispecific antibody can be a full length antibody. It may have one variable domain that can bind EGFR and one variable domain that can bind cMET. In certain aspects the variable domain that can bind human EGFR can also beneficially bind mouse EGFR and/or cynomolgus EGFR. In certain aspects, the variable domain that binds or can bind human EGFR binds to domain III of human EGFR. The variable domain that can bind cMET may block the binding of antibody 5D5 to cMET. The variable domain that can bind cMET may block the binding of HGF to cMET. The Kd of the antibody for cMET can be at least 10 times less than the Kd of the antibody for EGFR. The amino acids at positions 405 and 409 in one CH3 domain may be the same as the amino acids at the corresponding positions in the other CH3 domain (EU-numbering).

In certain aspects, the antibody of the present disclosure comprises a first variable domain that comprises a heavy chain variable region with a CDR1 sequence SYGIS; a CDR2 sequence WISAYX₁X₂NTNYAQKLQG and a CDR3 comprising the sequence X₃X₄X₅X₆HWWLX₇A
wherein X₁=N or S; X₂=A or G; X₃=D or G; X₄=R, S or Y; X₅=H, L or Y; X₆=D or W and X₇=D or G; with 0-5 amino acid insertions, deletions, substitutions, additions or a combination thereof at a position other than X₁-X₇.

In certain aspects, the antibody of the present disclosure comprises a second variable domain which comprises a heavy chain variable region with the amino acid sequence of one of the sequences of SEQ ID NO: 1-23 with 0-10, or 0-5 amino acid insertions, deletions, substitutions, additions or a combination thereof.

Bispecific antibodies are described wherein
X₁=N; X₂=G; X₃=D; X₄═S; X₅=Y; X₆=W and X₇=G;
X₁=N; X₂=A; X₃=D; X₄═S; X₅=Y; X₆=W and X₇=G;
X₁═S; X₂=G; X₃=D; X₄═S; X₅=Y; X₆=W and X₇=G;
X₁=N; X₂=G; X₃=D; X₄=R; X₅=H; X₆=W and X₇=D;
X₁=N; X₂=A; X₃=D; X₄=R; X₅=H; X₆=W and X₇=D;
X₁═S; X₂=G; X₃=D; X₄=R; X₅=H; X₆=W and X₇=D;
X₁=N; X₂=G; X₃=G; X₄=Y; X₅=L; X₆=D and X₇=G;
X₁=N; X₂=A; X₃=G; X₄=Y; X₅=L; X₆=D and X₇=G; or
X₁═S; X₂=G; X₃=G; X₄=Y; X₅=L; X₆=D and X₇=G.

In certain aspects, X₁=N; X₂=G; X₃=D; X₄=R; X₅=H; X₆=W and X₇=D; or X₁=N; X₂=A; X₃=D; X₄=R; X₅=H; X₆=W and X₇=D; or X₁═S; X₂=G; X₃=D; X₄=R; X₅=H; X₆=W and X₇=D.

In certain aspects X₃-X₇=DRHWD and X₁ and X₂ are NG; SG or NA.

Bispecific antibodies are described wherein the heavy chain variable region of the second variable domain comprises the amino acid sequence of one of the sequences of SEQ ID NO: 1-3; 7; 8; 10; 13; 15; 16; 17; 21; 22 or 23 with 0-10, or 0-5 amino acid insertions, deletions, substitutions, additions or a combination thereof. One advantage of the present disclosure is that in certain aspects, bispecific antibodies of the present disclosure show more potent ADCC activity than amivantamab, especially for cells or a cancer comprising a cMET aberration.

The disclosure also provides a method of treatment of a subject that has a tumor the method comprising administering the bispecific antibody as described herein to the individual in need thereof. Typically, the individual is one suffering from a disease involving aberrant cells, for examples the individual may be suffering from a tumor or a cancer.

The disclosure also provides a bispecific antibody that comprises a first variable domain that can bind an extracellular part of epidermal growth factor receptor (EGFR) and a second variable domain that can bind an extracellular part of MET Proto-Oncogene, Receptor Tyrosine Kinase (cMET), wherein the first variable domain comprises a heavy chain variable region with a CDR1 sequence SYGIS; a CDR2 sequence WISAYX₁X₂NTNYAQKLQG and a CDR3 comprising the sequence X₃X₄X₅X₆HWWLX₇A wherein
X₁=N or S; X₂=A or G; X₃=D or G; X₄=R, S or Y; X₅=H, L or Y; X₆=D or W and X₇=D or G with 0-5 amino acid insertions, deletions, substitutions, additions or a combination thereof at a position other than X₁-X₇ and wherein the second variable domain comprises a heavy chain variable region with the amino acid sequence of one of the sequences of SEQ ID NO: 1-23 with 0-10, or 0-5 amino acid insertions, deletions, substitutions, additions or a combination thereof.

In certain aspects, the first variable domain comprises a heavy chain variable region with a CDR1 sequence SYGIS; a CDR2 sequence WISAYNGNTNYAQKLQG and a CDR3 comprising the sequence DRHWHWWLDAFDY and the second variable domain in certain aspects comprises a heavy chain variable region with a CDR1 sequence SYSMN; a CDR2 sequence WINTYTGDPTYAQGFTG and a CDR3 sequence ETYYYDRGGYPFDP.

The disclosure also provides a bispecific antibody for use in the treatment of a subject that has a disease involving aberrant cells, such as a tumor.

Also provided is a use of a bispecific antibody of the present disclosure in the manufacture of a medicament for the treatment of a disease involving aberrant cells, such as a tumor or cancer in a subject which has received prior treatment with i) a third-generation EGFR tyrosine kinase inhibitor or ii) a chemotherapy and an EGFR tyrosine kinase inhibitor or iii) a cMET tyrosine kinase inhibitor.

An antibody of the present disclosure inhibits HGF and EGF/HGF induced growth of a cancer which has received prior treatment with i) a third-generation EGFR tyrosine kinase inhibitor or ii) a chemotherapy and an EGFR tyrosine kinase inhibitor or iii) a cMET tyrosine kinase inhibitor

In certain aspects, an antibody of the present disclosure inhibits HGF induced growth of an HGF responsive cell, in certain aspects of an EGFR TKI resistant or refractory tumor of a human subject, tumor model or cell line, such as a cell line or model including an activating EGFR mutation, an approved tyrosine kinase inhibitor resistance mutation, a tertiary tyrosine kinase inhibitor resistance mutation, a mutation that reduces binding of a third generation tyrosine kinase inhibitor to EGFR, an acquired tyrosine kinase inhibitor resistance mutation, an EGFR gene amplification, a cMET mutation or cMET aberration, in certain aspects an in-frame exon 20 insertion mutation. In certain aspects, inhibition is shown in the presence of HGF.

In certain aspects, an antibody of the present disclosure inhibits HGF induced growth of an HGF responsive cell, in certain aspects of a tumor of a human subject, a tumor model or cell line comprising an activating EGFR mutation, an approved tyrosine kinase inhibitor resistance mutation, a tertiary tyrosine kinase inhibitor resistance mutation, a mutation that reduces binding of a third generation tyrosine kinase inhibitor to EGFR, an acquired tyrosine kinase inhibitor resistance mutation, an EGFR gene amplification, a cMET mutation, cMET aberration or an in-frame exon 20 insertion mutation. In certain aspects, inhibition is shown in the presence of HGF.

An antibody of the present disclosure inhibits EGF induced growth of an EGF responsive cell, without inducing the toxicities such as rash and diarrhea associated with high affinity bivalent EGFR antibodies. This renders the antibody ideally suited for combination with TKI which have its own toxicity profile.

An antibody of the present disclosure may be used to treat a tumor which is resistant to treatment with an EGFR tyrosine kinase inhibitor, for example resistant or refractory to Osimertinib, erlotinib, gefitinib, or afatinib, an analogue of Osimertinib, erlotinib, gefitinib or afatinib or a combination of one or more of the respective compounds and/or analogues thereof.

The disclosure further comprises a nucleic acid molecule or a group of nucleic acid molecules that alone or together encode a heavy chain(s) or a heavy chain variable region(s) of a bispecific antibody disclosed herein or a variant thereof. Also provided is a nucleic acid molecule or group of nucleic acid molecules that encode an antibody disclosed herein.

In certain aspects the heavy chain comprises a constant region of an IgG1 antibody, in certain aspects a human IgG1 antibody. The CH2 region of said IgG1 constant region can be engineered to alter ADCC and/or CDC activity of the antibody, or not. In certain aspects, said alteration results in enhanced ADCC and/or CDC activity. In certain aspects the CH3-region of the antibody is engineered to facilitate heterodimerization of heavy chains comprising a first heavy chain that binds EGFR and a second heavy chain binds cMET.

The disclosure further comprises is a cell comprising one or more nucleic acid molecules that alone or together encode a bispecific antibody or a variant thereof as disclosed herein. Also provided are methods of producing a bispecific antibody or a variant thereof disclosed herein using a cell as described, in certain aspects together with the harvesting of the bispecific antibody or variant thereof from a culture of the cells.

The disclosure further comprises a cell system that comprises a bispecific antibody or variant thereof disclosed herein.

The disclosure further provides a cell that expresses the bispecific antibody and/or comprises the nucleic acid molecule(s) that encode said bispecific antibody.

The disclosure further comprises a bispecific antibody as disclosed herein that further comprises a label, in certain aspects a label for in vivo imaging.

### DETAILED DESCRIPTION OF THE INVENTION

EGFR is a member of a family of four receptor tyrosine kinases (RTKs), named Her- or cErbB-1, -2, -3 and -4. The EGFR has an extracellular domain (ECD) that is composed of four sub-domains, two of which are involved in ligand binding and one of which is involved in homo-dimerization and hetero-dimerization Ferguson (2008). The reference numbers used in this section refer to the numbering of the references in the list headed “cited in the specification”, which are each incorporated by reference. EGFR integrates extracellular signals from a variety of ligands to yield diverse intracellular responses (Yarden at al. 2001; and Jorrisen et al. 2003). The EGFR is implicated in several human epithelial malignancies, notably cancers of the breast, bladder, non-small cell lung cancer lung, colon, ovarian head and neck and brain. Activating mutations in the gene have been found, as well as over-expression of the receptor and of its ligands, giving rise to autocrine activation loops (for review, see Robertson et al. 2000). This RTK has therefore been extensively used as target for cancer therapy. Both small-molecule inhibitors targeting the RTK and monoclonal antibodies (mAbs) directed to the extracellular ligand-binding domains have been developed and have shown hitherto several clinical successes, albeit mostly for a select group of patients. Database accession numbers for the human EGFR protein and the gene encoding it are (GenBank NM_005228.3). Other database identifiers for the gene and/or protein are HGNC: 3236; Entrez Gene: 1956; Ensembl: ENSG00000146648; OMIM: 131550 and UniProtKB: P00533. The accession numbers are primarily given to provide a further method of identification of EGFR protein as a target, the actual sequence of the EGFR protein bound by an antibody may vary, for instance because of a mutation in the encoding gene such as those occurring in some cancers or the like. Where reference herein is made to EGFR, the reference refers to human EGFR unless otherwise stated. The antigen-binding site that binds EGFR, binds EGFR and a variety of variants thereof such as those expressed on some EGFR positive tumors.

The term “EGFR ligand” as used herein refers to polypeptides which bind and activate EGFR. Examples of EGFR ligands include, but are not limited to EGF, TGF-α, HB-EGF, amphiregulin, betacellulin and epiregulin (for review Olayioye M A et al.; EMBO J (2000) Vol 19: pp 3159-3167). The term includes biologically active fragments and/or variants of a naturally occurring polypeptide

Aberrantly activated forms of EGFR, for instance via mutations in EGFR or EGFR gene amplification, are known to be oncogenic drivers in non-small cell lung cancer (NSCLC) and known to occur in the treatment with EGFR tyrosine kinase inhibitors. The present disclosure provides a bispecific antibody for use in a method of treatment of a cancer in a subject which has received prior treatment with i) a third-generation EGFR tyrosine kinase inhibitor or ii) a chemotherapy and a tyrosine kinase inhibitor, iii) a cMET tyrosine kinase inhibitor or iv) a subject which has not received prior anticancer treatment, which subject under iv) comprises in certain aspects a cMET exon 14 skipping mutation or a cancer which comprises a cMET exon 14 skipping mutation.

In certain aspects, treatment comprises treatment of a cancer which results from ligand-independent activation of EGFR and/or ligand-independent activation of cMET. In another aspect, the treatment comprises treatment of a cancer which results from ligand-dependent activation of EGFR and/or ligand-dependent activation of cMET.

In certain aspects, the cancer or subject has received prior treatment with Osimertinib and has an acquired or tertiary Osimertinib resistance. Said prior Osimertinib treatment is in certain aspects first line or second line therapy. In certain aspects, first line therapy is followed by treatment with the bispecific antibody of the present disclosure as second treatment.

In certain aspects, the cancer comprises an activating EGFR mutation, an approved tyrosine kinase inhibitor resistance mutation, a tertiary tyrosine kinase inhibitor resistance mutation, a mutation that reduces binding of a third generation tyrosine kinase inhibitor to EGFR, an acquired tyrosine kinase inhibitor resistance mutation, an EGFR gene amplification, a cMET mutation, cMET aberration or increased HGF expression.

In a clinical study into efficacy of a bispecific antibody of the present disclosure, clinical efficacy was observed in a variety of cancers with different genetic, oncogenic backgrounds. In particular, clinical efficacy was observed in lung cancer, in particular non-small cell lung cancer (NSCLC). For instance, clinical efficacy was observed in patients with various EGFR mutations, including an EGFR exon 20 mutation/insertion, an EGFR exon 21 mutation, such as L858R, an EGFR exon 19 deletion mutation, or a c-MET exon 14 skipping mutation. Thus, in a certain aspect, the cancer is NSCLC and/or the subject suffers from NSCLC which cancer or subject comprises an EGFR exon 21 mutation, such as L858R, an EGFR exon 19 deletion mutation, an EGFR exon 18 mutation or a c-MET exon 14 skipping mutation.

Also, clinical efficacy was observed in head and neck cancer, in particular in head and neck squamous cell carcinoma (HNSCC). Thus, in a certain aspect, the cancer is and/or the subject suffers from head and neck cancer, in particular in head and neck squamous cell carcinoma.

In certain aspects, the cancer is a gastric adenocarcinoma with a c-MET amplification.

In certain aspects, treatment results in or comprises depletion of soluble EGFR and/or cMET.

In certain aspects, the cancer comprises an activating EGFR mutation, such as an in-frame exon 19 deletion mutation or exon 21 mutation (in certain aspects L858R). Herein, the term “activating EGFR mutation” means a mutation which develops after progression on a third-generation EGFR tyrosine kinase inhibitor, including an in-frame deletion in exon 19 (del19) and substitution of leucine for arginine in exon 21 (L858R).

In certain aspects, the cancer comprises an approved tyrosine kinase inhibitor resistance mutation. Herein, the term “approved tyrosine kinase inhibitor resistance mutation” means a resistance which develops after progression on an EGFR tyrosine kinase inhibitor which is presently approved for treatment of cancer, such as T790M conferring resistance to afatinib. In certain aspects, said EGFR tyrosine kinase inhibitor is an approved EGFR tyrosine kinase inhibitor.

In certain aspects, the cancer comprises a tertiary tyrosine kinase inhibitor resistance mutation, such as L718X (e.g. L718Q), G719X (e.g. G719A), L792X (e.g. L792H), G796X (e.g. G796R, G796S, G796D), C797X, C797X (e.g. C797S, C797G). Herein, the term “tertiary tyrosine kinase inhibitor resistance mutation” means a resistance which develops after progression on a third-generation EGFR tyrosine kinase inhibitor, in certain aspects Osimertinib.

In certain aspects, the cancer comprises a mutation that reduces binding of a third generation tyrosine kinase inhibitor to EGFR, such as L792X, L718X.

In certain aspects, the cancer comprises an acquired tyrosine kinase inhibitor resistance mutation, (such as T790M, L858R, an exon 19 deletion mutation, C797X, L792X, G796X, G724X, S768X, L718X or an exon 20 insertion mutation), in certain aspects a mutation which confers resistance to Osimertinib or which occurred after progression on Osimertinib, including G724X (e.g. G724S), S768X (e.g. S768I), L792X (e.g. L792H), C797X (including C797S and C797G), L798X (e.g. L798I). Herein, the term “acquired tyrosine kinase inhibitor resistance mutation” means a resistance which is acquired after progression on treatment with a tyrosine kinase inhibitor, such as after progression on a third-generation EGFR tyrosine kinase inhibitor.

In certain aspects, the cancer comprises an EGFR gene amplification, such as an increase in EGFR mRNA or amplification of the wildtype EGFR allele in combination with the presence of an EGFR-ex19del allele after progression on Osimertinib.

In certain aspects, the cancer comprises a cMET mutation, such as a cMET exon 14 skipping mutation.

In certain aspects, the cancer comprises a cMET aberration, such as a cMET amplification, cMET overexpression, increased signaling of the cMET pathway, a cMET gene amplification, increased HGF expression and/or increased cMET protein activity. In certain aspects, said cancer is NSCLC and said cMET amplification is characterized by MET/CEP7>5 or cfDNA≥2 copies or any combination thereof.

In certain aspects, the cMET amplification is characterized by MET/CEP7≥3, MET/CEP7≥4, or MET/CEP7≥5 (and up to 15 or 20 or less) or by cfDNA≥1.8 cMET copies, such as (≥1.8, <2.2), or (>2.2, <5), or (≥5).

In certain aspects, the cancer comprises an exon 19 deletion mutation, in certain aspects an in-frame exon 19 deletion, an exon 20 missense mutation (e.g. T790M) or an exon 21 mutation, such as L858R.

In certain aspects, the cancer comprises an EGFR exon 20 mutation, in certain aspects an exon 20 insertion mutation, in certain aspects an in-frame exon 20 insertion mutation.

In certain aspects, the cancer or subject comprises an exon 20 mutation selected from a near-loop insertion (positions 767-772), a far-loop insertion (positions 773-775), in certain aspects V769_D770insASV, D770_N771insSVD, H773_V774insNPH, H773_V774insH, D770_N771insG, D770delinsGY, N771_P772insN, V774_C775insHV, D770_N771insGL, H773_V774insPH, A763_Y764insFQEA, D770_N771 delinsEGN, D770_N771insGD, D770_N771insH, D770_N771insP, H773_V774insAH, H773_V774insGNPH, H773delinsSNPY, N771_P772insH, N771_P772insVDN, N771delinsGY, N771delinsKH, N771delinsRD, P772_H773delinsHNPY, P772_H773insGT, P772_H773insPNP, P772_H773insT, V769_D770insA, V769_D770insGG, V769_D770insGSV, V769_D770insGVV and V769_D770insMASV; or mutations T790M, L792X (e.g. L792H), C796X (e.g. G796R, G796S, G796D), C797X (e.g. C797S, C797G), L798I, or an in-frame exon 20 insertion, such as M766_A767insASV or H773-V774insNPH, Ins761 (EAFQ), Ins770 (ASV), Ins771 (G), Ins774 (NPH), M766_A7671 ns A, S768_V769InsSVA, P772_H773InsNS, D761_E762InsX1-7, A763_Y764InsX1-7, Y764_Y765 InsX1-7, M766_A767InsX1-7, A767_V768 InsX1-7, S768_V769 InsX1-7) V769_D770 InsX1-7) D770_N771 InsX1-7) N771_P772 InsX1-7) P772_H773 InsX1-7, H773_V774 InsX1-7, or V774_C775 InsX1-7. In certain aspects, the cancer or subject comprises two or more of said mutations.

In certain aspects, the cancer or subject comprises a mutation such as L718X (e.g. L718Q, L718V), G719X (e.g. G719A), L792X (e.g. L792H, L792F, L792R, L792Y, L792V and L792P), G796X (e.g. G796R, G796S, G796D), C797X, C797X (e.g. C797S, C797G, C797N), C797X, L792X, G796X, G724X, S768X, L718X, M766X (e.g. M766Q), R776X (e.g. R776C) or an exon 20 insertion mutation. In certain aspects, the cancer or subject comprises two or more of said mutations.

In certain aspects, the cancer or subject comprises an exon 19 deletion mutation, in certain aspects an in-frame exon 19 deletion, an exon 20 missense mutation (e.g. T790M) or an exon 21 mutation, such as L858R, L861X (e.g. L861Q) or L844X (e.g. L844V). In certain aspects, the cancer or subject comprises two or more of said mutations.

In certain aspects, the cancer or subject comprises cancer comprises an EGFR exon 20 mutation, in certain aspects an exon 20 insertion mutation.

In certain aspects, the cancer or subject comprises a mutation selected from G724X (e.g. G724S), S768X (e.g. S768I), T790X (e.g. T790M), L792X (e.g. L792H), C797X (including C797S and C797G), L798X (e.g. L798I), 1941X (e.g. 1941R), V948X (e.g. V948R). In certain aspects, the cancer or subject comprises two or more of said mutations.

In certain aspects, the cancer or subject comprises double mutation L858X/T790X (e.g. L858R/T790M), T790X/L798X (e.g. T790M/L798I), T790X/C797X (e.g.

T790M/C797S), G719X/R776X (e.g. G719A/R776C) or delE746_A750/T790M.

In certain aspects, the cancer or subject comprises double mutation D770insSVD/E762X (e.g. E762K), D770insSVD/L792X (e.g. L792I, L792S), D770insSVD/P794X (e.g. P794S), or D770insSVD/G796X (e.g. G796D).

In certain aspects, the cancer or subject comprises double mutation H773insH/E762X (e.g. E762K), H773insH/L792X (e.g. L792I, L792S), H773insH/P794X (e.g. P794S), or H773insH/G796X (e.g. G796D).

In certain aspects, the cancer or subject comprises double mutation H773insNPH/E762X (e.g. E762K), H773insNPH/L792X (e.g. L792I, L792S), H773insNPH/P794X (e.g. P794S), or H773insNPH/G796X (e.g. G796D).

In certain aspects, the cancer or subject comprises double mutation L858X/L718X (e.g. L858R/cis-L718Q), L858X/C797X (e.g. L858R/cis-C797S), exon 19del/C797X (e.g. exon 19del/cis-C797S).

In certain aspects, the cancer or subject comprises triple mutation L858X/T790X/C797X (e.g. L858R/T790M/C797S), L858X/T790X/M766X (e.g. L858R/T790M/M766Q), L858X/T790X/L718X (e.g. L858R/T790M/cis-L718Q, L858R/T790M/L718Q), L858X/T790X/C797X (e.g. L858R/T790M/cis-C797S), exon 19del/T790X/C797X (e.g. exon19del/T790M/cis-C797S), L858X/T790X/C941X (e.g. L858R/T790M/1941R), delE746_A750/T790X/C797X (e.g. delE746_A750/T790M/C797S)

In certain aspects, the cancer or subject comprises cMET exon14 skipping mutation.

Herein, the term “refractory” refers to a disease that does not respond to a treatment. A refractory disease can be resistant to a treatment before or at the beginning of the treatment, or a refractory disease can become resistant during a treatment.

Herein, the term “1st generation EGFR tyrosine kinase inhibitor” (1st generation TKI) refers to reversible EGFR inhibitors such as gefitinib and erlotinib, which are effective in first-line treatment of NSCLC harboring EGFR activating mutations such as deletions in exon 19 and exon 21 L858R mutation.

Herein, the term “2nd generation EGFR tyrosine kinase inhibitor” (2nd generation TKI) refers to covalent irreversible EGFR inhibitors such as afatinib and dacomitib which are effective in first-line treatment of NSCLC harboring EGFR activating mutations such as deletions in exon 19 and exon 21 L858R mutation.

Herein, the term “3rd generation EGFR tyrosine kinase inhibitor” (3rd generation TKI) refers to covalent irreversible EGFR inhibitors such as osimertinib and lazertinib which are selective to the EGFR activating mutations, such as deletions in exon 19 and exon 21 L858R, alone or in combination with T790M mutation and have lower inhibitory activity against wild-type EGFR.

Herein the term “resistant” refers to a cancer or patient which is not responding to treatment when administered to prescribed dose of the therapeutic agent involved, including a 3rd generation EGFR tyrosine kinas inhibitor.

In certain aspects, the third generation EGFR tyrosine kinase inhibitor is Osimertinib (AZD9291). Osimertinib (AZD9291) is a covalent, orally active, irreversible, and mutant-selective EGFR inhibitor with an apparent IC50 of 12 nM against L858R and 1 nM against L858R/T790M, respectively. Recommended phase 2 dosing was established at a daily dose of 80 mg.

In certain aspects, the third generation EGFR tyrosine kinase inhibitor is Almonertinib (HS-10296). Almonertinib is an orally available, irreversible, third-generation EGFR tyrosine kinase inhibitor with selectivity for EGFR-sensitizing and T790M resistance mutations. Almonertinib is used for the research of non-small cell lung cancer. Recommended phase 2 dosing was established at a daily dose of 110 mg.

In certain aspects, the third generation EGFR tyrosine kinase inhibitor is Lazertinib. Lazertinib (YH25448) is a potent, mutant-selective, blood-brain barrier permeable, orally available and irreversible third-generation EGFR tyrosine kinase inhibitor, and can be used in the research of non-small cell lung cancer. Recommended phase 2 dosing was established at a daily dose of 240 mg.

In certain aspects, the third generation EGFR tyrosine kinase inhibitor is Befotertinib (or BPI-D0316 or sometimes D-0316). Befotertinib (Beta Pharmaceuticals, Co., China) is a third-generation EGFR tyrosine kinase inhibitor. Befotertinib can be used for EGFR positive non-small cell lung cancer (NSCLC). In the phase II, single-arm study NCT05007938, the safety and efficacy of befotertinib was assessed at 25 mg three times daily, orally, in combination with icotinib (125 mg three times daily, orally) in patients with locally advanced or metastatic NSCLC. In the phase I study NCT04464551, subjects received a single oral dose of 75 mg D-0316 as an oral suspension. In the phase II, single-arm study NCT03861156, patients with locally advanced/metastatic non-small cell lung cancer received an oral dose of 75 mg for a cycle of 21 days, and if tolerated, dose was increased to 100 mg. Otherwise, the dose was maintained at 75 mg. In the phase II/III study NCT04206072, the efficacy and safety of D-0316 at 70 mg once daily for 21 days, then increased to 100 mg once daily was assessed.

In certain aspects, the third generation EGFR tyrosine kinase inhibitor is Alflutinib (AST2818 or Furmonertinib). AST2818 is the subject of clinical trial NCT03787992 into the clinical efficacy thereof in NSCLC.

In certain aspects, the third generation EGFR tyrosine kinase inhibitor is Rezivertinib (BPI-7711) which is an orally active, selective and irreversible third-generation EGFR tyrosine kinase inhibitor (TKI). Rezivertinib is the subject of clinical trial NCT03866499 into the clinical efficacy thereof in NSCLC.

In certain aspects, the third generation EGFR tyrosine kinase inhibitor is Avitinib (Abivertinib/AC0010)), which is a pyrrolopyrimidine-based irreversible epidermal growth factor receptor (EGFR) inhibitor with an IC50 of 7.68 nM. Avitinib is the subject of clinical trial NCT03856697 into the clinical efficacy thereof in NSCLC.

In certain aspects, the third generation EGFR tyrosine kinase inhibitor is ASK120067 which is a potent and orally active inhibitor of EGFR. ASK120067 is a third-generation EGFR-TKI for the research of non-small cell lung cancer (NSCLC). ASK120067 is the subject of a clinical trial (NCT04143607) into the clinical efficacy thereof in NSCLC.

In certain aspects, the third generation EGFR tyrosine kinase inhibitor is Oritinib (SH-1028, Nanjing Sanhome Pharmaceutical Co., Ltd., Nanjing, China). SH-1028 is the subject of clinical trial NCT04239833 into the clinical efficacy thereof in NSCLC.

In certain aspects, the third generation EGFR tyrosine kinase inhibitor is Rociletinib (CO-1686), which is an orally delivered kinase inhibitor that specifically targets the mutant EGFR forms. Rociletinib was the subject of clinical trial NCT02186301 into the clinical efficacy thereof in NSCLC.

In certain aspects, the third generation EGFR tyrosine kinase inhibitor is Olmutinib (HM61713; BI-1482694) which is an orally active and irreversible third generation EGFR tyrosine kinase inhibitor that binds to a cysteine residue near the kinase domain. Olmutinib can be used in the research into NSCLC. Olmutinib was the subject of clinical trial NCT02485652 into the clinical efficacy thereof in NSCLC.

In certain aspects, the third generation EGFR tyrosine kinase inhibitor is Nazartinib (EGF816), which is a third-generation EGFR TKI that selectively inhibits EGFR activating mutations, in patients with advanced EGFR-mutant NSCLC. Nazartinib was the subject of clinical trial NCT03529084 into the clinical efficacy thereof in NSCLC.

In certain aspects, the third generation EGFR tyrosine kinase inhibitor is Naquotinib, which is an orally available, irreversible, third-generation, mutant-selective, epidermal growth factor receptor (EGFR) inhibitor. Naquotinib was the subject of clinical trial NCT02588261 into the clinical efficacy thereof in NSCLC.

In certain aspects, the third generation EGFR tyrosine kinase inhibitor is Mavelertinib (PF-0647775), which is a selective, orally available and irreversible EGFR tyrosine kinase inhibitor (EGFR TKI). Mavelertinib was the subject of clinical trial NCT02349633 into the clinical efficacy thereof in NSCLC.

cMET, also called tyrosine-protein kinase MET or hepatocyte growth factor receptor (HGFR), is a protein that in humans is encoded by the MET gene. The protein possesses tyrosine kinase activity. The primary single chain precursor protein is post-translationally cleaved to produce the alpha and beta subunits, which are disulfide linked to form the mature receptor.

Dysregulation of, or aberrantly activated cMET may induce tumor growth, the formation of new blood vessels (angiogenesis) that supply the tumor with nutrients, and cancer spread to other organs (metastasis). cMET is deregulated in many types of human malignancies, including cancers of kidney, liver, stomach, breast, and brain. The cMET gene is known under a number of different names such as MET Proto-Oncogene, Receptor Tyrosine Kinase; Hepatocyte Growth Factor Receptor; Tyrosine-Protein Kinase Met; Scatter Factor Receptor; Proto-Oncogene C-Met; HGF/SF Receptor; HGF Receptor; SF Receptor; EC 2.7.10.1; Met Proto-Oncogene; EC 2.7.10; DFNB97; AUTS9; RCCP2; C-Met; MET; HGFR; External Ids for cMET are HGNC: 7029; Entrez Gene: 4233; Ensembl: ENSG00000105976; OMIM: 164860 and UniProtKB: P08581. The accession numbers are primarily given to provide a further method of identification of cMET protein as a target, the actual sequence of the cMET protein bound by an antibody may vary, for instance because of a mutation in the encoding gene such as those occurring in some cancers or the like. Where reference herein is made to cMET, the reference refers to human cMET unless otherwise stated. The antigen-binding site that binds cMET, binds cMET and a variety of variants thereof such as those expressed on some cMET positive tumors. Examples of cMET aberrations or dysregulation include cMET mutations (such as an exon14 skipping mutation), cMET amplification, cMET overexpression, increased signaling of the cMET pathway, cMET gene amplification, and/or increased cMET protein activity. A cMET aberration can also be caused by increased HGF expression. Dysregulation c-MET is an established driver of tumor invasion, angiogenesis, and metastasis (Birchmeier et al., 2003). Three types of biological alterations of c-MET can lead to oncogenesis: amplification, mutation and fusion. These genomic alterations are found principally as either primary or secondary drivers of tumor growth and such aberrations have been reported to occur after treatment of cancer patients with EGFR tyrosine kinase inhibitors (cf. Suzawa et al., DOI: 10.1200/PO.19.00011 JCO Precision Oncology—May 10, Vol 3, 2019).

In certain aspects, the subject of the present disclosure under iv) has not received prior anticancer treatment. In certain aspects, said subject has not received prior treatment with a tyrosine kinase inhibitor. In certain aspects, said subject has not received prior treatment with a cMET inhibitor. In certain aspects, said subject has not received prior treatment with cMET a tyrosine kinase inhibitor. In certain aspects, said subject has not received prior treatment with a tyrosine kinase inhibitor such as capmatinib, tepotinib or savolitinib. Said subject may have received treatment with a chemotherapeutic agent (such as platinum-based chemotherapy) or an immunotherapeutic agent (such as pembrolizumab, nivolumab, cetuximab). In certain aspects, said subject has a metastatic non-small lung cancer. In certain aspects, said metastatic NSCLC have a mutation that leads to cMET exon 14 skipping. Exon 14 skipping may be detected by an FDA-approved test.

In certain aspects, said subject comprises a cMET exon 14 skipping mutation or a cancer which comprises a cMET exon 14 skipping mutation. Said subject has not received prior anti-cancer treatment for any cancer, such as an EGFR and/or cMET positive cancer or for a cancer comprising an EGFR and/or cMET aberration. In certain aspects, administration of or treatment with the bispecific antibody of the present disclosure is thus first line treatment. In certain aspects, said first line treatment is to prevent resistance to an EGFR and/or cMET tyrosine kinase treatment mechanism to develop, such as in a lung cancer patient or in a lung cancer, in particular in non-small cell lung cancer. Resistance mechanisms to EGFR and/or cMET tyrosine kinase inhibitors are well known to develop, especially in subjects with non-small cell lung cancer, such as in a metastatic or advanced cancer. Hence, the present disclosure also provides a bispecific antibody of the present disclosure for use in a method of preventing of a cancer having an EGFR and/or cMET tyrosine kinase inhibitor resistance from developing or occurring in a subject.

In certain aspects, the subject of the present disclosure under iv) is a human subject.

In certain aspects, the use or treatment comprises providing said subject under iv) with a dose of 1000, 1500 or 2000 mg of the bispecific antibody of the present disclosure. In certain aspects, said bispecific antibody is provided once every week or once every two weeks. In certain aspects, the use or treatment comprises providing said subject with a dose of 1500 mg of said bispecific antibody once every two weeks. The disclosure also provides a method of treatment of said subject that has a tumor, the method comprising administering the bispecific antibody as described herein to said subject as an individual in need thereof. Typically, said individual is one suffering from a disease involving aberrant cells, for examples said individual may be suffering from a tumor or a cancer.

In certain aspects, said subject under iv) has a cancer which is non-small cell lung cancer (NSCLC), head and neck cancer, gastric cancer, esophageal cancer, gastric/esophageal junction cancer, breast cancer, colon cancer, pancreatic cancer, ovarian cancer, colorectal cancer or bladder cancer. In certain aspects, said subject under iv) has a cancer which is non-small cell lung cancer (NSCLC).

In certain aspects, said subject under iv) has an advanced or metastatic cancer, such as advanced or metastatic NSCLC.

In certain aspects, treatment of said subject under iv) comprises a diagnostic step for assessing whether said cancer is an EGFR positive and/or cMET positive cancer or assessing the presence of an EGFR and/or cMET aberration in said cancer.

An antibody typically recognizes only a part of an antigen. The antigen is typically but not necessarily a protein. The recognition or binding site on an antigen, bound by an antibody is referred to as the epitope, where an epitope may be linear or conformational. Binding of an antibody to an antigen is typically specific. The ‘specificity’ of an antibody refers to its selectivity for a particular epitope, whereas ‘affinity’ refers to the strength of the interaction between the antibody's antigen binding site and the epitope it binds.

Exemplary antibodies of the present disclosure bind to EGFR and cMET, in certain aspects human EGFR and human cMET. An EGFR/cMET bispecific antibody of the disclosure binds to EGFR and, under otherwise identical conditions, at least 100-fold less to the homologous receptors ErbB-2 and ErbB-4 of the same species. An EGFR/cMET bispecific antibody of the disclosure binds to cMET and, under otherwise identical conditions, at least 100-fold less to the receptors ErbB-2 and ErbB-4 of the same species. Considering that the receptors are cell surface receptors, the binding may be assessed on cells that express the receptor(s). A bispecific antibody of the disclosure in certain aspects binds to human, cynomolgus EGFR and/or to mouse EGFR.

An antibody that binds EGFR and cMET may bind other proteins as well if such other proteins contain the same epitope. Hence, the term “binding” does not exclude binding of the antibodies to another protein or protein(s) that contain the same epitope. Such binding is typically referred to as cross-reactivity. An EGFR/cMET bispecific antibody typically does not bind to other proteins than EGFR and/or cMET on the membrane of cells in a post-natal, in certain aspects adult human. An antibody according to the disclosure is typically capable of binding EGFR with a binding affinity (i.e. equilibrium dissociation constant Kd) of at least 1×10e-6 M, as outlined in more detail below.

The term “antibody” as used herein means a proteinaceous molecule in certain aspects belonging to the immunoglobulin class of proteins. An antibody typically contains two variable domains that bind an epitope on an antigen. Such domains are derived from or share sequence homology with the variable domain of an antibody. A bispecific antibody of the disclosure in certain aspects comprises two variable domains. Antibodies for therapeutic use are in certain aspects as close to natural antibodies of the subject to be treated as possible (for instance human antibodies for human subjects). Antibody binding can be expressed in terms of specificity and affinity. The specificity determines which antigen or epitope thereof is specifically bound by the binding domain. Typically, antibodies for therapeutic applications can have affinities of up to 1×10e-10 M or higher. Antibodies such as bispecific antibodies of the disclosure in certain aspects comprise the constant domains (Fc part) of a natural antibody. An antibody of the disclosure is typically a bispecific full length antibody, in certain aspects of the human IgG subclass. In certain aspects, the antibodies of the present disclosure are of the human IgG1 subclass. Such antibodies of the disclosure can have good ADCC properties, have a favorable half-life upon in vivo administration to humans and CH3 engineering technology exists that can provide for modified heavy chains that preferentially form hetero-dimers over homo-dimers upon co-expression in clonal cells. ADCC activity of an antibody can also be improved through techniques known to persons of skill in the art.

In certain aspects, an antibody of the present disclosure is a “full length” antibody. The term ‘full length’ as disclosed herein is defined as comprising an essentially complete antibody, which however does not necessarily have all functions of an intact antibody. For the avoidance of doubt, a full length antibody contains two heavy and two light chains. Each chain contains constant (C) and variable (V) regions, which can be broken down into domains designated CH1, CH2, CH3, VH, and CL, VL. Typically, an antibody binds to antigen via the variable domains contained in the Fab portion, and after binding can interact with molecules and cells of the immune system through the constant domains, mostly through the Fc portion. Full length antibodies according to the disclosure encompasses antibodies wherein mutations may be present that provide desired characteristics. Antibodies wherein one or several amino acid residues are deleted, without essentially altering the specificity and/or affinity characteristics of the resulting antibody are embraced within the term “full length antibody”. For instance, an IgG antibody can have 1-20 amino acid residue insertions, deletions, or substitutions or a combination thereof in the constant region.

In certain aspects, an antibody of the present disclosure is a bispecific IgG antibody, such as a bispecific full length IgG1 antibody or a human IgG1. Full length IgG antibodies are preferred because of their typically favorable half-life and the desire to stay as close to fully autologous (human) molecules for reasons of immunogenicity. In certain aspects, an antibody of the disclosure is a full length IgG1, a full length IgG2, a full length IgG3 or a full length IgG4 antibody.

The variable domain that can bind EGFR and that comprises the amino acid sequence of the MF3370 or variant thereof as indicated herein, binds to EGFR domain III in certain aspects (see table 4 of international patent application PCT/NL2015/050124; WO2015/130172 which is incorporated by reference herein). In certain aspects, the variable domain blocks the binding of the ligand EGF to EGFR or competes with the EGF ligand for binding to EGFR. The binding of the variable domain to EGFR can be inhibited by cetuximab. The variable domain binds an epitope that is different from the epitope that is recognized by cetuximab and zalutumumab. For example, the variable domain binds to mouse EGFR whereas cetuximab and zalutumumab do not, indicating that one or more of the residues that differ between mouse and human EGFR domain III play a role in cetuximab and zalutumumab binding, but not in an antibody of the disclosure described herein. An advantage of a bispecific antibody of the disclosure having human, mouse, cynomolgus EGFR cross-reactivity is that it permits the use of xenograft studies with human cancer models, which may be more predictive with respect to effectivity and toxicity as the antibody also binds to the normal mouse cells that have the receptor, while also being capable of use in cynomolgus toxicology studies. In certain aspects the disclosure provides a bispecific antibody that comprises a first variable domain that can bind an extracellular part of human epidermal growth factor receptor (EGFR) and a second variable domain that can bind an extracellular part of human MET Proto-Oncogene, Receptor Tyrosine Kinase (cMET), wherein said first variable domain can also bind mouse EGFR, cynomolgus EGFR or both.

In certain aspects, a cMET variable domain comprises an amino acid sequence of the MF4356 or variant thereof as indicated herein, and in certain aspects blocks the binding of the antibody MetMab to cMET. A cMET variable domain in certain aspects comprises an amino acid sequence of the MF8230 or variant thereof as indicated herein, and in certain aspects blocks the binding of the antibody MetMab to cMET. The variable domain in certain aspects blocks the binding of the ligand HGF to cMET or competes with the ligand HGF for binding to cMET. The variable domain blocks the binding of the antibody MetMab to cMET when the binding of MetMab to cMET at half-maximum binding conditions is reduced by at least 40% and in certain aspects at least 60% in the presence of a saturating amount of said variable domain. The variable domain is in certain aspects provided in the context of a bivalent monospecific antibody. The cMET variable domain can in certain aspects bind the sema domain of cMET. The cMET variable domain of the disclosure may compete with 5D5 for binding cMET or not compete with reported anti-cMET reference antibodies, such as 5D5. See Table 2.

A variable domain of the present disclosure can bind EGFR (the first variable domain) and in certain aspects comprises a heavy chain variable region with a CDR1 sequence SYGIS; a CDR2 sequence WISAYX₁X₂NTNYAQKLQG and a CDR3 comprising the sequence X₃X₄X₅X₆HWWLX₇A wherein X₁=N or S; X₂=A or G; X₃=D or G; X₄=R, S or Y; X₅=H, L or Y; X₆=D or W and X₇=D or G.
X₁-7 is in certain aspects:
X₁=N; X₂=G; X₃=D; X₄═S; X₅=Y; X₆=W and X₇=G;
X₁=N; X₂=A; X₃=D; X₄═S; X₅=Y; X₆=W and X₇=G;
X₁═S; X₂=G; X₃=D; X₄═S; X₅=Y; X₆=W and X₇=G;
X₁=N; X₂=G; X₃=D; X₄=R; X₅=H; X₆=W and X₇=D;
X₁=N; X₂=A; X₃=D; X₄=R; X₅=H; X₆=W and X₇=D;
X₁═S; X₂=G; X₃=D; X₄=R; X₅=H; X₆=W and X₇=D;
X₁=N; X₂=G; X₃=G; X₄=Y; X₅=L; X₆=D and X₇=G;
X₁=N; X₂=A; X₃=G; X₄=Y; X₅=L; X₆=D and X₇=G; or
X₁═S; X₂=G; X₃=G; X₄=Y; X₅=L; X₆=D and X₇=G.

In certain aspects
X₁=N; X₂=G; X₃=D; X₄=R; X₅=H; X₆=W and X₇=D;
X₁=N; X₂=A; X₃=D; X₄=R; X₅=H; X₆=W and X₇=D; or
X₁═S; X₂=G; X₃=D; X₄=R; X₅=H; X₆=W and X₇=D.

In certain aspects X₁=N; X₂=G; X₃=D; X₄=R; X₅=H; X₆=W and X₇=D.

The amino acids following the amino acid A in the sequence X₃X₄X₅X₆HWWLX₇A in the CDR3 sequence of the first variable domain can vary. The amino acid sequence following the sequence X₃X₄X₅X₆HWWLX₇A can be FDY. The CDR3 of the first variable domain in certain aspects comprises the sequence X₃X₄X₅X₆HWWLX₇AF, in certain aspects X₃X₄X₅X₆HWWLX₇AFD, in certain aspects X₃X₄X₅X₆HWWLX₇AFDY.

The first variable domain in certain aspects comprises a heavy chain variable region with a CDR1 sequence SYGIS; a CDR2 sequence WISAYNGNTNYAQKLQG and a CDR3 sequence X₃X₄X₅X₆HWWLX₇A.

The first variable domain in certain aspects comprises a heavy chain variable region with a CDR1 sequence SYGIS; a CDR2 sequence WISAYNGNTNYAQKLQG and a CDR3 comprising the sequence DRHWHWWLDA. The amino acids following the sequence LDA in the CDR3 sequence of the first variable domain can vary. The amino acid sequence following the sequence LDA can be FDY. The CDR3 of the first variable domain in certain aspects comprises the sequence DRHWHWWLDAF, in certain aspects DRHWHWWLDAFD, in certain aspects DRHWHWWLDAFDY.

The first variable domain in certain aspects comprises a heavy chain variable region with the amino acid sequence of MF3353; MF8229; MF8228; MF3370; MF8233; MF8232; MF3393; MF8227 or MF8226 as depicted in FIG. 2 having at most 10, in certain aspects 0, 1, 2, 3, 4, 5, 6, 7, 8, 9 or 10 and in certain aspects having 0, 1, 2, 3, 4 or 5 amino acid insertions, deletions, substitutions or a combination thereof with respect to the indicated sequence. In certain aspects the first variable domain comprises a heavy chain variable region with the amino acid sequence of MF3353; MF8229; MF8228; MF3370; MF8233; MF8232; MF3393; MF8227 or MF8226 as depicted in FIG. 2. In certain aspects, the first variable domain comprises a heavy chain variable region with the CDR1, CDR2, and CDR3 amino acid sequence of MF3353; MF8229; MF8228; MF3370; MF8233; MF8232; MF3393; MF8227 or MF8226 as depicted in FIG. 2.

The variable domain that can bind cMET (the second variable domain) in certain aspects comprises a heavy chain variable region that comprises the amino acid sequence of one of the sequences of SEQ ID NO: 1-23 (FIG. 3) with 0-10, in certain aspects 0-5 amino acid insertions, deletions, substitutions, additions or a combination thereof. The heavy chain variable region of the second variable domain in certain aspects comprises the amino acid sequence of one of the sequences of SEQ ID NO: 1-3; 7; 8; 10; 13; 15; 16; 17; 21; 22 or 23 with 0-10 in certain aspects 0-5 amino acid insertions, deletions, substitutions, additions or a combination thereof. The heavy chain variable region of the second variable domain in certain aspects comprises the amino acid sequence of one of the sequences of SEQ ID NO: 2; 7; 8; 10; 13 or 23 with 0-10 in certain aspects 0-5 amino acid insertions, deletions, substitutions, additions or a combination thereof. The heavy chain variable region of the second variable domain in certain aspects comprises the amino acid sequence of the sequence of SEQ ID NO: 13 or SEQ ID NO: 23 with 0-10, in certain aspects 0-5 amino acid insertions, deletions, substitutions, additions or a combination thereof. In certain aspects, the second variable domain comprises a heavy chain variable region with the CDR1, CDR2, and CDR3 amino acid sequence of MF8225 (SEQ ID NO: 1), MF8243 (SEQ ID NO: 2), MF8224 (SEQ ID NO:3), MF8239 (SEQ ID NO: 4), MF8242 (SEQ ID NO: 5), MF8237 (SEQ ID NO: 6), MF8240 (SEQ ID NO: 7), MF8234 (SEQ ID NO: 8), MF8245 (SEQ ID NO: 9), MF8231 (SEQ ID NO: 10), MF8247 (SEQ ID NO: 11), MF8238 (SEQ ID NO: 12), MF8230 (SEQ ID NO: 13), MF8248 (SEQ ID NO: 14), MF8246 (SEQ ID NO: 15), MF8223 (SEQ ID NO: 16), MF8222 (SEQ ID NO: 17), MF8235 (SEQ ID NO: 18), MF8236 (SEQ ID NO: 19), MF8241 (SEQ ID NO: 20), MF8244 (SEQ ID NO: 21), MF8221 (SEQ ID NO: 22), or MF4356 (SEQ ID NO: 23).

In certain aspects the first variable domain comprises a heavy chain variable region with a CDR1 sequence SYGIS; a CDR2 sequence WISAYNGNTNYAQKLQG and a CDR3 comprising the sequence DRHWHWWLDA, in certain aspects DRHWHWWLDAFDY and wherein the second variable domain comprises a heavy chain variable region with a CDR1 sequence SYSMN; a CDR2 sequence WINTYTGDPTYAQGFTG and a CDR3 sequence ETYYYDRGGYPFDP. The CDR1, CDR2 and CDR3 of a light chain of the first and second variable domain in certain aspects comprises respectively the amino acid sequence CDR1-QSISSY, CDR2-AAS, CDR3-QQSYSTPPT, i.e. the CDRs of IGKV1-39 (according to IMGT).

In certain aspects the first variable domain comprises a heavy chain variable region with a CDR1 sequence SYGIS; a CDR2 sequence WISAYNGNTNYAQKLQG and a CDR3 comprising the sequence DRHWHWWLDA and wherein the second variable domain comprises a heavy chain variable region with a CDR1 sequence TYSMN; a CDR2 sequence WINTYTGDPTYAQGFTG and a CDR3 comprising the sequence ETYFYDRGGYPFDP. The CDR1, CDR2 and CDR3 of a light chain of the first and second variable domain in certain aspects comprises respectively the amino acid sequence CDR1-QSISSY, CDR2-AAS, CDR3-QQSYSTPPT, i.e. the CDRs of IGKV1-39 (according to IMGT).

A bispecific antibody that comprises a first variable domain that can bind an extracellular part of EGFR and a second variable domain that can bind an extracellular part of cMET wherein the first variable domain comprises a heavy chain variable region with a CDR1 sequence SYGIS; a CDR2 sequence WISAYNANTNYAQKLQG and a CDR3 comprising the sequence DRHWHWWLDA and wherein the second variable domain comprises a heavy chain variable region with a CDR1 sequence SYSMN; a CDR2 sequence WINTYTGDPTYAQGFTG and a CDR3 sequence ETYYYDRGGYPFDP. The CDR1, CDR2 and CDR3 of a light chain of the first and second variable domain in certain aspects comprises respectively the amino acid sequence CDR1-QSISSY, CDR2-AAS, CDR3-QQSYSTPPT, i.e. the CDRs of IGKV1-39 (according to IMGT).

A bispecific antibody that comprises a first variable domain that can bind an extracellular part of EGFR and a second variable domain that can bind an extracellular part of cMET wherein the first variable domain comprises a heavy chain variable region with a CDR1 sequence SYGIS; a CDR2 sequence WISAYNANTNYAQKLQG and a CDR3 comprising the sequence DRHWHWWLDA and wherein the second variable domain comprises a heavy chain variable region with a CDR1 sequence TYSMN; a CDR2 sequence WINTYTGDPTYAQGFTG and a CDR3 comprising the sequence ETYFYDRGGYPFDP. The CDR1, CDR2 and CDR3 of a light chain of the first and second variable domain in certain aspects comprises respectively the amino acid sequence CDR1-QSISSY, CDR2-AAS, CDR3-QQSYSTPPT, i.e. the CDRs of IGKV1-39 (according to IMGT).

A bispecific antibody that comprises a first variable domain that can bind an extracellular part of EGFR and a second variable domain that can bind an extracellular part of cMET wherein the first variable domain comprises a heavy chain variable region with a CDR1 sequence SYGIS; a CDR2 sequence WISAYSGNTNYAQKLQG and a CDR3 comprising the sequence DRHWHWWLDA and wherein the second variable domain comprises a heavy chain variable region with a CDR1 sequence SYSMN; a CDR2 sequence WINTYTGDPTYAQGFTG and a CDR3 sequence ETYYYDRGGYPFDP. The CDR1, CDR2 and CDR3 of a light chain of the first and second variable domain in certain aspects comprises respectively the amino acid sequence CDR1-QSISSY, CDR2-AAS, CDR3-QQSYSTPPT, i.e. the CDRs of IGKV1-39 (according to IMGT).

A bispecific antibody that comprises a first variable domain that can bind an extracellular part of EGFR and a second variable domain that can bind an extracellular part of cMET wherein the first variable domain comprises a heavy chain variable region with a CDR1 sequence SYGIS; a CDR2 sequence WISAYSGNTNYAQKLQG and a CDR3 comprising the sequence DRHWHWWLDA and wherein the second variable domain comprises a heavy chain variable region with a CDR1 sequence TYSMN; a CDR2 sequence WINTYTGDPTYAQGFTG and a CDR3 comprising the sequence ETYFYDRGGYPFDP. The CDR1, CDR2 and CDR3 of a light chain of the first and second variable domain in certain aspects comprises respectively the amino acid sequence CDR1-QSISSY, CDR2-AAS, CDR3-QQSYSTPPT, i.e. the CDRs of IGKV1-39 (according to IMGT).

In certain aspects wherein a cMET binding variable domain is described to have a CDR2 sequence “WINTYTGDPTYAQGFTG” the CDR2 sequence can also be “WINTYTGDPTYAQGFT”.

The CDR1, CDR2 and CDR3 of a light chain of the first and second variable domain as described herein in certain aspects comprises respectively the amino acid sequence CDR1-QSISSY, CDR2-AAS, CDR3-QQSYSTPPT, i.e. the CDRs of IGKV1-39 (according to IMGT). In some of such aspects, the CDR3 comprises the amino acid sequence QQSYSTP. In certain aspects of a bispecific antibody as described herein the first and second variable domain comprise a common light chain, in certain aspects a light chain variable domain of FIG. 4B.

In certain aspects, an EGFR/cMET bispecific antibody comprises a first variable domain that can bind an extracellular part of human EGFR that comprises the CDR1, CDR2 and CDR3 of the heavy chain variable region of MF3755 depicted in FIG. 1 and a second variable domain that can bind an extracellular part of human cMET that comprises the CDR1, CDR2 and CDR3 of the heavy chain variable region of MF4297 depicted in FIG. 1. The light chain variable region in said first and second variable domain is in certain aspects a common light chain variable region as described herein. The CDR1, CDR2 and CDR3 of a light chain of the first and second variable domain in certain aspects comprises respectively the amino acid sequence CDR1-QSISSY, CDR2-AAS, CDR3-QQSYSTPPT, i.e. the CDRs of IGKV1-39 (according to IMGT). In certain aspects the antibody comprises a heavy chain variable region with the amino acid sequence of MF3755 as depicted in FIG. 1 having at most 10, in certain aspects 0, 1, 2, 3, 4, 5, 6, 7, 8, 9 or 10 and in certain aspects having 0, 1, 2, 3, 4 or 5 amino acid insertions, deletions, substitutions or a combination thereof with respect to the indicated sequence. In certain aspects the first variable domain comprises a heavy chain variable region with the amino acid sequence of MF3755 as depicted in FIG. 1. The variable domain that can bind cMET (the second variable domain) in certain aspects comprises a heavy chain variable region that comprises the amino acid sequence of MF4297 as depicted in FIG. 1 with 0-10 in certain aspects 0-5 amino acid insertions, deletions, substitutions, additions or a combination thereof. The heavy chain variable region of the second variable domain in certain aspects comprises the amino acid sequence of MF4297 as depicted in FIG. 1.

The term ‘bispecific’ (bs) in the context of the present disclosure means that an antibody is capable of binding two different targets or two epitopes on the same target, for example, where one variable domain of the antibody (as defined above) binds to an epitope on EGFR and a second variable domain binds to an epitope on cMET. Depending on the expression level, (sub-) cellular localization and stoichiometry of the two antigens recognized by a bispecific antibody, both Fab arms of the antibody may or may not simultaneously bind their epitope. One arm of the bispecific antibody typically contains the variable domain of one antibody and the other arm contains the variable domain of another antibody (i.e. one arm of the bispecific antibody is formed by one heavy chain paired with one light chain whereas the other arm is formed by a different heavy chain paired with a light chain). Thus, in certain aspects, the stoichiometry of a bispecific antibody of the disclosure is 1:1, EGFR:cMET binding.

The heavy chain variable regions of the bispecific antibody of the disclosure are typically different from each other, whereas the light chain variable regions are in certain aspects the same. A bispecific antibody wherein the different heavy chain variable regions are associated with the same light chain variable region is also referred to as a bispecific antibody with a common light chain variable region (cLcv). It is preferred that the light chain constant region is also the same. Such bispecific antibodies are referred to as having a common light chain (cLc). Further provided is therefore a bispecific antibody according to the disclosure, wherein both arms comprise a common light chain.

The term ‘common light chain’ according to the present disclosure refers to two or more light chains in a bispecific antibody which may be identical or have some amino acid sequence differences while the binding specificity of the full length antibody is not affected. It is for instance possible within the scope of the definition of common light chains as used herein, to prepare or find light chains that are not identical but still functionally equivalent, e.g., by introducing and testing conservative amino acid changes, changes of amino acids in regions that do not or only partly contribute to binding specificity when paired with the heavy chain, and the like. The terms ‘common light chain’, ‘common LC’, ‘CLC’, ‘single light chain’ with or without the addition of the term ‘rearranged’ are all used herein interchangeably. The terms ‘common light chain variable region’, ‘common VL’, ‘common LCv’, ‘CLCv’, ‘single VL’ with or without the addition of the term ‘rearranged’ are all used herein interchangeably. In certain aspects, a bispecific antibody of the present disclosure has a common light chain (variable region) that can combine with at least two, and in certain aspects a plurality of heavy chains (variable regions) of different binding specificity to form antibodies with functional antigen binding domains (e.g., WO2009/157771). The common light chain (variable region) is in certain aspects a human light chain (variable region). A common light chain (variable region) in certain aspects has a germline sequence. In certain aspects, a germline sequence is a light chain variable region that has good thermodynamic stability, yield and solubility. In certain aspects, a germline light chain is O12. A common light chain in certain aspects comprises the light chain encoded by a germline human Vk gene segment, and is in certain aspects the rearranged germline human kappa light chain IgVκ1-39*01/IGJK1*01 (FIG. 4A). The common light chain variable region is in certain aspects the variable region of the rearranged germline human kappa light chain IgVκ1-39*01/IGJK1*01. A common light chain in certain aspects comprises a light chain variable region as depicted in FIG. 4B, or 4d with 0-5 amino acid insertions, deletions, substitutions, additions or a combination thereof. The common light in certain aspects further comprises a light chain constant region, in certain aspects a kappa light chain constant region. A nucleic acid that encodes the common light chain can be codon optimized for the cell system used to express the common light chain protein. The encoding nucleic acid can deviate from a germ-line nucleic acid sequence.

In certain aspects the light chain comprises a light chain region comprising the amino acid sequence of an O12/IgVκ1-39*01 gene segment as depicted in FIG. 4A with 0-10, in certain aspects 0-5 amino acid insertions, deletions, substitutions, additions or a combination thereof. The phrase “O12 light chain” will be used throughout the specification as short for “a light chain comprising a light chain variable region comprising the amino acid sequence of an O12/IgVκ1-39*01 gene segment as part of depicted FIG. 4A with 0-10, in certain aspects 0-5 amino acid insertions, deletions, substitutions, additions or a combination thereof. IgVκ1-39 is short for Immunoglobulin Variable Kappa 1-39 Gene. The gene is also known as Immunoglobulin Kappa Variable 1-39; IGKV139; IGKV1-39; O12a or O12. External Ids for the gene are HGNC: 5740; Entrez Gene: 28930; Ensembl: ENSG00000242371. In certain aspects, an amino acid sequence for IgVκ1-39 is given in FIG. 4E. This lists the sequence of the V-region. The V-region can be combined with one of five J-regions. FIGS. 4B and 4d describe two preferred sequences for IgVκ1-39 in combination with a J-region. The joined sequences are indicated as IGKV1-39/jk1 and IGKV1-39/jk5; alternative names are IgVκ1-39*01/IGJK1*01 or IgVκ1-39*01/IGJK5*01 (nomenclature according to the IMGT database worldwide web at imgt.org).

It is preferred that the O12/IgVκ1-39*01 comprising light chain variable region is a germline sequence. It is further preferred that the IGJK1*01 or/IGJK5*01 comprising light chain variable region is a germline sequence. In certain aspects, the IGKV1-39/jk1 or IGKV1-39/jk5 light chain variable regions are germline sequences.

In certain aspects the light chain variable region comprises a germline O12/IgVκ1-39*01. In certain aspects the light chain variable region comprises the kappa light chain IgVκ1-39*01/IGJK1*01 or IgVκ1-39*01/IGJK5*01. In certain aspects a IgVκ1-39*01/IGJK1*01. The light chain variable region in certain aspects comprises a germline kappa light chain IgVκ1-39*01/IGJK1*01 or germline kappa light chain IgVκ1-39*01/IGJK5*01, in certain aspects a germline IgVκ1-39*01/IGJK1*01.

Mature B-cells that produce an antibody with an O12 light chain often produce a light chain that has undergone one or more mutations with respect to the germline sequence, i.e. the normal sequence in non-lymphoid cells of the organism. The process that is responsible for these mutations is often referred to as somatic (hyper) mutation. The resulting light chain is referred to as an affinity matured light chain. Such light chains, when derived from an O12 germline sequence are O12-derived light chains. In this specification, the phrase “common light chain” will include “common light chain derived light chains and the phrase “O12 light chains” will include O12-derived light chains. The mutations that are introduced by somatic hypermutation can also be introduced artificially in the lab. In the lab also other mutations can be introduced without affecting the properties of the light chain in kind, not necessarily in amount. A light chain is at least an O12 light chain if it comprises a sequence as depicted in FIG. 4A, FIG. 4B; FIG. 4D or FIG. 4E with 0-10, in certain aspects 0-5 amino acid insertions, deletions, substitutions, additions or a combination thereof. In certain aspects the O12 light chain is a light chain comprising a sequence as depicted in FIG. 4A; 4b; 4d or 4e with 0-9, 0-8, 0-7, 0-6, 0-5, 0-4 amino acid insertions, deletions, substitutions, additions or a combination thereof. In certain aspects the O12 light chain is a light chain comprising a sequence as depicted in FIG. 4A, FIG. 4B; FIG. 4D or FIG. 4E with 0-5, in certain aspects 0-4, in certain aspects 0-3 amino acid insertions, deletions, substitutions, additions or a combination thereof. In certain aspects the O12 light chain is a light chain comprising a sequence as depicted in FIG. 4A, FIG. 4B; FIG. 4D or FIG. 4E with 0-2, in certain aspects 0-1, in certain aspects 0 amino acid insertions, deletions, substitutions, additions or a combination thereof. In certain aspects the O12 light chain is a light chain comprising a sequence as depicted in FIG. 4A or FIG. 4B with the mentioned amino acid insertions, deletions, substitutions, additions or a combination thereof. In certain aspects the light chain comprises the sequence of FIG. 4A. In certain aspects the light chain variable region comprises the sequence of FIG. 4B. The mentioned 1, 2, 3, 4 or 5 amino acid substitutions are in certain aspects conservative amino acid substitutions and may be present in the CDR regions of the heavy and/or light chain; the insertions, deletions, substitutions or combination thereof are in certain aspects not in the CDR3 region of the VL chain, in certain aspects not in the CDR1, CDR2 or CDR3 region or FR4 region of the VL chain.

The common light chain can have a lambda light chain and this is therefore also provided in the context of the disclosure, however a kappa light chain is preferred. The constant part of a common light chain of the disclosure can be a constant region of a kappa or a lambda light chain. It is in certain aspects a constant region of a kappa light chain, in certain aspects said common light chain is a germline light chain, in certain aspects a rearranged germline human kappa light chain comprising the IgVKl-39 gene segment, in certain aspects the rearranged germline human kappa light chain IgVKl-39*01/IGJKl*01 (FIG. 4). The terms rearranged germline human kappa light chain IgVκ1-39*01/IGJK1*01, IGKV1-39/IGKJ1, huVκ1-39 light chain or in short huVκ1-39, or simply 1-39 are used interchangeably throughout the application.

A cell that produces a common light chain can produce for instance rearranged germline human kappa light chain IgVκ1-39*01/IGJK1*01 and a light chain comprising the variable region of the mentioned light chain fused to a lambda constant region.

In certain aspects the light chain variable region comprises the amino acid sequence DIQMT QSPSS LSASV GDRVT ITCRA SQSIS SYLNW YQQKP GKAPK LLIYA ASSLQ SGVPS RFSGS GSGTD FTLTI SSLQP EDFAT YYCQQ SYSTP PTFGQ GTKVE IK or DIQMT QSPSS LSASV GDRVT ITCRA SQSIS SYLNW YQQKP GKAPK LLIYA ASSLQ SGVPS RESGS GSGTD FTLTI SSLQP EDFAT YYCQQ SYSTP PITFG QGTRL EIK with 0-10, in certain aspects 0-5 amino acid insertions, deletions, substitutions, additions or a combination thereof. In certain aspects the light chain variable region comprises 0-9, 0-8, 0-7, 0-6, 0-5, 0-4, in certain aspects 0-3, in certain aspects 0-2, in certain aspects 0-1 and in certain aspects 0 amino acid insertions, deletions, substitutions, additions with respect to the indicated amino acid sequence, or a combination thereof. A combination of an insertion, deletion, addition or substitution is a combination as claimed if aligned sequences do not differ at more than 5 positions. In certain aspects the light chain variable region comprises the amino acid sequence DIQMT QSPSS LSASV GDRVT ITCRA SQSIS SYLNW YQQKP GKAPK LLIYA ASSLQ SGVPS RFSGS GSGTD FTLTI SSLQP EDFAT YYCQQ SYSTP PTFGQ GTKVE IK or DIQMT QSPSS LSASV GDRVT ITCRA SQSIS SYLNW YQQKP GKAPK LLIYA ASSLQ SGVPS RFSGS GSGTD FTLTI SSLQP EDFAT YYCQQ SYSTP PITFG QGTRL EIK. In certain aspects the light chain variable region comprises the amino acid sequence DIQMT QSPSS LSASV GDRVT ITCRA SQSIS SYLNW YQQKP GKAPK LLIYA ASSLQ SGVPS RFSGS GSGTD FTLTI SSLQP EDFAT YYCQQ SYSTP PTFGQ GTKVE IK. In another aspect, the light chain variable region comprises the amino acid sequence DIQMT QSPSS LSASV GDRVT ITCRA SQSIS SYLNW YQQKP GKAPK LLIYA ASSLQ SGVPS RFSGS GSGTD FTLTI SSLQP EDFAT YYCQQ SYSTP PITFG QGTRL EIK.

The amino acid insertions, deletions, substitutions, additions or combination thereof are in certain aspects not in the CDR3 region of the light chain variable region, in certain aspects not in the CDR1 or CDR2 region of the light chain variable region. In certain aspects the light chain variable region does not comprise a deletion, addition or insertion with respect to the sequence indicated. In this embodiment the heavy chain variable region can have 0-5 amino acid substitutions with respect to the indicated amino acid sequence. An amino acid substitution is in certain aspects a conservative amino acid substitution. The CDR1, CDR2 and CDR3 of a light chain of an antibody of the disclosure in certain aspects comprises respectively the amino acid sequence CDR1-QSISSY, CDR2-AAS, CDR3-QQSYSTPPT, i.e. the CDRs of IGKV1-39 (according to IMGT). In certain aspects, said CDR3 sequence comprises QQSYSTPPT.

Bispecific antibodies as described herein in certain aspects have one heavy chain variable region/light chain variable region (VH/VL) combination that binds an extracellular part of EGFR and a second VH/VL combination that binds an extracellular of cMET. In certain aspects the VL in said first VH/VL combination is similar to the VL in said second VH/VL combination. In certain aspects, the VLs in the first and second VH/VL combinations are identical. In certain aspects, the bispecific antibody is a full length antibody which has one heavy/light (H/L) chain combination that binds an extracellular part of EGFR and one H/L chain combination that binds an extracellular part of cMET. In certain aspects the light chain in said first H/L chain combination is similar to the light chain in said second H/L chain combination. In certain aspects, the light chains in the first and second H/L chain combinations are identical.

Several methods have been published to produce a host cell whose expression favors the production of the bispecific antibody or vice versa, the monospecific antibodies. In the present disclosure it is preferred that the cellular expression of the antibody molecules is favored toward the production of the bispecific antibody over the production of the respective monospecific antibodies. Such is typically achieved by modifying the constant region of the heavy chains such that they favor heterodimerization (i.e. dimerization with the heavy chain of the other heavy/light chain combination) over homodimerization. In certain aspects the bispecific antibody of the disclosure comprises two different immunoglobulin heavy chains with compatible heterodimerization domains. Various compatible heterodimerization domains have been described in the art. The compatible heterodimerization domains are in certain aspects compatible immunoglobulin heavy chain CH3 heterodimerization domains. When wildtype CH3 domains are used, co-expression of two different heavy chains (A and B) and a common light chain will result in three different antibody species, AA, AB and BB. AA and BB are designations for the two mono-specific, bivalent antibodies, and AB is a designation for the bispecific antibody. To increase the percentage of the desired bispecific product (AB) CH3 engineering can be employed, or in other words, one can use heavy chains with compatible hetero-dimerization domains, as defined hereunder. The art describes various ways in which such hetero-dimerization of heavy chains can be achieved. One way is to generate ‘knob into hole’ bispecific antibodies.

The term ‘compatible hetero-dimerization domains’ as used herein refers to protein domains that are engineered such that engineered domain A′ will preferentially form heterodimers with engineered domain B′ and vice versa, homo-dimerization between A′-A′ and B′-B′ is diminished.

In U.S. Ser. No. 13/866,747 (now issued as U.S. Pat. No. 9,248,181), U.S. Ser. No. 14/081,848 (now issued as U.S. Pat. No. 9,358,286) and PCT/NL2013/050294 (published as WO2013/157954; incorporated herein by reference) methods and means are disclosed for producing bispecific antibodies using compatible heterodimerization domains. These means and methods can also be favorably employed in the present disclosure. Specifically, a bispecific antibody of the disclosure in certain aspects comprises mutations to produce substantial expression of bispecific full length IgG molecules in host cells. Preferred mutations are the amino acid substitutions L351K and T366K in the first CH3 domain (the ‘KK-variant’ heavy chain) and the amino acid substitutions L351D and L368E in the second domain (the ‘DE-variant’ heavy chain), or vice versa. U.S. Pat. Nos. 9,248,181 and 9,358,286 patents as well as the WO2013/157954 PCT application (which are incorporated by reference herein) demonstrate that the DE-variant and KK-variant preferentially pair to form heterodimers (so-called ‘DEKK’ bispecific molecules). Homodimerization of DE-variant heavy chains (DEDE homodimers) are disfavored due to repulsion between the charged residues in the CH3-CH3 interface between identical heavy chains.

Bispecific antibodies can be generated by (transient) transfection of plasmids encoding a light chain and two different heavy chains that are CH3 engineered to ensure efficient hetero-dimerization and formation of the bispecific antibodies. The production of these chains in a single cell leads to the favored formation of bispecific antibodies over the formation of monospecific antibodies. Preferred mutations to produce essentially only bispecific full length IgG1 molecules are amino acid substitutions at positions 351 and 366, e.g. L351K and T366K (numbering according to EU numbering) in the first CH3 domain (the ‘KK-variant’ heavy chain) and amino acid substitutions at positions 351 and 368, e.g. L351D and L368E in the second CH3 domain (the ‘DE-variant’ heavy chain), or vice versa.

In one embodiment the heavy chain/light chain combination that comprises the variable domain that binds EGFR, comprises a DE variant of the heavy chain. In this embodiment the heavy chain/light chain combination that comprises the variable domain that can bind to cMET comprises a KK variant of the heavy chain. The KK variant of the heavy chain that binds cMET do not produce homodimers thereby rendering the observed effect of HGF induced cMET activation inhibition by the bispecific antibody very precise. It avoids activation of cMET sometimes observed with bivalent cMET antibodies (agonism).

The Fc region mediates effector functions of an antibody, such as complement-dependent cytotoxicity (CDC), antibody-dependent cellular cytotoxicity (ADCC) and antibody-dependent cell phagocytosis (ADCP). Depending on the therapeutic antibody or Fc fusion protein application, it may be desired to either reduce or increase the effector function. Reduced effector function can be desired when an immune response is to be activated, enhanced or stimulated as in some of the embodiments of the disclosure. Antibodies with reduced effector functions can be used to target cell-surface molecules of immune cells, among others. In certain aspects, the antibody of the present disclosure promotes ACDP. In certain aspects, the antibody of the present disclosure promotes ADCC. In certain aspects, the antibody of the present disclosure promotes ADCC to a higher extent than amivantamab. In certain aspects, the antibody of the present disclosure promotes ADCC to a higher extent than amivantamab for cells or cancers having a cMET aberration including cMET amplification and/or a cMET exon 14 skipping mutation.

Antibodies with reduced effector functions are in certain aspects IgG antibodies comprising a modified CH2/lower hinge region, for instance to reduce Fc-receptor interaction or to reduce C1q binding. In certain aspects the antibody of the disclosure is an IgG antibody with a mutant CH2 and/or lower hinge domain such that interaction of the bispecific IgG antibody to a Fc-gamma receptor is reduced. An antibody comprising a mutant CH2 region is in certain aspects an IgG1 antibody. Such a mutant IgG1 CH2 and/or lower hinge domain in certain aspects comprise an amino substitution at position 235 and/or 236 (EU-numbering), in certain aspects an L235G and/or G236R substitution (FIG. 5D).

An antibody of the present disclosure in certain aspects has effector function. A bispecific antibody of the disclosure in certain aspects comprises antibody-dependent cell-mediated cytotoxicity (ADCC). The antibody can be engineered to enhance the ADCC activity (for review, see Cancer Sci. 2009 September; 100 (9): 1566-72. Engineered therapeutic antibodies with improved effector functions. Kubota T, Niwa R, Satoh M, Akinaga S, Shitara K, Hanai N). Several in vitro methods exist for determining the efficacy of antibodies or effector cells in eliciting ADCC. Among these are chromium-51 [Cr51] release assays, europium [Eu] release assays, and sulfur-35 [S35] release assays. Usually, a labeled target cell line expressing a certain surface-exposed antigen is incubated with antibody specific for that antigen. After washing, effector cells expressing Fc receptor CD16 are co-incubated with the antibody-labeled target cells. Target cell lysis is subsequently measured by release of intracellular label by a scintillation counter or spectrophotometry. In certain aspects a bispecific antibody of the disclosure exhibits ADCC activity. In such aspect the bispecific antibody can have improved ADCC activity. In such aspect the antibody can have altered ADCC activity by means of one or more CH2 mutations as described elsewhere herein and by techniques known to in the art. One technique for enhancing ADCC of an antibody is afucosylation. (See for instance Junttila, T. T., K. Parsons, et al. (2010). “Superior In vivo Efficacy of Afucosylated Trastuzumab in the Treatment of HER2-Amplified Breast Cancer.” Cancer Research 70 (11): 4481-4489). Further provided is therefore a bispecific antibody according to the disclosure, which is afucosylated. In certain aspects, the antibody of the present disclosure comprises two afucosylated CH2 domains. In certain aspects, the antibody of the present disclosure comprises a total of two CH2 domains, both of which are afucosylated. In certain aspects, the antibody of the present disclosure is a full-length antibody, such as of the IgG-type, having two CH2 domains, both of which are afucosylated. Alternatively, or additionally, multiple other strategies can be used to achieve ADCC enhancement, for instance including glycoengineering (Kyowa Hakko/Biowa, GlycArt (Roche) and Eureka Therapeutics) and mutagenesis, all of which seek to improve Fc binding to low-affinity activating FcγRIIIa, and/or to reduce binding to the low affinity inhibitory FcγRIIb. A bispecific antibody of the disclosure is in certain aspects afucosylated in order to enhance ADCC activity. A bispecific antibody of the disclosure in certain aspects comprises a reduced amount of fucosylation of the N-linked carbohydrate structure in the Fc region, when compared to the same antibody produced in a normal CHO cell.

A variant of an antibody or bispecific antibody as described herein comprises a functional part, derivative and/or analogue of the antibody or bispecific antibody. The variant maintains the binding specificity of the (bispecific) antibody. The functional part, derivative and/or analogue maintains the binding specificity of the (bispecific) antibody. Binding specificity is defined by capacity to bind an extracellular part of a first membrane protein and a second membrane protein as described herein.

A bispecific antibody of the present disclosure is in certain aspects used in humans. In certain aspects, an antibody of the disclosure is a human or humanized antibody, in certain aspects human. The constant region of a bispecific antibody of the disclosure is in certain aspects a human constant region. The constant region may contain one or more, in certain aspects not more than 10, in certain aspects not more than 5 amino-acid differences with the constant region of a naturally occurring human antibody. It is preferred that the constant part is entirely derived from a naturally occurring human antibody. Various antibodies produced herein are derived from a human antibody variable domain library. As such these variable domains are human. The unique CDR regions may be derived from humans, be synthetic or derived from another organism. The variable region is considered a humanized variable region when it has an amino acid sequence that is identical to an amino acid sequence of the variable region of a naturally occurring human antibody, but for the CDR regions. In such embodiments, the VH of a variable domain of an antibody that binds EGFR or cMET of the disclosure may contain one or more, in certain aspects not more than 10, in certain aspects not more than 5 amino-acid differences with the variable region of a naturally occurring human antibody, not counting possible differences in the amino acid sequence of the CDR regions. The light chain variable region of an EGFR binding domain and/or a cMET binding domain in an antibody of the disclosure may contain one or more, in certain aspects not more than 10, in certain aspects not more than 5 amino-acid differences with the variable region of a naturally occurring human antibody, not counting possible differences in the amino acid sequence of the CDR regions. The light chain in an antibody of the disclosure may contain one or more, in certain aspects not more than 10, in certain aspects not more than 5 amino-acid differences with the variable region of a naturally occurring human antibody, not counting possible differences in the amino acid sequence of the CDR regions. Such mutations also occur in nature in the context of somatic hypermutation.

Antibodies may be derived from various animal species, at least with regard to the heavy chain variable region. It is common practice to humanize such e.g. murine heavy chain variable regions. There are various ways in which this can be achieved among which there are CDR-grafting into a human heavy chain variable region with a 3D-structure that matches the 3-D structure of the murine heavy chain variable region; deimmunization of the murine heavy chain variable region, in certain aspects done by removing known or suspected T- or B-cell epitopes from the murine heavy chain variable region. The removal is typically by substituting one or more of the amino acids in the epitope for another (typically conservative) amino acid, such that the sequence of the epitope is modified such that it is no longer a T- or B-cell epitope.

Deimmunized murine heavy chain variable regions are less immunogenic in humans than the original murine heavy chain variable region. In certain aspects a variable region or domain of the disclosure is further humanized, such as for instance veneered. By using veneering techniques, exterior residues which are readily encountered by the immune system are selectively replaced with human residues to provide a hybrid molecule that comprises either a weakly immunogenic or substantially non-immunogenic veneered surface. An animal as used in the disclosure is in certain aspects a mammal, in certain aspects a primate, in certain aspects a human.

A bispecific antibody according to the present disclosure in certain aspects comprises a constant region of a human antibody. According to differences in their heavy chain constant domains, antibodies are grouped into five classes, or isotypes: IgG, IgA, IgM, IgD, and IgE. These classes or isotypes comprise at least one of said heavy chains that is named with a corresponding Greek letter. Certain aspects comprises an antibody wherein said constant region is selected from the group of IgG, IgA, IgM, IgD, and IgE constant regions, in certain aspects said constant region comprises an IgG constant region, i.e. selected from the group consisting of IgG1, IgG2, IgG3 and IgG4. In certain aspects, said constant region is an IgG1 or IgG4 constant region, in certain aspects a mutated IgG1 constant region. Some variation in the constant region of IgG1 occurs in nature and/or is allowed without changing the immunological properties of the resulting antibody. Variation can also be introduced artificially to install certain preferred features on the antibody or parts thereof. Such features are for instance described herein in the context of CH2 and CH3. Typically between about 1-10 amino acid insertions, deletions, substitutions or a combination thereof are allowed in the constant region.

A VH chain of FIG. 1, 2 or 3 in certain aspects has at most 15, in certain aspects 0, 1, 2, 3, 4, 5, 6, 7, 8, 9 or 10 amino acid insertions, deletions, substitutions or a combination thereof with respect to the VH chain depicted in FIG. 1, 2 or 3, in certain aspects has 0, 1, 2, 3, 4 or 5 amino acid insertions, deletions, substitutions or a combination thereof with respect to the VH chain depicted in FIG. 1, 2 or 3, in certain aspects 0, 1, 2, 3 or 4 insertions, deletions, substitutions or a combination thereof, in certain aspects 0, 1, 2 or 3 insertions, deletions, substitutions or a combination thereof, more in certain aspects 0; 1 or 2 insertions, deletions, substitutions or a combination thereof, and in certain aspects 0 or 1 insertion, deletion, substitution or a combination thereof with respect to the VH chain depicted in FIG. 1, 2 or 3. The one or more amino acid insertions, deletions, substitutions or a combination thereof are in certain aspects not in the CDR1, CDR2 and/or CDR3 region of the VH chain. They are also in certain aspects not present in the FR4 region. An amino acid substitution is in certain aspects a conservative amino acid substitution.

Rational methods have evolved toward minimizing the content of non-human residues in the human context. Various methods are available to successfully graft the antigen-binding property of an antibody onto another antibody. The binding properties of antibodies may rest predominantly in the exact sequence of the CDR3 region, often supported by the sequence of the CDR1 and CDR2 regions in the variable domain combined with the appropriate structure of the variable domain as a whole.

CDR sequences can be defined using different methods, including, but not limited to, according to the Kabat numbering scheme (Kabat et al., J. Biol. Chem. 252:6609-6616 (1977); and/or Kabat et al., U.S. Dept. of Health and Human Services, “Sequences of proteins of immunological interest” (1991)), the Chothia numbering scheme (Chothia et al., J. Mol. Biol. 196:901-917 (1987); Chothia et al., Nature 342:877-883, 1989; and/or Al-Lazikani B. et al., J. Mol. Biol., 273:927-948 (1997)), the numbering system of Honegger and Plukthun (Honegger and Pluckthun, J. Mol. Biol., 309:657-670 (2001)), the numbering system of MacCallum (MacCallum et al., J. Mol. Biol. 262:732-745 (1996); and/or Abhinandan and Martin, Mol. Immunol., 45:3832-3839 (2008)), the numbering system of Lefranc (Lefranc M. P. et al., Dev. Comp. Immunol., 27:55-77 (2003); and/or Honegger and Pluckthun, J. Mol. Biol., 309:657-670 (2001)), or according to IMGT (discussed in Giudicelli et al., Nucleic Acids Res. 25:206-21 1 (1997)).

Each of these numbering schemes base their definition of CDRs on a predicted contribution of amino acid residues in the heavy or light chain variable region to antigen binding. Hence, each method to identify CDRs can be used to identify the CDRs of the binding domains of the present disclosure. In certain embodiments, the heavy chain CDRs of a binding domain of the present disclosure is according to Kabat, Chothia, or IMGT. In certain aspects, the heavy chain CDRs of a binding domain of the present disclosure is according to Kabat. In certain embodiments, the heavy chain CDRs of a binding domain of the present disclosure is according to Chothia. In certain embodiments, the heavy chain CDRs of a binding domain of the present disclosure is according to IMGT. In certain embodiments, the light chain CDRs of a binding domain of the present disclosure is according to Kabat. In certain embodiments, the light chain CDRs of a binding domain of the present disclosure is according to Chothia. In certain aspects, the light chain CDRs of a binding domain of the present disclosure is according to IMGT. The amino acid sequence of a heavy chain CDR region as depicted herein is determined with the Kabat definition.

Various methods are presently available to graft CDR regions onto a suitable variable domain of another antibody. Some of these methods are reviewed in J. C. Almagro1 and J. Fransson (2008) Frontiers in Bioscience 13, 1619-1633, which is included by reference herein. The disclosure therefore further provides a humanized or in certain aspects human bispecific antibody as included in the treatment of the present disclosure comprising a first antigen-binding site that binds EGFR and a second antigen-binding site that binds cMET, wherein the variable domain comprising the EGFR binding site comprises a VH CDR3 sequence as depicted for MF3370 in FIG. 1, and wherein the variable domain comprising the cMET binding site comprises a VH CDR3 region as depicted for MF4356 in FIG. 1. The VH variable region comprising the EGFR binding site in certain aspects comprises the sequence of the CDR1 region, CDR2 region and the CDR3 region of a VH chain as depicted for MF3370 in FIG. 1. The VH variable region comprising the cMET binding site in certain aspects comprises the sequence of the CDR1 region, CDR2 region and the CDR3 region of a VH chain as depicted for MF4356 in FIG. 1. CDR grafting may also be used to produce a VH chain with the CDR regions of a VH of FIG. 1, but having a different framework. The different framework may be of another human VH, or of a different mammal. The disclosure therefore further provides a humanized or in certain aspects human bispecific antibody as included in the treatment of the present disclosure comprising a first antigen-binding site that binds EGFR and a second antigen-binding site that binds cMET, wherein the variable domain comprising the EGFR binding site comprises a VH CDR3 sequence as depicted for MF8233 in FIG. 2, and wherein the variable domain comprising the cMET binding site comprises a VH CDR3 region as depicted for MF8230 in FIG. 3. The VH variable region comprising the EGFR binding site in certain aspects comprises the sequence of the CDR1 region, CDR2 region and the CDR3 region of a VH chain as depicted for MF8233 in FIG. 2. The VH variable region comprising the cMET binding site in certain aspects comprises the sequence of the CDR1 region, CDR2 region and the CDR3 region of a VH chain as depicted for MF8230 in FIG. 3. CDR grafting may also be used to produce a VH chain with the CDR regions of a VH of FIG. 2 or FIG. 3, but having a different framework. The different framework may be of another human VH, or of a different mammal.

The present disclosure therefore further provides a human or humanized bispecific antibody as included in the treatment of the present disclosure comprising a first antigen-binding site that binds EGFR and a second antigen-binding site that binds cMET, wherein the variable domain comprising the EGFR binding site comprises a VH CDR3 sequence as depicted for MF3370 in FIG. 1, and wherein the variable domain comprising the cMET binding site comprises a VH CDR3 region as depicted for MF8230 in FIG. 3. The VH variable region comprising the EGFR binding site in certain aspects comprises the sequence of the CDR1 region, CDR2 region and the CDR3 region of a VH chain as depicted for MF3370 in FIG. 1. The VH variable region comprising the cMET binding site in certain aspects comprises the sequence of the CDR1 region, CDR2 region and the CDR3 region of a VH chain as depicted for MF8230 in FIG. 3. CDR grafting may also be used to produce a VH chain with the CDR regions of a VH of FIG. 2 or FIG. 3, but having a different framework. The different framework may be of another human VH, or of a different mammal.

The present disclosure therefore further provides a human or humanized bispecific antibody as included in the treatment of the present disclosure comprising a first antigen-binding site that binds EGFR and a second antigen-binding site that binds cMET, wherein the variable domain comprising the EGFR binding site comprises a VH CDR3 sequence as depicted for MF8233 in FIG. 2, and wherein the variable domain comprising the cMET binding site comprises a VH CDR3 region as depicted for MF4356 in FIG. 3. The VH variable region comprising the EGFR binding site in certain aspects comprises the sequence of the CDR1 region, CDR2 region and the CDR3 region of a VH chain as depicted for MF8233 in FIG. 2. The VH variable region comprising the cMET binding site in certain aspects comprises the sequence of the CDR1 region, CDR2 region and the CDR3 region of a VH chain as depicted for MF4356 in FIG. 3. CDR grafting may also be used to produce a VH chain with the CDR regions of a VH of FIG. 2 or FIG. 3, but having a different framework. The different framework may be of another human VH, or of a different mammal.

The present disclosure therefore further provides a humanized or in certain aspects human bispecific antibody as included in the treatment of the present disclosure comprising a first antigen-binding site that binds EGFR and a second antigen-binding site that binds cMET, wherein the variable domain comprising the EGFR binding site comprises a VH CDR3 sequence as depicted for MF8233 in FIG. 2, and wherein the variable domain comprising the cMET binding site comprises a VH CDR3 region as depicted for MF8230 in FIG. 3. The VH variable region comprising the EGFR binding site in certain aspects comprises the sequence of the CDR1 region, CDR2 region and the CDR3 region of a VH chain as depicted for MF8233 in FIG. 2. The VH variable region comprising the cMET binding site in certain aspects comprises the sequence of the CDR1 region, CDR2 region and the CDR3 region of a VH chain as depicted for MF8230 in FIG. 3. CDR grafting may also be used to produce a VH chain with the CDR regions of a VH of FIG. 2 or FIG. 3, but having a different framework. The different framework may be of another human VH, or of a different mammal.

Methods for generating sequence variants are well known in the art. One can take a random approach in generating sequence variants or a targeted approach, where one can for instance aim at introducing variations that are likely to increase or decrease binding affinity. Routine methods for affinity maturing antibody binding domains are widely known in the art, see for instance Tabasinezhad M. et al. Immunol Lett. 2019; 212:106-113. One can also aim at introducing variations that mitigate developability risks with a view on producing a binding domain, or moiety comprising such binding domain, at large scale. Variations may be introduced that are likely not to cause a loss in binding specificity and/or affect binding affinity. Whether amino acid residues within the CDRs and/or framework regions can be substituted, for instance with a conservative amino acid residue, and without, or substantially without, loss in binding specificity and/or affinity, can be determined by methods well known in the art. Experimental examples include, but are not limited to, for instance, alanine scanning (Cunningham B C, Wells J A. Science. 1989; 244 (4908): 1081-5), and deep mutational scanning (Araya C L, Fowler D M. Trends Biotechnol. 2011; 29 (9): 435-42). Computational methods have also been developed that can predict the effect of amino acid variation, such as for instance described in Sruthi C K, Prakash M. PLOS One. 2020; 15 (1): e0227621, Choi Y. et al. PLOS One. 2012; 7 (10): e46688, and Munro D, Singh M. Bioinformatics. 2020; 36 (22-23): 5322-9.

Further provided herein are any variant anti-human EGFR and c-MET binding domains produced by the above described method; binding moieties, such as antibodies, comprising any of said variant binding domains; a pharmaceutical composition comprising any of said variant anti-human EGFR and c-MET binding domains or binding moieties; nucleic acids encoding any of said variant binding domains; vectors and cells comprising said nucleic acids; and use of said variant binding domains or pharmaceutical composition for the treatment of cancer.

The present disclosure therefore further provides a human or humanized bispecific antibody as included in the treatment of the present disclosure comprising a first antigen-binding site that binds EGFR and a second antigen-binding site that binds cMET, wherein the variable domain comprising the EGFR binding site comprises a VH CDR3 sequence as depicted for MF8232 in FIG. 2, and wherein the variable domain comprising the cMET binding site comprises a VH CDR3 region as depicted for MF8230 in FIG. 3. The VH variable region comprising the EGFR binding site in certain aspects comprises the sequence of the CDR1 region, CDR2 region and the CDR3 region of a VH chain as depicted for MF8232 in FIG. 2. The VH variable region comprising the cMET binding site in certain aspects comprises the sequence of the CDR1 region, CDR2 region and the CDR3 region of a VH chain as depicted for MF8230 in FIG. 3. CDR grafting may also be used to produce a VH chain with the CDR regions of a VH of FIG. 2 or FIG. 3, but having a different framework. The different framework may be of another human VH, or of a different mammal.

The present disclosure therefore further provides a human or humanized bispecific antibody as included in the treatment of the present disclosure comprising a first antigen-binding site that binds EGFR and a second antigen-binding site that binds cMET, wherein the variable domain comprising the EGFR binding site comprises a VH CDR3 sequence as depicted for MF8232 in FIG. 2, and wherein the variable domain comprising the cMET binding site comprises a VH CDR3 region as depicted for MF4356 in FIG. 3. The VH variable region comprising the EGFR binding site in certain aspects comprises the sequence of the CDR1 region, CDR2 region and the CDR3 region of a VH chain as depicted for MF8232 in FIG. 2. The VH variable region comprising the cMET binding site in certain aspects comprises the sequence of the CDR1 region, CDR2 region and the CDR3 region of a VH chain as depicted for MF4356 in FIG. 3. CDR grafting may also be used to produce a VH chain with the CDR regions of a VH of FIG. 2 or FIG. 3, but having a different framework. The different framework may be of another human VH, or of a different mammal.

The present disclosure therefore further provides a humanized or in certain aspects human bispecific antibody as included in the treatment of the present disclosure comprising a first antigen-binding site that binds EGFR and a second antigen-binding site that binds cMET, wherein the variable domain comprising the EGFR binding site comprises a VH CDR3 sequence as depicted for MF8232 in FIG. 2, and wherein the variable domain comprising the cMET binding site comprises a VH CDR3 region as depicted for MF8230 in FIG. 3. The VH variable region comprising the EGFR binding site in certain aspects comprises the sequence of the CDR1 region, CDR2 region and the CDR3 region of a VH chain as depicted for MF8232 in FIG. 2. The VH variable region comprising the cMET binding site in certain aspects comprises the sequence of the CDR1 region, CDR2 region and the CDR3 region of a VH chain as depicted for MF8230 in FIG. 3. CDR grafting may also be used to produce a VH chain with the CDR regions of a VH of FIG. 2 or FIG. 3, but having a different framework. The different framework may be of another human VH, or of a different mammal.

The disclosure further provides a human or humanized bispecific antibody as included in the treatment of the present disclosure comprising a first variable domain that binds EGFR and a second variable domain that binds cMET wherein the first variable domain comprises a heavy chain variable region with the amino acid sequence of MF3370 as depicted in FIG. 2 having at most 10, in certain aspects 0, 1, 2, 3, 4, 5, 6, 7, 8, 9 or 10 and in certain aspects having 0, 1, 2, 3, 4 or 5 amino acid insertions, deletions, substitutions or a combination thereof and wherein the second variable domain comprises a heavy chain variable region that comprises the amino acid sequence of MF4356 depicted in FIG. 3 (SEQ ID NO: 23) with 0-10 in certain aspects 0-5 amino acid insertions, deletions, substitutions, additions or a combination thereof. The disclosure further provides a human or humanized bispecific antibody as included in the treatment of the present disclosure comprising a first variable domain that binds EGFR and a second variable domain that binds cMET wherein the first variable domain comprises a heavy chain variable region with the amino acid sequence of MF8233 as depicted in FIG. 2 having at most 10, in certain aspects 0, 1, 2, 3, 4, 5, 6, 7, 8, 9 or 10 and in certain aspects having 0, 1, 2, 3, 4 or 5 amino acid insertions, deletions, substitutions or a combination thereof and wherein the second variable domain comprises a heavy chain variable region that comprises the amino acid sequence of MF8230 depicted in FIG. 3 (SEQ ID NO: 13) with 0-10 in certain aspects 0-5 amino acid insertions, deletions, substitutions, additions or a combination thereof.

The disclosure further provides a human or humanized bispecific antibody as included in the treatment of the present disclosure comprising a first variable domain that binds EGFR and a second variable domain that binds cMET wherein the first variable domain comprises a heavy chain variable region with the amino acid sequence of MF3370 as depicted in FIG. 2 having at most 10, in certain aspects 0, 1, 2, 3, 4, 5, 6, 7, 8, 9 or 10 and in certain aspects having 0, 1, 2, 3, 4 or 5 amino acid insertions, deletions, substitutions or a combination thereof and wherein the second variable domain comprises a heavy chain variable region that comprises the amino acid sequence of MF8230 depicted in FIG. 3 (SEQ ID NO: 13) with 0-10 in certain aspects 0-5 amino acid insertions, deletions, substitutions, additions or a combination thereof.

The disclosure further provides a human or humanized bispecific antibody as included in the treatment of the present disclosure comprising a first variable domain that binds EGFR and a second variable domain that binds cMET wherein the first variable domain comprises a heavy chain variable region with the amino acid sequence of MF8233 as depicted in FIG. 2 having at most 10, in certain aspects 0, 1, 2, 3, 4, 5, 6, 7, 8, 9 or 10 and in certain aspects having 0, 1, 2, 3, 4 or 5 amino acid insertions, deletions, substitutions or a combination thereof and wherein the second variable domain comprises a heavy chain variable region that comprises the amino acid sequence of MF4356 depicted in FIG. 3 (SEQ ID NO: 23) with 0-10 in certain aspects 0-5 amino acid insertions, deletions, substitutions, additions or a combination thereof.

The disclosure further provides a human or humanized bispecific antibody as included in the treatment of the present disclosure comprising a first variable domain that binds EGFR and a second variable domain that binds cMET wherein the first variable domain comprises a heavy chain variable region with the amino acid sequence of MF8232 as depicted in FIG. 2 having at most 10, in certain aspects 0, 1, 2, 3, 4, 5, 6, 7, 8, 9 or 10 and in certain aspects having 0, 1, 2, 3, 4 or 5 amino acid insertions, deletions, substitutions or a combination thereof and wherein the second variable domain comprises a heavy chain variable region that comprises the amino acid sequence of MF4356 depicted in FIG. 3 (SEQ ID NO: 23) with 0-10 in certain aspects 0-5 amino acid insertions, deletions, substitutions, additions or a combination thereof.

The disclosure further provides a human or humanized bispecific antibody as included in the treatment of the present disclosure comprising a first variable domain that binds EGFR and a second variable domain that binds cMET wherein the first variable domain comprises a heavy chain variable region with the amino acid sequence of MF8232 as depicted in FIG. 2 having at most 10, in certain aspects 0, 1, 2, 3, 4, 5, 6, 7, 8, 9 or 10 and in certain aspects having 0, 1, 2, 3, 4 or 5 amino acid insertions, deletions, substitutions or a combination thereof and wherein the second variable domain comprises a heavy chain variable region that comprises the amino acid sequence of MF8230 depicted in FIG. 3 (SEQ ID NO: 13) with 0-10 in certain aspects 0-5 amino acid insertions, deletions, substitutions, additions or a combination thereof.

The mentioned at most 15, in certain aspects 0, 1, 2, 3, 4, 5, 6, 7, 8, 9 or 10 and in certain aspects 0, 1, 2, 3, 4 or 5 amino acid substitutions are in certain aspects conservative amino acid substitutions, the insertions, deletions, substitutions or a combination thereof are in certain aspects not in the CDR3 region of the VH chain, in certain aspects not in the CDR1, CDR2 or CDR3 region of the VH chain and in certain aspects not in the FR4 region.

Various methods are available to produce bispecific antibodies. One method involves the expression of two different heavy chains and two different light chains in a cell and collecting antibody that is produced by the cell. Antibody produced in this way will typically contain a collection of antibodies with different combinations of heavy and light chains, some of which are the desired bispecific antibody. The bispecific antibody can subsequently be purified from the collection. The ratio of bispecific to other antibodies that are produced by the cell can be increased in various ways. In certain aspects, the ratio is increased by expressing not two different light chains but a common light chain in the cell. When a common light chain is expressed with the two different heavy chains, the ratio of bispecific antibody to other antibody that is produced by the cell is significantly improved over the expression of two different light chains. The ratio of bispecific antibody that is produced by the cell can be further improved by stimulating the pairing of two different heavy chains with each other over the pairing of two identical heavy chains. Methods and means are disclosed for producing bispecific antibodies (from a single cell), whereby means are provided that favor the formation of bispecific antibodies over the formation of monospecific antibodies. These methods can also be favorably employed in the present disclosure. Thus the disclosure in certain aspects provides a method for producing a bispecific antibody from a single cell, wherein said bispecific antibody comprises two CH3 domains that are capable of forming an interface, said method comprising providing in said cell a) a first nucleic acid molecule encoding a 1st CH3 domain comprising heavy chain, b) a second nucleic acid molecule encoding a 2nd CH3 domain comprising heavy chain, wherein said nucleic acid molecules are provided with means for preferential pairing of said 1st and 2nd CH3 domain comprising heavy chains, said method further comprising the step of culturing said host cell and allowing for expression of said two nucleic acid molecules and harvesting said bispecific antibody from the culture. Said first and second nucleic acid molecules may be part of the same nucleic acid molecule, vector or gene delivery vehicle and may be integrated at the same site of the host cell's genome. Alternatively, said first and second nucleic acid molecules are separately provided to said cell.

Certain aspects provides a method for producing a bispecific antibody according to the disclosure from a single cell, wherein said bispecific antibody comprises two CH3 domains that are capable of forming an interface, said method comprising providing:
a cell having a) a first nucleic acid molecule encoding a heavy chain comprising an antigen binding site that binds EGFR and that contains a 1st CH3 domain, and b) a second nucleic acid molecule encoding a heavy chain comprising an antigen-binding site that binds ErbB-3 and that contains a 2nd CH3 domain, wherein said nucleic acid molecules are provided with means for preferential pairing of said 1st and 2nd CH3 domains,
said method further comprising the step of culturing said cell and allowing for expression of the proteins encoded by said two nucleic acid molecules and harvesting said bispecific IgG antibody from the culture. In certain aspects, said cell also has a third nucleic acid molecule encoding a common light chain. Said first, second and third nucleic acid molecule may be part of the same nucleic acid molecule, vector or gene delivery vehicle and may be integrated at the same site of the host cell's genome. Alternatively, said first, second and third nucleic acid molecules are separately provided to said cell. In certain aspects, the common light chain is based on O12, in certain aspects it is the rearranged germline human kappa light chain IgVκ1 39*01/IGJK1*01, as described above. Means for preferential pairing of said 1st and said 2nd CH3 domain are in certain aspects the corresponding mutations in the CH3 domain of the heavy chain coding regions. The preferred mutations to preferentially produce bispecific antibodies are the amino acid substitutions L351K and T366K (EU-numbering) in the first CH3 domain and the amino acid substitutions L351D and L368E in the second CH3 domain, or vice versa. Further provided is therefore a method according to the disclosure for producing a bispecific antibody, wherein said first CH3 domain comprises the amino acid substitutions L351K and T366K (EU-numbering) and wherein said second CH3 domain comprises the amino acid substitutions L351D and L368E, said method further comprising the step of culturing said cell and allowing for expression of proteins encoded by said nucleic acid molecules and harvesting said bispecific antibody from the culture. Also provided is a method according to the disclosure for producing a bispecific antibody, wherein said first CH3 domain comprises the amino acid substitutions L351D and L368E (EU-numbering) and wherein said second CH3 domain comprises the amino acid substitutions L351K and T366K, said method further comprising the step of culturing said cell and allowing for expression of said nucleic acid molecules and harvesting said bispecific antibody from the culture. Antibodies that can be produced by these methods are also part of the present disclosure. The CH3 hetero-dimerization domains are in certain aspects IgG1 hetero-dimerization domains. The heavy chain constant regions comprising the CH3 hetero-dimerization domains are in certain aspects IgG1 constant regions.

In one embodiment of the disclosure includes a nucleic acid molecule encoding an antibody heavy chain variable region. The nucleic acid molecule (typically an in vitro, isolated or recombinant nucleic acid molecule) in certain aspects encodes a heavy chain variable region as depicted in FIG. 2 or FIG. 3, or a heavy chain variable region as depicted in FIG. 2 or FIG. 3 having 1, 2, 3, 4 or 5 amino acid insertions, deletions, substitutions or combination thereof. In certain aspects the nucleic acid molecule comprises codon optimized nucleic acid sequence coding for an amino acid sequence as depicted in FIG. 2 or FIG. 3. The codon optimization is optimized for the species and/or the cell type of the antibody producing cell. For example, for CHO production the nucleic acid sequence of the molecule is codon optimized for Chinese hamster cells. The disclosure further provides a nucleic acid molecule encoding a heavy chain of FIG. 2 or FIG. 3.

A nucleic acid molecule as used in the present disclosure is typically but not exclusively a ribonucleic acid (RNA) or a deoxyribonucleic acid (DNA). Alternative nucleic acids are available for a person skilled in the art. A nucleic acid according to the disclosure is for instance comprised in a cell. When said nucleic acid is expressed in said cell, said cell can produce an antibody according to the disclosure. Therefore, in one embodiment of the disclosure includes a cell comprising an antibody according to the disclosure and/or a nucleic acid according to the disclosure. Said cell is in certain aspects an animal cell, in certain aspects a mammal cell, in certain aspects a primate cell, in certain aspects a human cell. A suitable cell is any cell capable of comprising and in certain aspects producing an antibody according to the disclosure and/or a nucleic acid according to the disclosure.

The disclosure further provides a cell comprising an antibody according to the disclosure. In certain aspects said cell (typically an in vitro, isolated or recombinant cell) produces said antibody. Said cell can also be a stored cell that is able to produce said antibody when taken out of storage and cultured. In certain aspects said cell is a hybridoma cell, a Chinese hamster ovary (CHO) cell, an NS0 cell or a PER-C6™ cell. In certain aspects, said cell is a CHO cell. Further provided is a cell culture comprising a cell according to the disclosure. Various institutions and companies have developed cell lines for the large scale production of antibodies, for instance for clinical use. Non-limiting examples of such cell lines are CHO cells, NS0 cells or PER.C6™ cells. These cells are also used for other purposes such as the production of proteins. Cell lines developed for industrial scale production of proteins and antibodies are herein further referred to as industrial cell lines. Thus certain aspects includes use of a cell line developed for the large scale production of antibody for the production of an antibody of the disclosure, including in certain aspects a cell for producing an antibody comprising a nucleic acid molecule that codes for a VH, a VL, and/or a heavy chain as depicted in FIG. 2 or FIG. 3.

The disclosure further provides a method for producing an antibody comprising culturing a cell of the disclosure and harvesting said antibody from said culture. In certain aspects said cell is cultured in a serum free medium. In certain aspects said cell is adapted for suspension growth. Further provided is an antibody obtainable by a method for producing an antibody according to the disclosure. The antibody is in certain aspects purified from the medium of the culture. In certain aspects said antibody is affinity purified.

A cell of the present disclosure is for instance a hybridoma cell line, a CHO cell, a 293F cell, an NS0 cell or another cell type known for its suitability for antibody production for clinical purposes. In certain aspects, said cell is a human cell. In certain aspects a cell that is transformed by an adenovirus E1 region or a functional equivalent thereof. In certain aspects, such a cell line is the PER.C6™ cell line or equivalent thereof. In certain aspects, said cell is a CHO cell or a variant thereof. In certain aspects a variant that makes use of a Glutamine synthetase (GS) vector system for expression of an antibody.

Antibodies of the present disclosure can be produced at levels >50 mg/L after transient transfection in suspension 293F cells. The bispecific antibodies can be purified to greater than 98% purity with yields >70%. Analytical characterization studies show bispecific lgG1 antibody profiles that are comparable to bivalent monospecific lgG1. In terms of functional activity a bispecific antibody of the disclosure can demonstrate superior potency compared to cetuximab in vitro and in vivo.

The disclosure further provides a pharmaceutical composition comprising an antibody according to the present disclosure. The pharmaceutical composition in certain aspects comprises a in certain aspects pharmaceutically acceptable excipient or carrier.

An antibody can comprise a label, in certain aspects a label for in vivo imaging. Such a label is typically not necessary for therapeutic applications. In for instance a diagnostic setting, a label can be helpful. For instance in visualizing target cells in the body. Various labels are suited and many are well known in the art. In certain aspects the label is a radioactive label for detection. In certain aspects, the label is an infrared label. In certain aspects the infrared label is suited for in vivo imaging. Various infrared labels are available to the person skilled in the art. Preferred infrared labels are for instance, IRDye 800; IRDye 680RD; IRDye 680LT; IRDye 750; IRDye 700DX; IRDye 800RS IRDye 650; IRDye 700 phosphoramidite; IRDye 800 phosphoramidite (LI-COR USA; 4647 Superior Street; Lincoln, Nebraska).

The disclosure further provides a method for the treatment of a subject that has a tumor or is at risk of having said tumor comprising administering to the subject in need thereof an antibody or a pharmaceutical composition according to the disclosure. The tumor is in certain aspects an EGFR, cMET or EGFR/cMET positive tumor. Before start of said treatment, the method in certain aspects further comprises determining whether said subject has such an EGFR, cMET or EGFR/cMET positive tumor. The disclosure further provides an antibody or pharmaceutical composition of the disclosure for use in the treatment of a subject that has or is at risk of having an EGFR, cMET or EGFR/cMET positive tumor.

To establish whether a tumor is positive for EGFR the skilled person can for instance determine the EGFR amplification and/or immuno-histochemistry staining. At least 10% of the tumor cells in a biopsy should be positive. The biopsy can also contain 20%, 30% 40% 50% 60% 70% or more positive cells. To establish whether a tumor is positive for cMET the skilled person can for instance determine the cMET amplification and/or staining in immunohistochemistry. At least 10% of the tumor cells in a biopsy should be positive. The biopsy can also contain 20%, 30% 40% 50% 60% 70% or more positive cells.

The cancer or tumor may be an EGFR, cMET or EGFR/cMET positive cancer. In one preferable aspect, the disclosure provides treatment of the EGFR, cMET or EGFR/cMET positive cancer that is lung cancer, in particular non-small cell lung cancer, head and neck cancer, in particular head and neck squamous cell carcinoma; gastric cancer, in particular gastric adenocarcinoma; esophageal cancer, in particular esophageal squamous cell carcinoma; gastric/esophageal junction cancer, breast cancer, colon cancer, pancreatic cancer, ovarian cancer, colorectal cancer or bladder cancer. The disclosure can be applied to a wide range of EGFR, cMET or EGFR/cMET positive cancers, like lung cancer, in certain aspects including non-small cell lung cancer. The subject is in certain aspects a human subject. The subject is in certain aspects a subject eligible for antibody therapy using an EGFR specific antibody such as cetuximab. In certain aspects the disclosure may in certain aspects treat a subject that comprises a tumor, in certain aspects an EGFR/cMET positive cancer, in certain aspects a tumor/cancer with an EGFR RTK resistant phenotype, an EGFR monoclonal antibody resistant phenotype or a combination thereof.

As used herein, the term ‘cancer’ applies to the same as the term ‘tumor’, such that treatment of a tumor also applies to treatment of cancer.

The amount of antibody to be administered to a patient is typically in the therapeutic window, meaning that a sufficient quantity is used for obtaining a therapeutic effect, while the amount does not exceed a threshold value leading to an unacceptable extent of side-effects. The lower the amount of antibody needed for obtaining a desired therapeutic effect, the larger the therapeutic window will typically be. An antibody according to the disclosure exerting sufficient therapeutic effects at low dosage is, therefore, preferred. The dosage can be in range of the dosing regimen of cetuximab. The dosage can also be lower. In certain aspects, the dose is 1000 mg, 1500 mg, or 2000 mg. Dosing may be once every week or once every two weeks.

In certain aspects, the cancer is lung cancer, in particular non-small cell lung cancer; head and neck cancer, in particular head and neck squamous cell carcinoma; gastric cancer, in particular gastric adenocarcinoma; esophageal cancer, in particular esophageal squamous cell carcinoma; gastric/esophageal junction cancer, breast cancer, colon cancer, pancreatic cancer, ovarian cancer, colorectal cancer or bladder cancer and the antibody of the present disclosure is administered in an amount of 1000 mg, typically a flat dose of 1000 mg once every week.

In certain aspects, the cancer is lung cancer, in particular non-small cell lung cancer; head and neck cancer, in particular head and neck squamous cell carcinoma; gastric cancer, in particular gastric adenocarcinoma; esophageal cancer, in particular esophageal squamous cell carcinoma; gastric/esophageal junction cancer, breast cancer, colon cancer, pancreatic cancer, ovarian cancer, colorectal cancer or bladder cancer and the antibody of the present disclosure is administered in an amount of 1000 mg, typically a flat dose of 1000 mg once every two weeks.

In certain aspects, the cancer is lung cancer, in particular non-small cell lung cancer; head and neck cancer, in particular head and neck squamous cell carcinoma; gastric cancer, in particular gastric adenocarcinoma; esophageal cancer, in particular esophageal squamous cell carcinoma; gastric/esophageal junction cancer, breast cancer, colon cancer, pancreatic cancer, ovarian cancer, colorectal cancer or bladder cancer and the antibody of the present disclosure is administered in an amount of 1500 mg, typically a flat dose of 1500 mg once every two weeks.

In certain aspects, the cancer is lung cancer, in particular non-small cell lung cancer; head and neck cancer, in particular head and neck squamous cell carcinoma; gastric cancer, in particular gastric adenocarcinoma; esophageal cancer, in particular esophageal squamous cell carcinoma; gastric/esophageal junction cancer, breast cancer, colon cancer, pancreatic cancer, ovarian cancer, colorectal cancer or bladder cancer and the antibody of the present disclosure is administered in an amount of 2000 mg, typically a flat dose of 2000 mg once every two weeks.

In certain aspects, the bispecific antibody binding EGFR and cMET is provided to a subject at a dosage of 1000, 1500 or 2000 mg, in particular using a flat dose regimen. A flat dose regimen offers several advantages over body-surface or weight dosing as it reduces preparation time and reduces potential dose calculation mistakes. In certain aspects, the bispecific antibody is administered once every week (Q1W), once every 2 weeks (Q2W) or once every 3 weeks (Q3W). In certain aspects, the bispecific antibody is administered once every two weeks. In the art, such a dosing scheme is noted as Q2W. In certain embodiments, the flat dose regimen disclosed herein is suitable for use in adults and/or in subjects weighing at least 35 kg. As is understood by the skilled person, the dosage can be administered over time. As is understood by the skilled person, the term “flat dose” or “flat dose regimen” means that the subject undergoes a dosing regimen wherein on each day the subject is scheduled to receive the bispecific antibody with substantially the same predetermined amount thereof which amount is irrespective of the subjects' body weight. According to certain aspects, the subject is provided a flat, weekly dose of 1000 mg bispecific antibody. Alternatively, the subject is provided a flat, biweekly dose of 1000 mg bispecific antibody. Alternatively, the subject is provided a flat, biweekly dose of 1500 mg bispecific antibody. Alternatively, the subject is provided a flat, biweekly dose of 2000 mg bispecific antibody.

A bispecific antibody according to the present disclosure in certain aspects induces less skin toxicity as compared to cetuximab under otherwise similar conditions. A bispecific antibody according to the disclosure in certain aspects produces less proinflammatory chemokines, in certain aspects of CXCL14 as compared to cetuximab under otherwise similar conditions. A bispecific antibody according to the disclosure in certain aspects induces less impairment of antimicrobial RNAses, in certain aspects Rnase 7, as compared to cetuximab under otherwise similar conditions.

The present disclosure describes among others antibodies that target the EGFR and cMET receptors and result in potent proliferation inhibition of cancer cell lines in vitro and tumor growth inhibition in vivo. A bispecific antibody of the can combine low toxicity profiles with high efficacy. An antibody of the disclosure can be useful in various types and lines of EGFR-targeted therapies. An antibody of the disclosure can have an increased therapeutic window when compared to an antibody that binds the same antigen(s) with both arms. A bispecific antibody of the disclosure can exhibit better growth inhibitory effects in vitro, in vivo or a combination thereof when compared to the cetuximab antibody.

The disclosure provides a bispecific antibody for use in the treatment of subject that may have one or more of a variety of different kinds of tumors. The tumor may be an EGFR positive tumor, a cMET positive tumor or an EGFR and cMET positive tumor. The tumor may be resistant to treatment with an EGFR or cMET tyrosine kinase inhibitor. In certain aspects, the EGFR tyrosine kinase inhibitor is a third generation EGFR tyrosine kinase inhibitor, in certain aspects Osimertinib or an analogue thereof. In certain aspects, the cMET tyrosine inhibitor is or comprises capmatinib or tepotinib. In this and other embodiments the tumor may be an HGF-associated tumor.

An EGFR-positive tumor is typically a tumor that has an EGFR activating mutation. An EGFR activating mutation is a mutation of EGFR that results in activation of the EGF/EGFR signaling pathway. The EGFR activating mutation may be important for a cancerous state of the tumor. One of the ways in which such tumors can become insensitive to EGFR targeted therapy is by activation of the HGF/cMET signaling pathway. The tumor may be an HGF-associated tumor. Activation of the cMET/HGF signaling pathway is one of the ways in which an EGFR-positive tumor can escape treatment with an EGFR-targeted therapy. The cMET/HGF pathway can be activated in various ways. Various methods of activation are described in the art some of which are detailed herein. An antibody of the disclosure is particularly suited for the treatment of tumors wherein activation of the cMET/HGF signaling pathway is associated with the presence of or excess of HGF. Such cMET positive tumors are referred to as HGF-associated tumors or HGF-dependent tumors. An antibody of the disclosure can also be used to at least in part inhibit this possible escape mechanism of EGFR positive tumors. Such tumors can escape EGFR-targeted therapy through the selected outgrowth of tumor cells wherein, in addition, the cMET/HGF signaling pathway is activated. Such cells may be present at the start of the EGFR-targeted therapy. Such cells have a selective growth advantage over HGF/cMET signaling negative tumor cells. The tumor may be a tumor wherein the HGF/cMET signaling pathway is activated. The tumor may be a tumor that is associated with elevated levels of hepatocyte growth factor (HGF) or overexpression of the HGF receptor c-Met. The tumor may be a tumor wherein growth is driven by the EGF and/or HGF. A tumor is said to be driven by a certain growth factor if the signaling pathway is activated in cells of the tumor in response to the presence of the growth factor and removal of the growth factor results in inhibition of the growth of the cells of the tumor. Reduction can be measured by reduced cell division and/or induced cell kill such as apoptosis. A tumor is an HGF-associated tumor if under conditions that would otherwise be permissive for the growth of the tumor, the tumor growths or growths faster in the presence of HGF.

EGFR-targeted therapies for various tumors have been reviewed by Vecchione et al., EGFR-targeted therapy.” Experimental cell research Vol 317 (2011): 2765-2771. In general EGFR-targeted therapy is a therapy with a molecule that interacts with EGFR and inhibits EGFR-mediated signaling in the cell.

The method of treatment or antibody for use in the treatment as indicated herein in certain aspects further comprises the step of determining whether the tumor is an HGF-associated tumor. In certain aspects, an antibody of the disclosure can inhibit growth of an HGF-associated tumor.

Where herein ranges are given as between number 1 and number 2, the range includes the number 1 and number 2. For instance a range of between 2-5 includes the number 2 and 5.

When herein reference is made to an affinity that is higher than another, the Kd=lower than the other Kd. For the avoidance of doubt a Kd of 10e-9 M is lower than a Kd of 10e-8 M. The affinity of an antibody with a Kd of 10e-9 M for a target is higher than when the Kd is 10e-8 M.

In certain aspects, at the start of treatment, at least one, more than one or all of the following inclusion factors IF1-IF7 are applicable to subjects for treatment. In certain aspects, the subject comprises or complies with all of inclusion factors IF1-IF8:
IF1. Having an age of equal to or higher than 18 years at signature of informed consent,
IF2. Having a histologically or cytologically confirmed solid tumor with evidence of metastatic or locally advanced unresected disease that is incurable.
IF3.1. For subjects who have failed prior standard first-line treatment: Subjects have progressed on or are intolerant to therapies that are known to provide clinical benefit. Subjects have either NSCLC harboring activating EGFR mutations including tyrosine kinase inhibitor (TKI) sensitizing mutations and/or approved TKI-resistance mutations, or any activating c-MET mutation/amplification.
IF3.2. For subjects with prior first or higher anti-cancer treatment: Having progressed on or be intolerant to therapies that are known to provide clinical benefit. Patients must have: either NSCLC harboring an EGFR exon 20 insertion, that has progressed after platinum doublets, or NSCLC harboring cMet exon 14 skipping mutation in second (or higher) line patients, capmatinib and tepotinib resistant population, or treated with a cMET TKI or cMET TKI naive, or selected solid tumors (including GC/GEJ, HNSCC, e.g. HNSCC refractory to approved treatment (regardless of driving mutations), or ESCC) harboring an EGFR or cMet driving mutation or cMET alteration, NSCLC patients with rare EGFR mutations (G719X, L861Q, and S768I) or subjects with NSCLC harboring a c-MET exon 14 skipping mutation and treatment naïve for advanced disease. Subjects are exhausted or intolerant to all approved therapies with proven clinical benefit or have no available, approved, therapeutic options.
IF4. Having availability of FFPE embedded after progression at the latest therapy, archival or a fresh tumor tissue sample.
IF5. Having measurable disease as defined by RECIST version 1.1 by radiologic methods (patients with non-measurable but evaluable disease can be included in the dose escalation part).
IF6. Having an Eastern Cooperative Oncology Group (ECOG) performance status of 0 or 1.
IF7. Having a life expectancy ≥12 weeks.
IF8 Having adequate organ function, as established by at least one or all of:
IF8.1 Absolute neutrophil count (ANC) ≥1.5×10⁹/L.
IF8.2 Hemoglobin >9 g/dL.
IF8.3 Platelets ≥100×10⁹/L.
IF8.4 Corrected total serum calcium within normal ranges.
IF8.5 Serum magnesium within normal ranges (or corrected with supplements).
IF8.6 Serum potassium within normal ranges.
IF8.7 Alanine aminotransferase (ALT) and aspartate aminotransferase (AST) equal to or less than 3.0× the upper limit of normal (ULN) and total bilirubin equal to or less than 1.5×ULN, with the proviso that in cases of a liver involvement or malignancy, ALT/AST is equal to or less than 5×ULN and total bilirubin is equal to or less than 2×ULN.
IF8.8 For patients with Gilbert's syndrome, conjugated bilirubin has a value within normal limits.
IF8.9 Serum creatinine equal to or less than 1.5×ULN or creatinine clearance equal to or higher than 50 mL/min calculated according to the Cockroft and Gault formula or the MDRD formula for patients aged over 65 years.
IF8.10 Serum albumin >3.3 g/dL

In certain aspects, all values for organ function measurements according to IF8 have an upper limit observed with healthy subjects.

In certain aspects, the subject for treatment comprises one or more factors selected from the group consisting of IF1-IF8. In certain aspects, the subject for treatment comprises factors IF2, IF3, IF5 and IF8. In certain aspects, the subject for treatment comprises all of the factors IF1-IF8.

In certain aspects, at the start of treatment, at least one, more than one or all of the following exclusion factors EF1-EF16 are applicable to subjects for treatment:
EF1. Having central nervous system metastases that:
EF1.1: are untreated and symptomatic, whereas subjects having asymptomatic lesions can be included if considered stable;
EF1.2 require radiation or surgery;
EF1.3 require continued steroid therapy (>10 mg prednisone or equivalent) to control symptoms within 14 days of administration prior to administration of the first dose. Subjects with other central nervous system metastasis are allowed.
EF2. Having known leptomeningeal involvement.
EF3. Participation in another clinical study or treatment with any investigational drug within 4 weeks prior to administration of the first dose.
EF4. Administration of systemic anticancer therapy or immunotherapy within 4 weeks or 5 half-lives, whichever is shorter, of the first dose of study drug. For cytotoxic agents that have major delayed toxicity (e.g., mitomycin C, nitrosoureas), a washout period of 6 weeks is required.
EF5. Having undergone major surgery or radiotherapy within 3 weeks of administration of the first dose. Subjects who received prior radiotherapy to equal or more than 25% of bone marrow at any time are not eligible.
EF6. Having persistent grade of more than 1 clinically significant toxicity, related to prior antineoplastic therapies (except for alopecia); with the proviso that stable sensory neuropathy with a grade equal to 2 or lower of NCI-CTCAE v5.0 and hypothyroidism with a grade equal to 2 or lower which is stable on hormone replacement is not excluded.
EF7. Having a history of hypersensitivity reaction or any toxicity attributed to human proteins or any of the excipients that warranted permanent cessation of these agents.
EF8. Having a history of clinically significant cardiovascular disease including, but not limited to:
EF8.1 Diagnosis of deep vein thrombosis or pulmonary embolism within 1 month prior to first dose of study drug, or any of the following within 6 months prior to the first dose of study drug: myocardial infarction, unstable angina, stroke, transient ischemic attack, coronary/peripheral artery bypass graft, or any acute coronary syndrome.
EF8.2 Prolonged QT interval >480 msec or clinically significant cardiac arrythmia or electrophysiologic disease (i.e., placement of implantable cardioverter defibrillator or atrial fibrillation with uncontrolled rate). Patients with cardiac pacemakers who are clinically stable are eligible.
EF8.3 Uncontrolled (persistent) arterial hypertension: systolic blood pressure >180 mm Hg and/or diastolic blood pressure >100 mm Hg.
EF8.4 Congestive heart failure (CHF) defined as New York Heart Association (NYHA) class III-IV or hospitalization for CHF within 6 months of the first dose of study drug.
EF8.5 Clinically significant pericardial effusion.
EF8.6 Myocarditis.
EF9. Having a history of interstitial lung disease including drug-induced interstitial lung disease, radiation pneumonitis that requires treatment with prolonged steroids or other immune suppressive agents within 1 year.
EF10. Having a previous or concurrent malignancy, excluding non-basal cell carcinomas of skin or carcinoma in situ of the uterine cervix, unless the tumor was treated with curative or palliative intent and the previous or concurrent malignancy condition does not affect the assessment of safety and efficacy of the study drug.
EF11. Having current serious illness or medical conditions including, but not limited to uncontrolled active infection, clinically significant pulmonary, metabolic or psychiatric disorders.
EF12. Having active Hepatitis B infection (HBsAg positive) without receiving antiviral treatment. Subjects with active hepatitis B (HbsAg positive) must receive antiviral treatment with lamivudine, tenofovir, entecavir, or other antiviral agents, starting at least 7 days or more before administration of the first dose. Subjects with antecedents of Hepatitis B (anti-HBc positive, HbsAg and HBV-DNA negative) are eligible.
EF13. Having a positive test for Hepatitis C ribonucleic acid (HCV RNA); Subjects in whom HCV infection resolved spontaneously (positive HCV antibodies without detectable HCV-RNA) or those who achieved a sustained virological response after antiviral treatment and show absence of detectable HCV RNA equal to or more than 6 months (with the use of IFN-free regimens) or equal to or more than 12 months (with the use of IFN-based regimens) after cessation of antiviral treatment are eligible.
EF14. Having a known history of HIV (HIV 1/2 antibodies). Patients with HIV with undetectable viral load are allowed. HIV testing is not required unless mandated by local health authority or regulations.
EF15. In case of sexually active male and female patients of childbearing potential agree to use one of the following methods of birth control during the entire duration of the study and for 6 months after final administration of PB19478:
   combined (estrogen and progestogen containing) hormonal contraception associated with inhibition of ovulation (oral, intravaginal, transdermal)
   progestogen-only hormonal contraception associated with inhibition of ovulation (oral, injectable, implantable)
   intrauterine device (IUD)
   intrauterine hormone-releasing system (IUS)
   bilateral tubal occlusion
   vasectomized partner
   sexual abstinence
EF16. Being pregnant or breast-feeding.

In certain aspects, the subject for treatment complies with one or more factors selected from the group consisting of EF1-EF16. In certain aspects, the subject for treatment complies with all of the factors EF1-EF16.

A reference herein to a patent document or other matter which is cited is not to be taken as an admission that that document or matter was known or that the information it contains was part of the common general knowledge as at the priority date of any of the claims.

The conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

For the purpose of clarity and a concise description, features are described herein as part of the same or separate embodiments, however, it will be appreciated that the scope of the disclosure may include embodiments having combinations of all or some of the features described.

### BRIEF DESCRIPTION OF THE DRAWINGS

FIG. 1. Amino acid sequence of the heavy chain variable regions of variable domains referred to in this application.

FIG. 2. MF3370 and variants thereof. The CDR1, CDR2 and CDR3 sequences in MF8226 are underlined from left to right. The CDRs in the other sequences are at the corresponding positions (according to Kabat).

FIG. 3. MF4356 and variants thereof. The CDR1, CDR2 and CDR3 sequences in MF4356 are underlined from left to right. The CDRs in the other sequences are at the corresponding positions (according to Kabat).

FIG. 4. Common light chain used in mono- and bispecific IgG.

FIG. 4A: Common light chain amino acid sequence. FIG. 4B: Common light chain variable domain DNA sequence and translation (IGKV1-39/jk1). FIG. 4C: Common light chain constant region DNA sequence and translation. FIG. 4D: IGKV1-39/jk5 common light chain variable domain translation. FIG. 4E: V-region IGKV1-39A.

FIG. 5. IgG heavy chains for the generation of bispecific molecules. FIG. 5A: CH1 region. FIG. 5B: hinge region. FIG. 5C: CH2 region. FIG. 5D: CH2 containing L235G and G236R silencing substitutions. FIG. 5E: CH3 domain containing substitutions L351K and T366K (KK). FIG. 5F; CH3 domain containing substitutions L351D and L368E (DE).

FIG. 6. Results of Western blot analysis of model LXFE2478 showing EGFR, cMET and HGF protein levels.

FIG. 7. Overview of the treatment schedule. Antibody or vehicle was administered as monotherapy over a period of 5 weeks.

FIG. 8. Effects of antibody therapy on tumor volume in NSCLC PDX model LXFE2478 harboring a mutation in exon 20. Tumor volume growth curve representing groups 1-5 shown at different time points (mean TV+SEM).

FIG. 9. Overview of the treatment schedule. Antibody or vehicle was administered as monotherapy over a period of 3 weeks.

FIG. 10. Effects of antibody therapy on tumor volume in NSCLC CDX model HCC827/ER1 harboring a mutation in exon 19. Tumor volume growth curve representing vehicle and antibody groups shown at different time points (mean TV+SEM).

FIG. 11. Animal survival curves of mice carrying NSCLC CDX model HCC827/ER1 harboring a mutation in exon 19 vs vehicle.

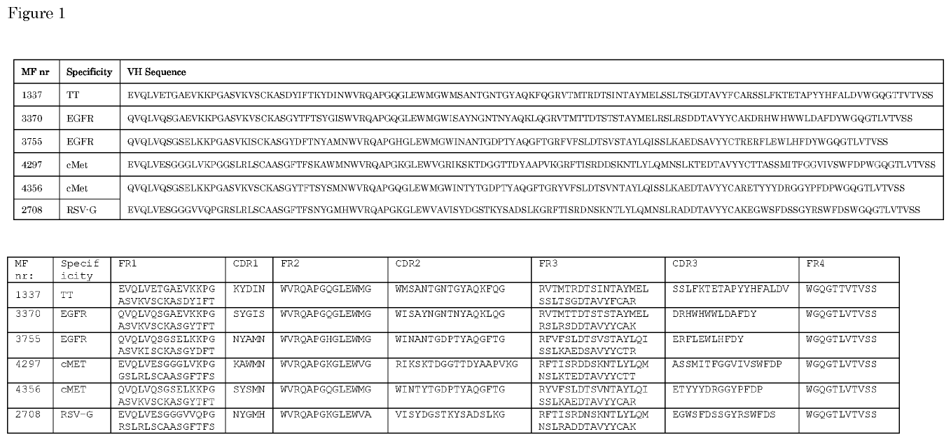

### CLAUSES

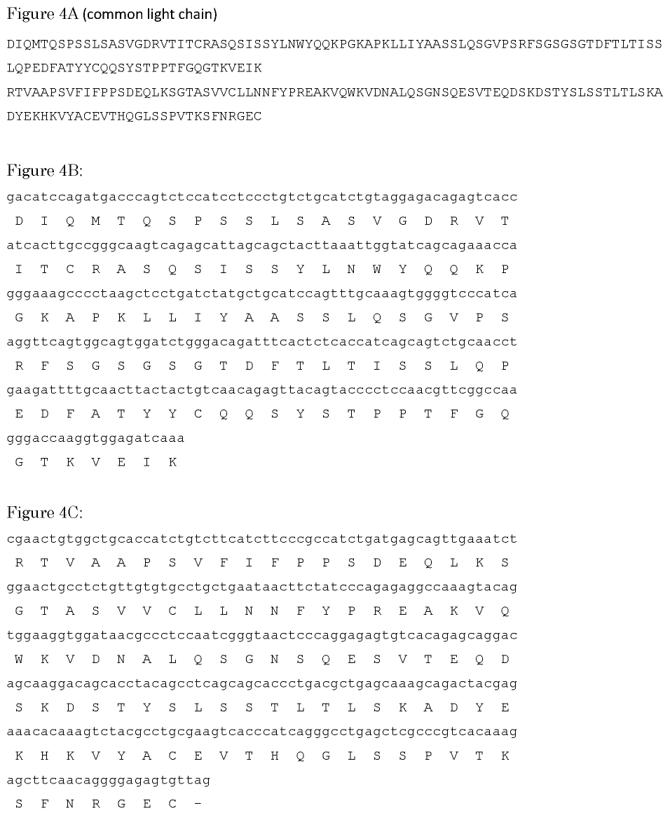

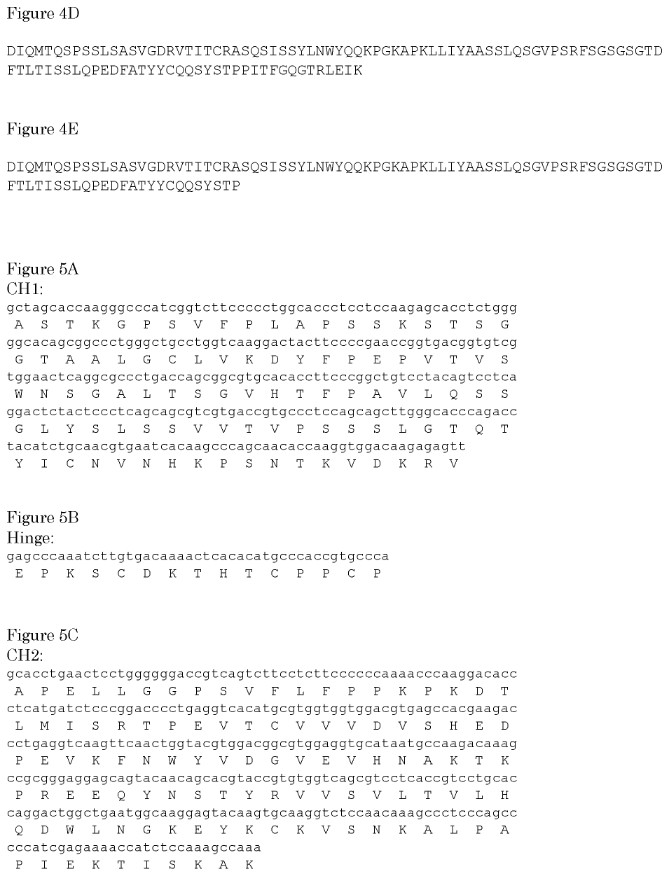

1. A bispecific antibody that comprises a first variable domain that can bind an extracellular part of human epidermal growth factor receptor (EGFR) and a second variable domain that can bind an extracellular part of human MET Proto-Oncogene, Receptor Tyrosine Kinase (cMET), wherein the first variable domain comprises a heavy chain variable region with a CDR1 sequence SYGIS; a CDR2 sequence WISAYX₁X₂NTNYAQKLQG and a CDR3 comprising the sequence X₃X₄X₅X₆HWWLX₇A, wherein X₁=N or S; X₂=A or G; X₃=D or G; X₄=R, S or Y; X₅=H, L or Y; X₆=D or W and X₇=D or G; with 0-5 amino acid insertions, deletions, substitutions, additions or a combination thereof at a position other than X₁-X₇ and wherein the second variable domain comprises a heavy chain variable region with the amino acid sequence of one of the sequences of SEQ ID NO: 1-23 with 0-10 in certain aspects 0-5 amino acid insertions, deletions, substitutions, additions or a combination thereof, for use in a method of treatment of a cancer in a subject which has received prior treatment with i) a third-generation EGFR tyrosine kinase inhibitor; or ii) a chemotherapy and a tyrosine kinase inhibitor; iii) a cMET tyrosine kinase inhibitor or iv) treatment of a cancer in a subject which has not received any prior anticancer treatment.
2. Method of treating a cancer in a subject which has received prior treatment with i) a third-generation EGFR tyrosine kinase inhibitor or ii) a chemotherapy and an EGFR tyrosine kinase inhibitor, iii) a cMET tyrosine kinase inhibitor or iv) treatment of a cancer in a subject which has not received any prior anticancer treatment, said treatment comprising administering to the subject an effective amount of a bispecific antibody that comprises a first variable domain that can bind an extracellular part of human epidermal growth factor receptor (EGFR) and a second variable domain that can bind an extracellular part of human MET Proto-Oncogene, Receptor Tyrosine Kinase (cMET), wherein the first variable domain comprises a heavy chain variable region with a CDR1 sequence SYGIS; a CDR2 sequence WISAYX₁X₂NTNYAQKLQG and a CDR3 comprising the sequence X₃X₄X₅X₆HWWLX₇A, wherein X₁=N or S; X₂=A or G; X₃=D or G; X₄=R, S or Y; X₅=H, L or Y; X₆=D or W and X₇=D or G; with 0-5 amino acid insertions, deletions, substitutions, additions or a combination thereof at a position other than X₁-X₇ and wherein the second variable domain comprises a heavy chain variable region with the amino acid sequence of one of the sequences of SEQ ID NO: 1-23 with 0-10 in certain aspects 0-5 amino acid insertions, deletions, substitutions, additions or a combination thereof.
3. Use of a bispecific antibody that comprises a first variable domain that can bind an extracellular part of human epidermal growth factor receptor (EGFR) and a second variable domain that can bind (or binds) an extracellular part of human MET Proto-Oncogene, Receptor Tyrosine Kinase (cMET) wherein the first variable domain comprises a heavy chain variable region with a CDR1 sequence SYGIS; a CDR2 sequence WISAYX₁X₂NTNYAQKLQG and a CDR3 comprising the sequence X₃X₄X₅X₆HWWLX₇A, wherein X₁=N or S; X₂=A or G; X₃=D or G; X₄=R, S or Y; X₅=H, L or Y; X₆=D or W and X₇=D or G; with 0-5 amino acid insertions, deletions, substitutions, additions or a combination thereof at a position other than X₁-X₇ and wherein the second variable domain comprises a heavy chain variable region with the amino acid sequence of one of the sequences of SEQ ID NO: 1-23 with 0-10 in certain aspects 0-5 amino acid insertions, deletions, substitutions, additions or a combination thereof, in the manufacture of a medicament for the treatment of a cancer in a subject which has received prior treatment with i) a third-generation EGFR tyrosine kinase inhibitor or ii) a chemotherapy and an EGFR tyrosine kinase inhibitor, iii) a cMET tyrosine kinase inhibitor or iv) treatment of a cancer in a subject which has not received any prior anticancer treatment.
4. The use or method according to any one of the preceding clauses, wherein said subject or cancer which has received prior treatment according to i) is resistant to treatment with a third-generation EGFR tyrosine kinase inhibitor.
5. The use or method according to any one of clauses 1-3, wherein said subject or cancer which has received prior treatment according to ii) is resistant to treatment with a first-, second and/or third-generation tyrosine kinase inhibitor.
6. The use or method according to any one of clauses 1-3, wherein said subject or cancer which has received prior treatment according to iii) is resistant to treatment with a cMET tyrosine kinase inhibitor.
7. The use or method according to any one of the preceding clauses, wherein administration of said bispecific antibody according to i) comprises a second line treatment, and administration according to ii) or iii) comprises a third line treatment.
8. The use or method according to any one of the preceding clauses, wherein said first-generation EGFR tyrosine kinase inhibitor comprises or is gefitinib, erlotinib, gefitinib or icotinib.
9. The use or method according to any one of the preceding clauses, wherein said second-generation EGFR tyrosine kinase inhibitor comprises or is afatinib, dacomitinib, XL647, AP26113, CO-1686 or neratinib.
10. The use or method according to any one of the preceding clauses, wherein said third-generation EGFR tyrosine kinase comprises Osimertinib, Lazertinib, Alflutinib, Rezivertinib, Rociletinib, Olmutinib, Almonertinib, Abivertinib, ASK120067, Befotertinib, Olmutinib, Rociletinib or SH-1028 (nazartinib (EGF816), naquotinib (ASP8273), mavelertinib (PF-0647775), Olafertinib (CK-101), Keynatinib, or ES-072, preferably Osimertinib.
11. The use or method according to any one of the preceding clauses, wherein said cMET tyrosine kinase inhibitor comprises or is capmatinib, tepotinib, crizotenib, cabozantinib, savolitinib, Glesatinib, Sitravatinib, BMS-777607, Merestinib, Tivantinib, Golvatinib, Foretinib, AMG-337 or BMS-794833, preferably capmatinib or tepotinib.
12. The use or method according to any one of the preceding clauses, wherein said chemotherapy comprises platinum-based chemotherapy, cisplatin, carboplatin, oxaliplatin, paclitaxel, docetaxel, gemcitabine, vinorelbine, etoposide or pemetrexed, or any combination thereof, preferably a combination comprising cisplatin or carboplatin.
13. The use or method according to any one of the preceding clauses, wherein said cancer is an EGFR positive cancer, a cMET positive cancer or an EGFR and cMET positive cancer.
14. The use or method according to any one of the preceding clauses, wherein said cancer comprises an EGFR aberration, a cMET aberration or an EGRF and cMET aberration.
15. The use or method according to any one of the preceding clauses, wherein said cancer comprises an activating EGFR mutation, an approved tyrosine kinase inhibitor resistance mutation, a tertiary tyrosine kinase inhibitor resistance mutation (such as L718X (e.g. L718Q), G719X (e.g. G719A), L792X (e.g. L792H), G796X (e.g. G796R, G796S, G796D), C797X, C797X (e.g. C797S, C797G), a mutation that reduces binding of a third generation tyrosine kinase inhibitor to EGFR (e.g. L792X, L718X), an acquired tyrosine kinase inhibitor resistance mutation (such as C797X, L792X, G796X, G724X, S768X, L718X or an exon 20 insertion mutation), an EGFR gene amplification, a cMET mutation or cMET aberration.
16. The use or method according to any one of the preceding clauses, wherein said cancer comprises an exon 19 deletion mutation, preferably an in-frame exon 19 deletion, an exon 20 missense mutation (e.g. T790M) or an exon 21 mutation, such as L858R.
17. The use or method according to any one of the preceding clauses, wherein said cancer comprises an EGFR exon 20 mutation, preferably an exon 20 insertion mutation.
18. The use or method according to any one of the preceding clauses, wherein said cancer comprises an acquired tyrosine kinase inhibitor resistance mutation such as a mutation which confers resistance to Osimertinib, including G724X (e.g. G724S), S768X (e.g. S768I), T790X (e.g. T790M), L792X (e.g. L792H), C797X (including C797S and C797G), L798X (e.g. L798I).
19. The use or method according to any one of the preceding clauses, wherein said cancer comprises an exon 20 (762-823) mutation selected from a near-loop insertion (positions 767-772), a far-loop insertion (positions 773-775), preferably V769_D770insASV, D770_N771insSVD, H773_V774insNPH, H773_V774insH, D770_N771insG, D770delinsGY, N771_P772insN, V774_C775insHV, D770_N771insGL, H773_V774insPH, A763_Y764insFQEA, D770_N771delinsEGN, D770_N771insGD, D770_N771insH, D770_N771insP, H773_V774insAH, H773_V774insGNPH, H773delinsSNPY, N771_P772insH, N771_P772insVDN, N771delinsGY, N771delinsKH, N771delinsRD, P772_H773delinsHNPY, P772_H773insGT, P772_H773insPNP, P772_H773insT, V769_D770insA, V769_D770insGG, V769_D770insGSV, V769_D770insGVV and V769_D770insMASV; or mutations T790M, L792X (e.g. L792H, C796X (e.g. G796R, G796S, G796D), C797X (e.g. C797S, C797G), L798I, or an in-frame exon 20 insertion, such as M766_A767insASV or H773-V774insNPH, Ins761 (EAFQ), Ins770 (ASV), Ins771 (G), Ins774 (NPH), M766_A7671 ns A, S768_V769InsSVA, P772_H773InsNS, D761_E762InsX1-7, A763_Y764InsX1-7, Y764_Y765 InsX1-7, M766_A767InsX1-7, A767_V768 InsX1-7, S768_V769 InsX1-7) V769_D770 InsX1-7) D770_N771 InsX1-7) N771_P772 InsX1-7) P772_H773 InsX1-7, H773_V774 InsX1-7, or V774_C775 InsX1-7.
20. The use or method according to any one of the preceding clauses, wherein said cancer comprises a cMET aberration, such as a cMET amplification, cMET overexpression, increased signaling of the cMET pathway, a cMET gene amplification, increased cMET protein activity and/or increased HGF expression.
21. The use or method according to any one of the preceding clauses, wherein said cancer comprises a cMET exon14 skipping mutation.
22. The use or method according to any one of the preceding clauses, wherein said cancer is non-small cell lung cancer (NSCLC), head and neck cancer, in particular head and neck squamous cell carcinoma; gastric cancer, in particular gastric adenocarcinoma; esophageal cancer, in particular esophageal squamous cell carcinoma; gastric/esophageal junction cancer, breast cancer, colon cancer, pancreatic cancer, ovarian cancer, colorectal cancer or bladder cancer.
23. The use or method according to any one of the preceding clauses, wherein said cancer is NSCLC comprising an activating EGFR mutation, EGFR tyrosine kinase inhibitor sensitizing mutation (such as an exon 19 deletion and L858X), an acquired EGFR tyrosine kinase inhibitor resistance mutation, (such as T790X, C797X, L792X, L798X) an approved EGFR tyrosine kinase inhibitor resistance mutation, an EGFR exon 20 insertion mutation, an activating c-MET mutation, preferably an exon 14 skipping mutation, or a cMET amplification, preferably comprising MET/CEP7>5 or cfDNA≥2 copies, increased HGF expression or any combination thereof.
24. The use or method according to any one of the preceding clauses, wherein said cancer is advanced or metastatic cancer.
25. The use or method according to any one of the preceding clauses, wherein said cancer or patient has progressed after having received said prior therapy for advanced or metastatic cancer.
26. The use or method according to any one of the preceding clauses, wherein treatment comprises a diagnostic step for assessing whether said cancer is an EGFR positive and/or cMET positive cancer or assessing the presence of an EGFR and/or cMET aberration in said cancer.
27. The use or method according to any one of the preceding clauses, wherein treatment comprises a diagnostic step of determining EGFR amplification, immunohistochemistry staining, determining the amount of one or more biomarkers in tumor tissue, blood or serum, such as the amount of EGFR/c-MET signaling pathway activity, measuring soluble EGFR and soluble c-MET in blood or serum and/or measuring EGFR and c-MET target expression in tumor cells.
28. The use or method according to any one of the preceding clauses, wherein the subject is a human subject.
29. The use or method according to any one of the preceding clauses, wherein the antibody is a human antibody.
30. The use or method according to any one of the preceding clauses, wherein the antibody is ADCC enhanced.
31. The use or method according to any one of the preceding clauses, wherein the antibody is an IgG1 format antibody having an anti-EGFR, anti-cMET stoichiometry of 1:1.
32. The use or method according to any one of the preceding clauses, wherein the antibody has one variable domain that can bind EGFR and one variable domain that can bind cMET.
33. The use or method according to any one of the preceding clauses, wherein the variable domain that can bind human EGFR can also bind cynomolgus and mouse EGFR.
34. The use or method according to any one of the preceding clauses, wherein the variable domain that can bind human EGFR binds to domain III of human EGFR.
35. The use or method according to any one of the preceding clauses, wherein the variable domain that can bind cMET blocks the binding of antibody 5D5 to cMET.
36. The use or method according to any one of the preceding clauses, wherein the variable domain that can bind cMET blocks the binding of HGF to cMET.
37. The use or method according to any one of the preceding clauses, wherein the amino acids at positions 405 and 409 in one CH3 domain are the same as the amino acids at the corresponding positions in the other CH3 domain (EU-numbering).
38. The use or method according to any one of the preceding clauses, wherein
X₁=N; X₂=G; X₃=D; X₄═S; X₅=Y; X₆=W and X₇=G;
X₁=N; X₂=A; X₃=D; X₄═S; X₅=Y; X₆=W and X₇=G;
X₁═S; X₂=G; X₃=D; X₄═S; X₅=Y; X₆=W and X₇=G;
X₁=N; X₂=G; X₃=D; X₄=R; X₅=H; X₆=W and X₇=D;
X₁=N; X₂=A; X₃=D; X₄=R; X₅=H; X₆=W and X₇=D;
X₁═S; X₂=G; X₃=D; X₄=R; X₅=H; X₆=W and X₇=D;
X₁=N; X₂=G; X₃=G; X₄=Y; X₅=L; X₆=D and X₇=G;
X₁=N; X₂=A; X₃=G; X₄=Y; X₅=L; X₆=D and X₇=G; or
X₁═S; X₂=G; X₃=G; X₄=Y; X₅=L; X₆=D and X₇=G
39. The use or method according to any one of the preceding clauses, wherein X₁=N; X₂=G; X₃=D; X₄=R; X₅=H; X₆=W and X₇=D; or X₁=N; X₂=A; X₃=D; X₄=R; X₅=H; X₆=W and X₇=D; or X₁═S; X₂=G; X₃=D; X₄=R; X₅=H; X₆=W and X₇=D.
40. The use or method according to any one of the preceding clauses, wherein X₁=N; X₂=G; X₃=D; X₄=R; X₅=H; X₆=W and X₇=D; or X₁=N; X₂=A; X₃=D; X₄=R; X₅=H; X₆=W and X₇=D.
41. The use or method according to any one of the preceding clauses, wherein the heavy chain variable region of the second variable domain comprises the amino acid sequence of one of the sequences of SEQ ID NO: 1-3; 7; 8; 10; 13; 15; 16; 17; 21; 22 or 23 with 0-10 preferably 0-5 amino acid insertions, deletions, substitutions, additions or a combination thereof.
42. The use or method according to any one of the preceding clauses, wherein the heavy chain variable region of the second variable domain comprises the amino acid sequence of one of the sequences of SEQ ID NO: 2; 7; 8; 10; 13 or 23 with 0-10 preferably 0-5 amino acid insertions, deletions, substitutions, additions or a combination thereof.
43. The use or method according to any one of the preceding clauses, wherein the first variable domain comprises a heavy chain variable region with a CDR1 sequence SYGIS; a CDR2 sequence WISAYNGNTNYAQKLQG and a CDR3 comprising the sequence DRHWHWWLDA and wherein the second variable domain comprises a heavy chain variable region with a CDR1 sequence SYSMN; a CDR2 sequence WINTYTGDPTYAQGFTG and a CDR3 sequence ETYYYDRGGYPFDP.
44. The use or method according to any one of the preceding clauses, wherein the first variable domain comprises a heavy chain variable region with a CDR1 sequence SYGIS; a CDR2 sequence WISAYNANTNYAQKLQG and a CDR3 comprising the sequence DRHWHWWLDA and wherein the second variable domain comprises a heavy chain variable region with a CDR1 sequence TYSMN; a CDR2 sequence WINTYTGDPTYAQGFTG and a CDR3 comprising the sequence ETYFYDRGGYPFDP.
45. The use or method according to any one of the preceding clauses, wherein the first and second variable domain comprise a common light chain, preferably a light chain of FIG. 4B.
46. The use or method according to any one of the preceding clauses, wherein the first and second variable domain comprise a light chain with a CDR1, CDR2 and CDR3 amino acid sequence of QSISSY, AAS, and QQSYSTP, respectively (according to IMGT).
47. The use or method according to any one of the preceding clauses, which antibody inhibits HGF induced growth of an HGF-growth responsive cell.
48. The use or method according to any one of the preceding clauses, which antibody inhibits EGF induced growth of an EGF-growth responsive cell.
49. The use or method according to any one of the preceding clauses, wherein the subject is administered with 1000 mg bispecific antibody, in particular using a flat dose of 1000 mg.
50. The use or method according to clause 49, wherein the bispecific antibody is administered once every week.
51. The use or method according to clause 49, wherein the bispecific antibody is administered once every two weeks.
52. The use or method according to any one of clauses 1-48, wherein the subject is administered with 1500 mg bispecific antibody, in particular using a flat dose of 1500 mg.
53. The use or method according to clause 52, wherein the bispecific antibody is administered once every two weeks.
54. The use or method according to any one of clauses 1-48, wherein the subject is administered with 2000 mg bispecific antibody, in particular using a flat dose of 2000 mg.
55. The use or method according to clause 54, wherein the bispecific antibody is administered once every two weeks.
56. The use or method according to any one of the preceding clauses, wherein the subject under iv) has not received any prior anticancer treatment with a cMET inhibitor.
57. The use or method according to any one of the preceding clauses, wherein the subject under iv) has not received any prior anticancer treatment with a cMET TKI inhibitor.
58. The use or method according to any one of the preceding clauses, wherein the subject under iv) has not received any prior anticancer treatment with capmatinib.
59. The use or method according to any one of the preceding clauses, wherein the subject under iv) has not received any prior anticancer treatment with tepotinib.
60. The use or method according to any one of the preceding clauses, wherein the subject under iv) has not received any prior anticancer treatment with savolitinib.

### EXAMPLES

As used herein “MFXXXX” wherein X is independently a numeral 0-9, refers to a Fab comprising a variable domain wherein the VH has the amino acid sequence identified by the 4 digits. Unless otherwise indicated the light chain variable region of the variable domain typically has a sequence of FIG. 4A, typically 4b. “MFXXXX VH” refers to the amino acid sequence of the VH identified by the 4 digits. The MF further comprises a constant region of a light chain and a constant region of a heavy chain that normally interacts with a constant region of a light chain. PG refers to a monospecific antibody comprising identical heavy and light chains. PB refers to a bispecific antibody with two different heavy chains. The VH variable regions of the heavy chains differ and typically also the CH3 region, wherein one of the heavy chains has a KK mutation of its CH3 domain and the other has the complementing DE mutation of its CH3 domain (see for reference PCT/NL2013/050294 (published as WO2013/157954).

Reference is made to PCTNL/2018/050537 (published as WO2019/031965) for details on production of antibodies of the present disclosure. Bispecific antibodies binding EGFR and cMET suitable for use in the appended examples and for use in the methods of the invention include those of Tables 3, 4, 5 and 6. In particular, bispecific antibody PB19478 is suitably used in the appended examples.

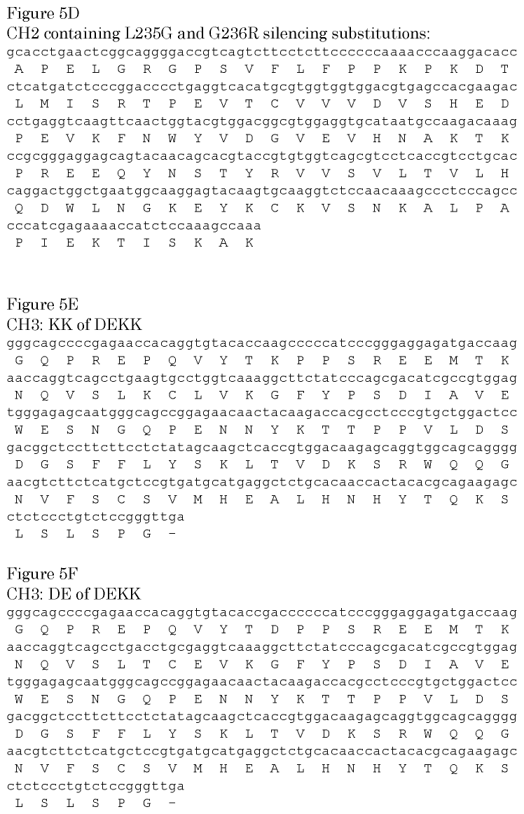

Each bispecific antibody comprises two VH as specified by the MF numbers capable of binding EGFR and cMET respectively, further comprises an Fc tail with a KK/DE CH3 heterodimerization domain as indicated in FIG. 5e and FIG. 5f, respectively, a CH2 domain as indicated by FIG. 5d, a hinge domain as indicated by FIG. 5b, a CH1 domain as indicated by FIG. 5a and a common light chain as indicated by FIGS. 4a-e. For example, a bispecific antibody indicated by MF8233×MF8230 has the above general sequences and a variable domain with a VH with the sequence of MF8233 and a variable domain with a VH with the sequence of MF8230 and is in certain aspects used in the appended examples.

### Example 1: Materials and Methods

### Cell Lines:

EBC-1 [JCRB0820], PC-9 [RCB0446], H358 [ATCCR CRL-5807™], HCC827 [ATCC® CRL-2868™], MKN-45 [DSMZ ACC 409] N87 [ATCCRCRL-5822™ ] and A431 [ATCCR CRL-1555™] cell lines were purchased and routinely maintained in growth media supplemented with 10% heat inactivated fetal bovine serum (FBS). HEK293F Freestyle cells were obtained from Invitrogen and routinely maintained in 293 FreeStyle medium.

cDNA Constructs:
Generation of cMET and EGFR Expression Vectors for Generation of Stable Cell Lines (cMET and EGFR) and for Immunization (cMET)

Full length cDNA of each target including unique restriction sites for cloning and kozak consensus sequence for efficient translation was either synthetized, or obtained via PCR amplification on a commercially available expression construct, containing the target cDNA, with specific primers that introduced unique restriction sites for cloning and kozak consensus sequence for efficient translation. The full length cDNA of each target was cloned into a eukaryotic expression construct such as pcDNA3.1, whereas the extracellular domains were cloned into pVAX1 and pDisplay. The insert sequences were verified by comparison with NCBI Reference amino acid sequences.

### Amino Acid Sequence Full Length Human EGFR Insert for Expression on the Cell Surface (Identical to GenBank: NP_00533):

MRPSGTAGAALLALLAALCPASRALEEKKVCQGTSNKLTQLGTFEDHFLSLQRMEN
NCEVVLGNLEITYVQRNYDLSFLKTIQEVAGYVLIALNTVERIPLENLQIIRGNMYYE
NSYALAVLSNYDANKTGLKELPMRNLQEILHGAVRFSNNPALCNVESIQWRDIVSSD
FLSNMSMDFQNHLGSCQKCDPSCPNGSCWGAGEENCQKLTKIICAQQCSGRCRGKS
PSDCCHNQCAAGCTGPRESDCLVCRKFRDEATCKDTCPPLMLYNPTTYQMDVNPEG
KYSFGATCVKKCPRNYVVTDHGSCVRACGADSYEMEEDGVRKCKKCEGPCRKVCN
GIGIGEFKDSLSINATNIKHFKNCTSISGDLHILPVAFRGDSFTHTPPLDPQELDILKT
VKEITGFLLIQAWPENRTDLHAFENLEIIRGRTKQHGQFSLAVVSLNITSLGLRSLKEI
SDGDVIISGNKNLCYANTINWKKLFGTSGQKTKIISNRGENSCKATGQVCHALCSPE
GCWGPEPRDCVSCRNVSRGRECVDKCNLLEGEPREFVENSECIQCHPECLPQAMNI
TCTGRGPDNCIQCAHYIDGPHCVKTCPAGVMGENNTLVWKYADAGHVCHLCHPNC
TYGCTGPGLEGCPTNGPKIPSIATGMVGALLLLLVVALGIGLFMRRRHIVRKRTLRRL
LQERELVEPLTPSGEAPNQALLRILKETEFKKIKVLGSGAFGTVYKGLWIPEGEKVKI
PVAIKELREATSPKANKEILDEAYVMASVDNPHVCRLLGICLTSTVQLITQLMPFGCL
LDYVREHKDNIGSQYLLNWCVQIAKGMNYLEDRRLVHRDLAARNVLVKTPQHVKIT
DFGLAKLLGAEEKEYHAEGGKVPIKWMALESILHRIYTHQSDVWSYGVTVWELMTF
GSKPYDGIPASEISSILEKGERLPQPPICTIDVYMIMVKCWMIDADSRPKFRELIIEFSK
MARDPQRYLVIQGDERMHLPSPTDSNFYRALMDEEDMDDVVDADEYLIPQQGFFSS
PSTSRTPLLSSLSATSNNSTVACIDRNGLQSCPIKEDSFLQRYSSDPTGALTEDSIDDT
FLPVPEYINQSVPKRPAGSVQNPVYHNQPLNPAPSRDPHYQDPHSTAVGNPEYLNTV
QPTCVNSTFDSPAHWAQKGSHQISLDNPDYQQDFFPKEAKPNGIFKGSTAENAEYLR
VAPQSSEFIGA

Of which:

-MRPSGTAGAALLALLAALCPASR: signal peptide.
-ALEEKKVCQGTSNKLTQLGTFEDHFLSLQRMENNCEVVLGNLEITYVQRNYD
LSFLKTIQEVAGYVLIALNTVERIPLENLQIIRGNMYYENSYALAVLSNYDANKTGLK
ELPMRNLQEILHGAVRFSNNPALCNVESIQWRDIVSSDFLSNMSMDFQNHLGSCQK
CDPSCPNGSCWGAGEENCQKLTKIICAQQCSGRCRGKSPSDCCHNQCAAGCTGPRE
SDCLVCRKFRDEATCKDTCPPLMLYNPTTYQMDVNPEGKYSFGATCVKKCPRNYVV
TDHGSCVRACGADSYEMEEDGVRKCKKCEGPCRKVCNGIGIGEFKDSLSINATNIKH
FKNCTSISGDLHILPVAFRGDSFTHTPPLDPQELDILKTVKEITGFLLIQAWPENRTDL
HAFENLEIIRGRTKQHGQFSLAVVSLNITSLGLRSLKEISDGDVIISGNKNLCYANTIN
WKKLFGTSGQKTKIISNRGENSCKATGQVCHALCSPEGCWGPEPRDCVSCRNVSRG
RECVDKCNLLEGEPREFVENSECIQCHPECLPQAMNITCTGRGPDNCIQCAHYIDGP
HCVKTCPAGVMGENNTLVWKYADAGHVCHLCHPNCTYGCTGPGLEGCPTNGPKIP
S: ECD of human EGFR.
-IATGMVGALLLLLVVALGIGLFM: predicted TM region.
-RRRHIVRKRTLRRLLQERELVEPLTPSGEAPNQALLRILKETEFKKIKVLGSGA
FGTVYKGLWIPEGEKVKIPVAIKELREATSPKANKEILDEAYVMASVDNPHVCRLLGI
CLTSTVQLITQLMPFGCLLDYVREHKDNIGSQYLLNWCVQIAKGMNYLEDRRLVHR
DLAARNVLVKTPQHVKITDFGLAKLLGAEEKEYHAEGGKVPIKWMALESILHRIYTH
QSDVWSYGVTVWELMTFGSKPYDGIPASEISSILEKGERLPQPPICTIDVYMIMVKCW
MIDADSRPKFRELIIEFSKMARDPQRYLVIQGDERMHLPSPTDSNFYRALMDEEDMD
DVVDADEYLIPQQGFFSSPSTSRTPLLSSLSATSNNSTVACIDRNGLQSCPIKEDSFLQ
RYSSDPTGALTEDSIDDTFLPVPEYINQSVPKRPAGSVQNPVYHNQPLNPAPSRDPHY
QDPHSTAVGNPEYLNTVQPTCVNSTFDSPAHWAQKGSHQISLDNPDYQQDFFPKEA
KPNGIFKGSTAENAEYLRVAPQSSEFIGA: intracellular tail.

Amino acid sequence of extracellular domain of human EGFRvarIII a natural occurring EGFR variant VAR_066493 [Ji H., Zhao X; PNAS 103:7817-7822 (2006)] caused by an in-frame deletion of exons 2-7. The _below indicates the location lacking amino acids 30-297

MRPSGTAGAALLALLAALCPASRALEEKK_GNYVVTDHGSCVRACGADSYEMEEDG
VRKCKKCEGPCRKVCNGIGIGEFKDSLSINATNIKHFKNCTSISGDLHILPVAFRGDS
FTHTPPLDPQELDILKTVKEITGFLLIQAWPENRTDLHAFENLEIIRGRTKQHGQFSL
AVVSLNITSLGLRSLKEISDGDVIISGNKNLCYANTINWKKLFGTSGQKTKIISNRGEN
SCKATGQVCHALCSPEGCWGPEPRDCVSCRNVSRGRECVDKCNLLEGEPREFVENS
ECIQCHPECLPQAMNITCTGRGPDNCIQCAHYIDGPHCVKTCPAGVMGENNTLVWK
YADAGHVCHLCHPNCTYGCTGPGLEGCPTNGPKIPS

Of which:

-MRPSGTAGAALLALLAALCPASR: signal peptide.
-ALEEKK_GNYVVTDHGSCVRACGADSYEMEEDGVRKCKKCEGPCRKVCNGI
GIGEFKDSLSINATNIKHFKNCTSISGDLHILPVAFRGDSFTHTPPLDPQELDILKTVK
EITGFLLIQAWPENRTDLHAFENLEIIRGRTKQHGQFSLAVVSLNITSLGLRSLKEISD
GDVIISGNKNLCYANTINWKKLFGTSGQKTKIISNRGENSCKATGQVCHALCSPEGC
WGPEPRDCVSCRNVSRGRECVDKCNLLEGEPREFVENSECIQCHPECLPQAMNITC
TGRGPDNCIQCAHYIDGPHCVKTCPAGVMGENNTLVWKYADAGHVCHLCHPNCTY
GCTGPGLEGCPTNGPKIPS: ECD of EGFRvarIII

Amino acid sequence chimeric macaque (Macaca mulatta) extra cellular EGFR domain hybrid with human EGFR transmembrane and intracellular domain for expression on the cell surface (Identical to GenBank: XP_014988922.1. Human EGFR sequence underlined in the example below.

MGPSGTAGAALLALLAALCPASRALEEKKVCQGTSNKLTQLGTFEDHFLSLQRMEN
NCEVVLGNLEITYVQRNYDLSFLKTIQEVAGYVLIALNTVERIPLENLQIIRGNMYYE
NSYALAVLSNYDANKTGLKELPMRNLQEILHGAVRFSNNPALCNVESIQWRDIVSSE
FLSNMSMDFQNHLGSCQKCDPSCPNGSCWGAGEENCQKLTKIICAQQCSGRCRGKS
PSDCCHNQCAAGCTGPRESDCLVCRKFRDEATCKDTCPPLMLYNPTTYQMDVNPEG
KYSFGATCVKKCPRNYVVTDHGSCVRACGADSYEMEEDGVRKCKKCEGPCRKVCN
GIGIGEFKDTLSINATNIKHFKNCTSISGDLHILPVAFRGDSFTHTPPLDPQELDILKT
VKEITGFLLIQAWPENRTDLHAFENLEIIRGRTKQHGQFSLAVVSLNITSLGLRSLKEI
SDGDVIISGNKNLCYANTINWKKLFGTSSQKTKIISNRGENSCKATGQVCHALCSPEG
CWGPEPRDCVSCQNVSRGRECVDKCNILEGEPREFVENSECIQCHPECLPQVMNITC
TGRGPDNCIQCAHYIDGPHCVKTCPAGVMGENNTLVWKYADAGHVCHLCHPNCTY
GCTGPGLEGCARNGPKIPSIATGMLGALLLLLVVALGIGLEMRRRHIVRKRTLRRLLQ
ERELVEPLTPSGEAPNQALLRILKETEFKKIKVLGSGAFGTVYKGLWIPEGEKVKIPV
AIKELREATSPKANKEILDEAYVMASVDNPHVCRLLGICLTSTVQLITQLMPFGCLLD
YVREHKDNIGSQYLLNWCVQIAKGMNYLEDRRLVHRDLAARNVLVKTPQHVKITDE
GLAKLLGAEEKEYHAEGGKVPIKWMALESILHRIYTHQSDVWSYGVTVWELMTFGS
KPYDGIPASEISSILEKGERLPQPPICTIDVYMIMVKCWMIDADSRPKFRELIIEFSKM
ARDPQRYLVIQGDERMHLPSPTDSNFYRALMDEEDMDDVVDADEYLIPQQGFFSSPS
TSRTPLLSSLSATSNNSTVACIDRNGLQSCPIKEDSFLQRYSSDPTGALTEDSIDDTEL
PVPEYINQSVPKRPAGSVQNPVYHNQPLNPAPSRDPHYQDPHSTAVGNPEYLNTVQP
TCVNSTFDSPAHWAQKGSHQISLDNPDYQQDFFPKEAKPNGIFKGSTAENAEYLRVA
PQSSEFIGA

Of which:

-MGPSGTAGAALLALLAALCPASR: signal peptide.
-LEEKKVCQGTSNKLTQLGTFEDHFLSLQRMFNNCEVVLGNLEITYVQRNYDL
SFLKTIQEVAGYVLIALNTVERIPLENLQIIRGNMYYENSYALAVLSNYDANKTGLKE
LPMRNLQEILHGAVRFSNNPALCNVESIQWRDIVSSDFLSNMSMDFQNHLGSCQKC
DPSCPNGSCWGAGEENCQKLTKIICAQQCSGRCRGKSPSDCCHNQCAAGCTGPRES
DCLVCRKFRDEATCKDTCPPLMLYNPTTYQMDVNPEGKYSFGATCVKKCPRNYVVT
DHGSCVRACGADSYEMEEDGVRKCKKCEGPCRKVCNGIGIGEFKDSLSINATNIKHF
KNCTSISGDLHILPVAFRGDSFTHTPPLDPQELDILKTVKEITGFLLIQAWPENRTDL
HAFENLEIIRGRTKQHGQFSLAVVSLNITSLGLRSLKEISDGDVIISGNKNLCYANTIN
WKKLFGTSGQKTKIISNRGENSCKATGQVCHALCSPEGCWGPEPRDCVSCRNVSRG
RECVDKCNLLEGEPREFVENSECIQCHPECLPQAMNITCTGRGPDNCIQCAHYIDGP
HCVKTCPAGVMGENNTLVWKYADAGHVCHLCHPNCTYGCTGPGLEGCPTNGPKIP
S: ECD of cyEGFR

Amino acid sequence full length human cMET insert for expression on the cell surface (Identical to GenBank: P08581-2). The sequence differs from the reference sequence at position with an insertion at 755-755: S→STWWKEPLNIVSFLFCFAS

MKAPAVLAPGILVLLFTLVQRSNGECKEALAKSEMNVNMKYQLPNFTAETPIQNVIL
HEHHIFLGATNYIYVLNEEDLQKVAEYKTGPVLEHPDCFPCQDCSSKANLSGGVWK
DNINMALVVDTYYDDQLISCGSVNRGTCQRHVFPHNHTADIQSEVHCIFSPQIEEPSQ
CPDCVVSALGAKVLSSVKDRFINFFVGNTINSSYFPDHPLHSISVRRLKETKDGFMFL
TDQSYIDVLPEFRDSYPIKYVHAFESNNFIYFLTVQRETLDAQTFHTRIIRFCSINSGL
HSYMEMPLECILTEKRKKRSTKKEVFNILQAAYVSKPGAQLARQIGASLNDDILFGVF
AQSKPDSAEPMDRSAMCAFPIKYVNDFFNKIVNKNNVRCLQHFYGPNHEHCFNRTL
LRNSSGCEARRDEYRTEFTTALQRVDLFMGQFSEVLLTSISTFIKGDLTIANLGTSEG
RFMQVVVSRSGPSTPHVNFLLDSHPVSPEVIVEHTLNQNGYTLVITGKKITKIPLNGL
GCRHFQSCSQCLSAPPFVQCGWCHDKCVRSEECLSGTWTQQICLPAIYKVFPNSAPL
EGGTRLTICGWDFGFRRNNKFDLKKTRVLLGNESCTLTLSESTMNTLKCTVGPAMN
KHFNMSIIISNGHGTTQYSTFSYVDPVITSISPKYGPMAGGTLLTLTGNYLNSGNSRHI
SIGGKTCTLKSVSNSILECYTPAQTISTEFAVKLKIDLANRETSIFSYREDPIVYEIHPT
KSFISTWWKEPLNIVSFLFCFASGGSTITGVGKNLNSVSVPRMVINVHEAGRNFTVAC
QHRSNSEIICCTTPSLQQLNLQLPLKTKAFFMLDGILSKYFDLIYVHNPVFKPFEKPV
MISMGNENVLEIKGNDIDPEAVKGEVLKVGNKSCENIHLHSEAVLCTVPNDLLKLNS
ELNIEWKQAISSTVLGKVIVQPDQNFTGLIAGVVSISTALLLLLGFFLWLKKRKQIKDL
GSELVRYDARVHTPHLDRLVSARSVSPTTEMVSNESVDYRATFPEDQFPNSSQNGSC
RQVQYPLTDMSPILTSGDSDISSPLLQNTVHIDLSALNPELVQAVQHVVIGPSSLIVHF
NEVIGRGHFGCVYHGTLLDNDGKKIHCAVKSLNRITDIGEVSQFLTEGIIMKDFSHPN
VLSLLGICLRSEGSPLVVLPYMKHGDLRNFIRNETHNPTVKDLIGFGLQVAKGMKYL
ASKKFVHRDLAARNCMLDEKFTVKVADFGLARDMYDKEYYSVHNKTGAKLPVKW
MALESLQTQKFTTKSDVWSFGVLLWELMTRGAPPYPDVNTFDITVYLLQGRRLLQP
EYCPDPLYEVMLKCWHPKAEMRPSFSELVSRISAIFSTFIGEHYVHVNATYVNVKCV
APYPSLLSSEDNADDEVDTRPASFWETS

Of which:

-MKAPAVLAPGILVLLFTLVQRSNG: signal peptide
-ECKEALAKSEMNVNMKYQLPNFTAETPIQNVILHEHHIFLGATNYIYVLNEE
DLQKVAEYKTGPVLEHPDCFPCQDCSSKANLSGGVWKDNINMALVVDTYYDDQLIS
CGSVNRGTCQRHVFPHNHTADIQSEVHCIFSPQIEEPSQCPDCVVSALGAKVLSSVK
DRFINFFVGNTINSSYFPDHPLHSISVRRLKETKDGFMFLTDQSYIDVLPEFRDSYPIK
YVHAFESNNFIYFLTVQRETLDAQTFHTRIIRFCSINSGLHSYMEMPLECILTEKRKK
RSTKKEVFNILQAAYVSKPGAQLARQIGASLNDDILFGVFAQSKPDSAEPMDRSAMC
AFPIKYVNDFFNKIVNKNNVRCLQHFYGPNHEHCFNRTLLRNSSGCEARRDEYRTE
FTTALQRVDLFMGQFSEVLLTSISTFIKGDLTIANLGTSEGRFMQVVVSRSGPSTPHV
NFLLDSHPVSPEVIVEHTLNQNGYTLVITGKKITKIPLNGLGCRHFQSCSQCLSAPPF
VQCGWCHDKCVRSEECLSGTWTQQICLPAIYKVFPNSAPLEGGTRLTICGWDFGFRR
NNKFDLKKTRVLLGNESCTLTLSESTMNTLKCTVGPAMNKHFNMSIIISNGHGTTQY
STFSYVDPVITSISPKYGPMAGGTLLTLTGNYLNSGNSRHISIGGKTCTLKSVSNSILE
CYTPAQTISTEFAVKLKIDLANRETSIFSYREDPIVYEIHPTKSFISGGSTITGVGKNLN
SVSVPRMVINVHEAGRNFTVACQHRSNSEIICCTTPSLQQLNLQLPLKTKAFFMLDGI
LSKYFDLIYVHNPVFKPFEKPVMISMGNENVLEIKGNDIDPEAVKGEVLKVGNKSCE
NIHLHSEAVLCTVPNDLLKLNSELNIEWKQAISSTVLGKVIVQPDQNFT: ECD of
human cMET
-GLIAGVVSISTALLLLLGFFLWL: transmembrane region
-KKRKQIKDLGSELVRYDARVHTPHLDRLVSARSVSPTTEMVSNESVDYRATF
PEDQFPNSSQNGSCRQVQYPLTDMSPILTSGDSDISSPLLQNTVHIDLSALNPELVQA
VQHVVIGPSSLIVHFNEVIGRGHFGCVYHGTLLDNDGKKIHCAVKSLNRITDIGEVSQ
FLTEGIIMKDFSHPNVLSLLGICLRSEGSPLVVLPYMKHGDLRNFIRNETHNPTVKDL
IGFGLQVAKGMKYASKKFVHRDLAARNCMLDEKFTVKVADFGLARDMYDKEYYSV
HNKTGAKLPVKWMALESLQTQKFTTKSDVWSFGVLLWELMTRGAPPYPDVNTFDIT
VYLLQGRRLLQPEYCPDPLYEVMLKCWHPKAEMRPSFSELVSRISAIFSTFIGEHYVH
VNATYVNVKCVAPYPSLLSSEDNADDEVDTRPASFWETS: intracellular region

### Reference Antibodies

Anti-cMET Antibodies are known in the art (Table 1). Monospecific bivalent cMET antibodies were constructed according to published information and expressed in 293F Freestyle cell. Table 1 shows the related disclosed information. Monospecific bivalent antibodies directed against cMET were constructed according to published information and expressed in 293F Freestyle cells. For HGF ligand blocking assays VH- and VL-encoding gene segments of patent-derived anti-cMET antibodies were re-cloned in a phage display vector for display on filamentous bacteriophage.

Reference antibody cetuximab (Erbitux) was used as reference antibody for the EGFR Fab panel.

2994 Fab protein was generated from purified PG2994 IgG by papain digestion. Therefore PG2994 was incubated with papain coupled on beads (Pierce #44985), and allowed to digest for 5.5 hour at 37° C. under rotation. Fab fragments were purified from the digestion mixture by filtration over MabSelectSure LX. Flow through fractions containing Fab protein, concentrated to 3 ml using vivaspin20 10 kDa and further purified by gel filtration using a superdex75 16/600 column in PBS.

### Example 2

### Generation of Bivalent Monoclonal Antibodies and Antibody Characterization

VH genes of unique antibodies, as judged by VH gene sequence and some sequence variants thereof, were cloned in the backbone IgG1 vector. Suspension adapted 293F Freestyle cells were cultivated in T125 flasks at a shaker plateau until a density of 3.0×10⁶ cells/ml. Cells were seeded at a density of 0.3-0.5×10⁶ viable cells/ml in each well of a 24-deep well plate. The cells were transiently transfected with the individual sterile DNA: PEl mixture and further cultivated. Seven days after transfection, supernatant was harvested and filtrated through 0.22 μM (Sartorius) and purified on protein A beads using batch purification followed by a buffer exchange to PBS.

Cross Block Assay cMET Antibodies

cMET specific phages were tested for competition with cMET reference antibodies in ELISA. Therefore 2.5 μg/ml of cMET-Fc fusion protein was coated overnight to MAXISORP™ ELISA plates at 4° C. Wells of the ELISA plates were blocked with PBS (pH 7.2) containing 2% ELK for 1H at RT while shaking (700 rpm). Next reference or negative control IgG was added at a concentration of 5 μg/ml and allowed to bind for 15 min at RT at 700 rpm. Next, 5 μl of PEG precipitated phage was added and allowed to bind for 1H at RT at 700 rpm. Bound phages were detected with HRP labelled anti-M13 antibody for 1H at RT at 700 rpm. As a control the procedure was performed simultaneously with an antibody specific for the coated antigens and a negative control phage. Bound secondary antibody was visualized by TMB/H₂O₂ staining and staining was quantified by means of OD₄₅₀ₙₘ measurement. Table 2 demonstrates that MF4040 and MF4356 show competition with the 5D5 reference antibody. MF4297 competes with 13.3.2 and C8H241 to a lesser extent. The positive control phages all show complete competition with the corresponding IgG, whereas the no antibody control, does not influence the competition assay.

### Generation of Bispecific Antibodies

Bispecific antibodies were generated by transient co-transfection of two plasmids encoding IgG with different VH domains, using a proprietary CH3 engineering technology to ensure efficient heterodimerisation and formation of bispecific antibodies. The common light chain is also co-transfected in the same cell, either on the same plasmid or on another plasmid. In our co-pending applications (e.g. WO2013/157954 and WO2013/157953; incorporated herein by reference) we have disclosed methods and means for producing bispecific antibodies from a single cell, whereby means are provided that favor the formation of bispecific antibodies over the formation of monospecific antibodies. These methods can also be favorably employed in the present invention. Specifically, preferred mutations to produce essentially only bispecific full length IgG molecules are amino acid substitutions at positions 351 and 366, e.g. L351K and T366K (numbering according to EU numbering) in the first CH3 domain (the ‘KK-variant’ heavy chain) and amino acid substitutions at positions 351 and 368, e.g. L351D and 10 L368E in the second CH3 domain (the ‘DE-variant’ heavy chain), or vice versa. It was previously demonstrated in our co-pending applications that the negatively charged DE-variant heavy chain and positively charged KK-variant heavy chain preferentially pair to form heterodimers (so-called ‘DEKK’ bispecific molecules). Homodimerization of DE-variant heavy chains (DE-DE homodimers) or KK-variant heavy chains (KK-KK homodimers) are disfavored due to strong repulsion between the charged residues in the CH3-CH3 interface between identical heavy chains.

Table 3 shows which cMET and EGFR Fab arms were cloned in the appropriate KK and DE vectors. After production, bispecific IgG were purified by protein-A batch purification and the buffer was exchanged to PBS. Successful productions resulted in an IgG1 full length antibody, with a minimal concentration of 0.1 mg/ml, which were assigned a unique code (PBnnnnn; where nnnnn represents a randomly generated number) to identify the specific combination of 2 different target binding Fab fragments. Successfully produced bispecific IgG were tested for binding to their respective targets in ELISA. Reference is made herein to PCTNL/2018/050537 (published as WO2019/031965) for more details on production of bispecific antibodies.

### Example 3

Screening of c-MET×EGFR Bispecific Antibodies in an EGF/HGF and HGF and EGF Proliferation Assay

The potency of a panel of cMET x EGFR bispecific antibodies was tested in N87 cells using an HGF/EGF, HGF and EGF assays. The N87 cell line, official name NCI-N87, is a gastric carcinoma cell line derived from a metastatic site and has high EGFR expression levels and intermediate cMET expression levels (Zhang et al, 2010). Antibodies were tested in an 8 steps semi-log titration ranging from 10 μg/ml to 3.16 ng/ml. Each antibody was tested in duplicate. The anti-RSV-G antibody PG2708 was used as negative control. The reference antibody 2994 Fab was used as positive control for the HGF assay and the reference antibody cetuximab was used as positive control for the EGF assay.

An equimolar 1:1 cetuximab/5D5 Fab was used as positive control for the EGF, HGF and EGF/HGF assays.

Wells with either one, or a combination of ligand, as well as medium control were included to determine the assay window. Antibodies were diluted in chemically defined starvation medium (CDS: RPMI1640 medium, containing 80U penicillin and 80 μg of streptomycin per ml, 0.05% (w/v) BSA and 10 μg/ml holo-transferrin) and 50 μl of diluted antibody was added to the wells of a 96 wells black well clear bottom plate (Costar). Ligand was added (50 μl per well of a stock solution containing 400 ng/ml HGF and 4 ng/ml of EGF, and a EGF/HGF concentration of 4 ng/ml EGF/400 ng/ml HGF diluted in CDS: R&D systems, cat. nr. 396-HB and 236-EG). N87 cells were trypsinised, harvested and counted and 8000 cells in 100 μl of CDS were added to each well of the plate. To avoid edge effects, plates were left for an hour at RT before being put in a container inside a 37° C. cell culture incubator for three days. On the fourth day, Alamar blue (Invitrogen, #DAL1100) was added (20 μl per well) and the fluorescence was measured after 6 hours of incubation (at 37° C.) with Alamar blue using 560 nm excitation and 590 nm readout on a Biotek Synergy 2 Multi-mode microplate reader. Fluorescence values were normalised to uninhibited growth (no antibody, but both ligands added).

Table 4 lists the results of the various experiments. In the N87 HGF/EGF assay, fourteen different cMET×EGFR bispecifics with potency comparable to the reference monospecific antibodies (equimolar mix of cetuximab and 5D5 Fab) were identified: PB7679, PB7686, PB8218, PB8244, PB8292, PB8316, PB8340, PB8364, PB8388, PB8511, PB8535, PB8583, PB8607 and PB8640.

In the N87 EGF assay, eleven different cMET×EGFR bispecifics with potency comparable to monospecific cetuximab were identified: PB7679, PB8244, PB8292, PB8340, PB8364, PB8388, PB8511, PB8535, PB8583, PB8607 and PB8640. They all contain the EGFR Fab arm MF3755. In the HGF N87 assay nine bispecifics were identified that showed a higher potency compared to the monospecific 5D5 Fab reference antibody: PB8218, PB8388, PB8511, PB8532, PB8535, PB8545, PB8583, PB8639 and PB8640. They contain six different cMET Fab arms MF4040, MF4297, MF4301, MF4356, MF4491 and MF4506.

### ADCC Activity

The ADCC activity of the 24 cMet×EGFR bispecifics was tested to the tumor cell lines N87 (EGFR-high, cMET-low) and MKN-45 (EGFR-low, cMET-amplified). The ADCC assay was performed using the Promega ADCC Bioassay kit in 384-well plate format. Antibodies were tested in duplicate at 9 different concentrations in semi-log serial dilutions ranging from 10 μg/ml to 1 ng/ml.

The reference cetuximab antibody was included as a positive control for the assay and PG2708 was used as negative control antibody. Antibodies or assay medium control (no IgG) were incubated for 6 hours of induction at 37° C. with ADCC effector cells, and target cells (N87 or MKN-45). Luciferase activity was quantified using Bio-Glo luciferase reagent.

None of the cMET×EGFR bispecifics showed a significant ADCC activity in both cell lines. The positive control reference cetuximab antibody showed a dose-dependent ADCC activity to both cell lines.

Five bispecifics composed of EGFR and cMet arms which did show high efficacy in the N87 HGF/EGF assay and showed high sequence diversity (Table 5) were selected for further analysis. Two from the five bispecifics contain MF4356, which competes with 5D5 for binding to cMET (Table 2). Table 5 summarizes the characteristics of the selected candidates.

### Example 4

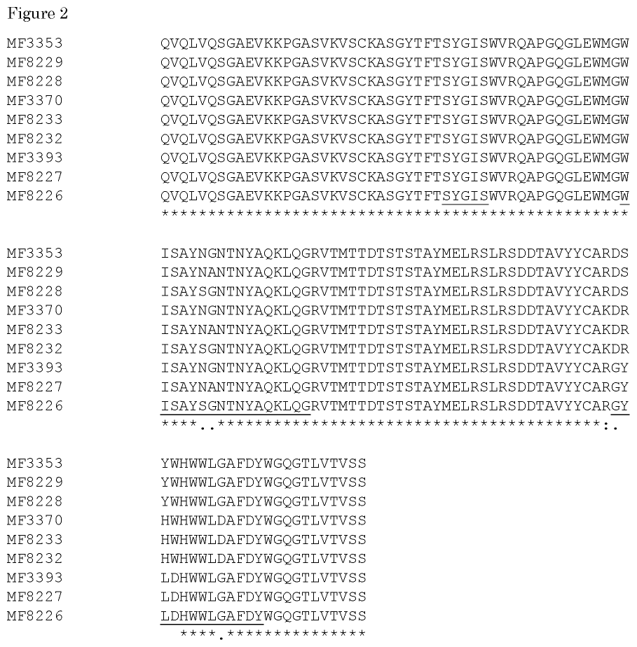

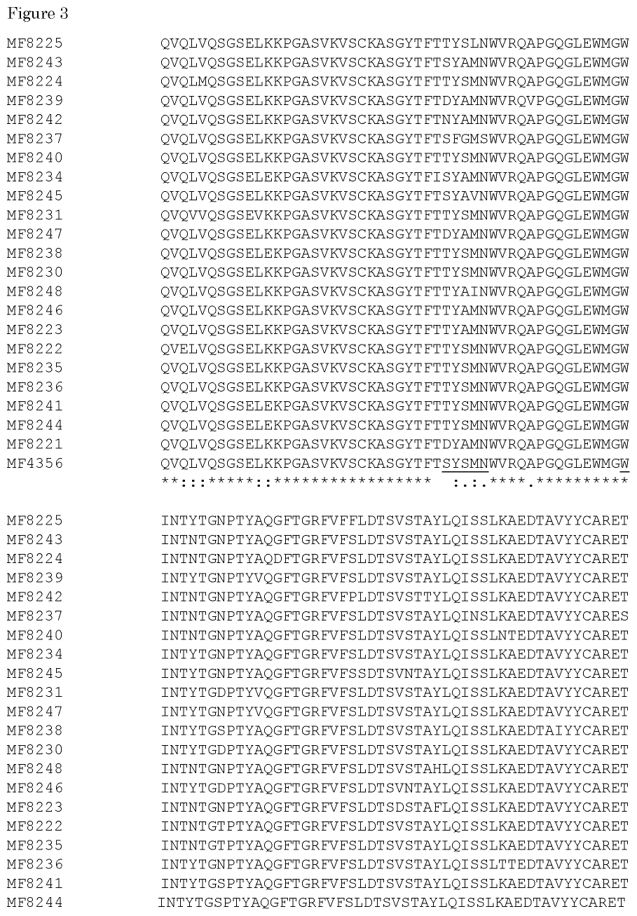

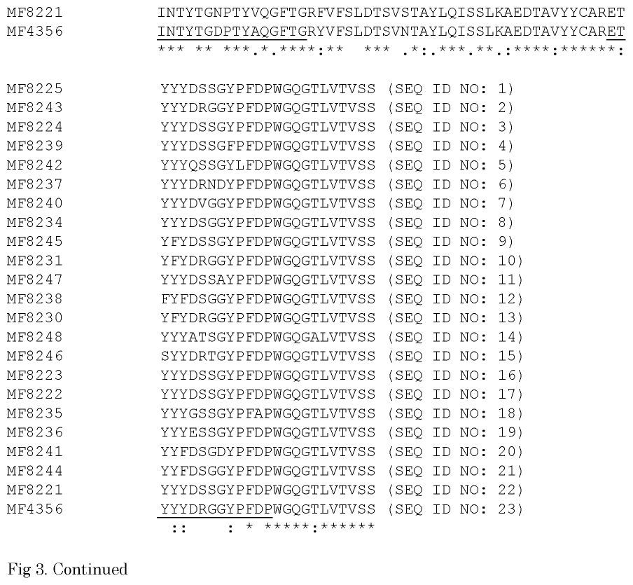

FIG. 2 depicts various sequences for alternative variable regions of the heavy chain of an EGFR binding variable domain as disclosed herein. FIG. 3 depicts various sequences for alternative variable regions of the heavy chain of a cMET binding variable domain as disclosed herein. The heavy chain variable regions were used to create a number of different cMET x EGFR bispecific antibodies. The light chain in these antibodies has the sequence as depicted in FIG. 4B. Bispecific antibodies were produced as described in example 1. The antibodies were also produced as an ADCC enhanced version. ADCC enhanced versions were produced by including in the co-transfection of the antibody constructs, a DNA encoding a reductase enzyme that removes a fucose residue from the Fc region of IgG1. See table 6 for a list of bispecific antibodies used and their PB coding.

### Example 5

The heavy chain variable region (VH) of the cMET variable domain of PB8532 comprises the amino acid of MF4356 as depicted for instance in FIG. 3. The VH of the cMET variable domain of PB19748 comprises the amino acid sequence of MF8230 (see FIG. 3). The VH of the EGFR variable domain of PB8532 comprises the amino acid of MF3370 as depicted for instance in FIG. 2. The VH of the EGFR variable domain of PB19748 comprises the amino acid sequence of MF8233 of FIG. 2. The light chain in PB8532 and PB19748 is the same and is depicted in FIG. 4B. The cMET antibody LY2875358 antibody is among other described in Kim and Kim 2017.

### Example 6

Efficiency of bispecific antibody PB19748 in EGFR exon 20 insertion models

The aim of this study was to evaluate the anti-tumor efficacy of PB19748 in patient-derived tumor xenograft (PDX) non-small cells lung cancer (NSCLC) model with EGFR exon 20 insertions. The EGFR exon 20 insertions (“EGFRex20ins”) represent a class that encode mutants with amino acid insertions clustered between positions 762 and 774 that result in constitutive activation of EGFR. EGFR exon 20 insertions confer resistance to approved EGFR TKIs in human subjects with cancer haboring such mutations and are associated with poor prognosis.

### Material

PB19748 was generated by Merus and control material was the PB19748 vehicle and consisted of 12% of the PB19748 formulation buffer without antibody, although other negative controls such as saline or PBS may also be used.

### PDX Model Characteristics

The model LXFE2478 was generated at Charles River in nude mice which have functional Fc-effector cells.

This model carries mutation EGFRex20ins (M766_A767insASV). It also carries a point mutation (E168D) in the SEMA domain of c-MET, which is located at the ligand binding site of c-MET.

Insertion of 9 nucleotides in exon 20 in this model affects the EGFR tyrosine kinase domain (M766X) and confers resistance to small molecule EGFR inhibitors. This model carries no mutations in B-RAF, H-/N- and KRAS and PTEN genes (whole exome sequencing was done for both patient tumor and tumor xenograft with matching results).

Expression of EGFR, c-MET and HGF in LXFE2478 PDX Model

The model LXFE2478 was analysed for expression of EGFR and c-MET receptors and ligand huHGF, in order to investigate its suitability for in vivo studies.

Two tumor pieces were evaluated for expression of the EGFR and c-MET receptors, and that of the ligand huHGF, via western blot. huHGF was included in the analysis. Tumor samples obtained from the PDX model were assessed by Simple Western Size technology (SWS) to confirm expression of EGFR, HGF, and c-MET.

Western blotting revealed that LXFE2478 model expressed EGFR, HGF and MET on protein level (FIG. 6 and Table 7).

### Statistical Methods:

Antitumor efficacy of all groups was assessed using the control vehicle/placebo buffer group as a reference. Tumor growth inhibition was determined by the comparison of RTVs of the test groups with the control group and is expressed as minimum T/C value in percent. For the evaluation of the statistical significance of antitumor efficacy, the non-parametric Kruskal-Wallis test followed by Dunn's method for multiple comparisons was performed. Individual RTVs of test and control groups were compared on days on which the minimum T/C values were achieved in the test groups. Statistical analysis was only carried out if at least 50% of the initially randomized animals still remained in the relevant group. Comparisons between test groups were carried out for the same days. All p-values <0.05 were considered statistically significant. Statistical calculations were performed using GraphPad Prism bioanalytic software (version 9.10 for Microsoft Windows, GraphPad Software, San Diego, California, USA.

### Treatment Schedule and Methods:

FIG. 7 shows a schematic overview of the treatment schedule

Tumors were harvested from the donor PDX mice, they were cut into fragments (LXFE2478: 3-4 mm edge length) and inoculated into recipient nude mice subcutaneously (SC) in the flank. When the tumor implants reached approximately 80-200 mm3 in a sufficient number of animals, mice were assigned to 5 groups with 5 mice per group. Randomization was performed based on “stratified distribution” method. Treatment started on the same day as randomization (day 0).

Antibody was administered once a week intra peritoneally (IP) at 0.5 mg/kg, 2.5 mg/kg, 8 mg/kg, and 25 mg/kg dose over a period of 5 weeks. Mice in group 1 were treated with vehicle 1 once a week for 5 weeks every day for 30 days. The treatment plan is shown in Table 8.

After randomization, animals were routinely monitored for morbidity and mortality, they were weighted twice a week, and tumor volumes (TV) were determined twice a week with a caliper. Relative body weight (RBW) was calculated by dividing the absolute weight or volume on a certain day by the absolute weight at 0 multiplied by 100. Relative tumor volumes (RTV) were calculated by dividing the absolute individual tumor volume on a certain day by the absolute tumor volume at day 0 multiplied by 100.

For mice that lost >10% body weight, weight was measured daily, and animals had facilitated access to feed and water, and access to DietGel. For mice that lost >15% body weight, therapy was suspended until they regained a RBW of ≥90%.

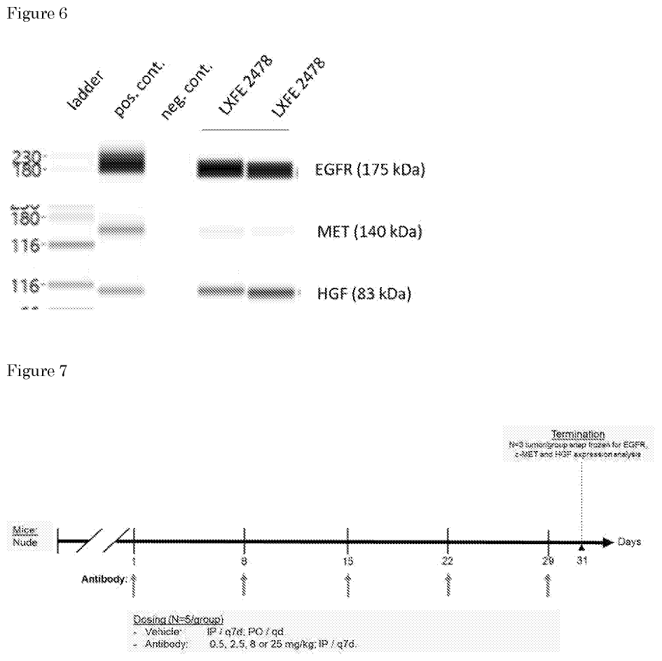

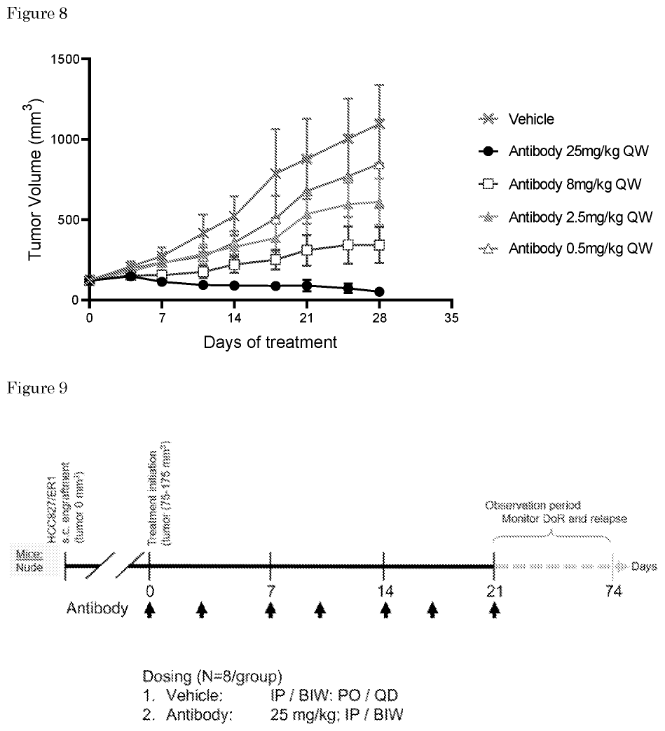

Monotherapy with bispecific antibody PB19478 showed a dose dependent anti-tumor efficacy (FIG. 8). Monotherapy at 25 mg/kg dose of antibody led to tumor remission (min. T/C value 2.7%) and was significantly more efficacious than the treatment with vehicle and the treatment with 0.5 mg/kg or 2.5 mg/kg dose. Treatment at 0.5 mg/kg and 2.5 mg/kg dose showed tumor partial regression.

Monotherapy correlates with low toxicity in mice, demonstrated by negligible body weight loss and 100% adjusted survival rate in all groups.

EGFR exon20ins-mutated cancer is generally refractory to EGFR tyrosine kinase inhibitors and is associated with poor prognosis.

### Example 7

Efficiency of bispecific antibody PB19478 in EGFR exon 19 deletion NSCLC model. The aim of this study was to evaluate the anti-tumor efficacy of antibody PB19478 in a cell line-derived tumor xenograft (CDX) non-small cell lung cancer (NSCLC) model with an EGFR exon 19 deletion. This adenocarcinoma cell line, HCC827-ER1, harbors an activating EGFR mutation in exon 19 (deletion E746-A750), and is resistant to approved EGFR TKIs such as erlotinib.

Material: PB19748 was generated by Merus as mentioned in Example 5.

### CDX Model Characteristics:

The model HCC827-ER1 was generated in BALB/c nude mice. It carries an EGFR mutation in exon 19 (deletion E746-A750), and has amplified c-MET copy number and Axl expression compared to the wild type HCC827 cell line. The HCC827-ER1 cell line is resistant to EGFR TKI erlotinib and was generated via repeated in vitro exposure of the wild type cell line to escalating concentrations of erlotinib.

### Experimental Procedures

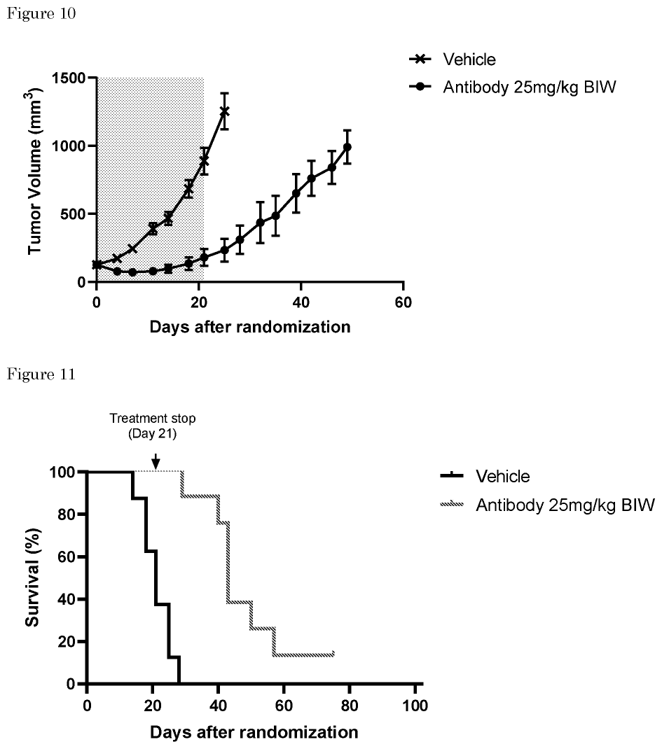

Each mouse was inoculated in the right front flank region with HCC827-ER1 tumor cells mixed with Matrigel for tumor development. The randomization started when the mean tumor size reached approximately 125 (75-175) mm3. Totally 16 tumor-bearing mice were enrolled in the tumor efficacy study and randomly allocated to 2 different groups, as shown in table 9, with 8 mice per group. The date of randomization was denoted as day 0 and dosing was initiated from day 0. Mice were treated with antibody for 3 weeks, followed by a dose-free observation period of up to 74 days. The dose-free observation period was included to compare duration of response and relapse (tumor re-growth) after treatment end. Mice with tumor volume exceeding 1500 mm3, or with body weight loss over 20% relative to the weight at the first day of treatment were terminated during the treatment period. Antibody was administered twice a week intra peritoneally (i.p.) at 25 mg/kg dose over a period of 21 days, with a total of 7 doses. Eight mice in group 1 were treated with vehicle control (placebo buffer). A schematic overview of the treatment schedules is shown in FIG. 9. Tumor volume (mm3) was measured twice per week in 2 dimensions using a caliper. The animals were checked for any effects of tumor growth and treatments on behavior such as mobility, food and water consumption, body weight gain/loss, eye/hair matting and any other abnormalities. Body weights were measured twice per week after randomization. None of the mice lost more than 15% body weight, obviating any need to suspend administration of a therapeutic agent in any of the treatment Groups. FIG. 10 shows the effects of bispecific antibody PB19478 on tumor volume in NSCLC CDX model during the complete observation period with treatment stop indicated on day 21. Antibody treatment showed tumor regression compared to the vehicle control group. All treatments were well tolerated. No mice in the study showed body weight loss >10% from starting body weight. In fact, no statistically significant differences in absolute body weight were observed between Groups 1 and 2 on day 21, which was the last treatment day. Absolute body weight of mice in a further treatment Group (i.e. 25 mg/kg osimertinib, once a day administration per oral gauge) was lower compared to Groups 1 and 2 (P=0.0167 vs Group 2, data not shown). Apart from tumor scabbing or tumor ulceration in some mice in Groups 1 and 2, no adverse events were observed. Animal survival curves are shown in FIG. 11. Log-rank (Mantel-Cox) test of combination vs. each corresponding monotherapy group is significant as follows: antibody 25 mg/kg vs. vehicle P=0.0001.

In summary, these results show preclinical antitumor activity of the antibody under investigation against an NSCLC CDX model harboring a mutation in exon 19 (deletion E746-A750).

### Example 8

In the dose escalation phase, bispecific antibody PB19478 will be administered with increasing doses to patients with NSCLC harboring an activating EGFR mutation (TKI sensitizing mutations and/or approved TKI-resistance mutations) or an activating c-MET mutation (exon 14 skipping)/amplification (MET/CEP7>5 or cfDNA≥2 copies), patients with GC/GEJ adenocarcinoma with an activating EGFR amplification (EGFR/CEP7≥2 or cfDNA≥8 copies) or c-MET amplification (MET/CEP7>5 or cfDNA≥2 copies), or patients with HNSCC or ESCC who in all cases, have progressed after receiving prior therapy for advanced/metastatic disease.

Allometric scaling of a preclinical PK model was used to predict antibody exposure in humans. The antibody starting dose is 100 mg (flat dose, intravenously) once every 2 weeks (q2w), with 4-week cycles (28 days). Five dose levels between: 100-3000 mg are planned to be investigated.

Cohorts of patients will be treated with antibody until the MTD is reached or a lower recommended dose(s) is established.

The RP2D (recommended phase 2 dosing) is defined as the dose at or below the MTD, taking into account available data for PK, pharmacodynamic activity, and preliminary antitumor activity.

### Dose Expansion

Using an RP2D of 1500 mg, the planned expansion cohorts in the phase 2 part can be initiated. The safety of the RP2D will be confirmed during dose expansion in the first 12 patients treated with antibody alone, for at least 2 cycles (recruitment will continue in the meanwhile). Antitumor activity of antibody (alone or in combination with osimertinib) will be evaluated in terms of ORR, and an evaluation of other efficacy parameters, safety, tolerability, PK, immunogenicity, and biomarkers will be performed. The following cohorts of locally advanced unresectable/metastatic solid tumors may be opened:
Cohort A: PB19478 monotherapy: NSCLC patients may harbor EGFR exon 20 insertion mutations (first line [1L] and >second line [2L] progressed after first line chemotherapy). Results from patients with NSCLC are described in Example 9.
Cohort B: PB19478 monotherapy: NSCLC patients may harbor cMet exon 14 mutations in a ≥2 L, capmatinib and tepotinib resistant population, progressed after an approved c-Met TKI inhibitor.
Cohort C: PB19478 monotherapy: Patients with GC/GEJ and HNSCC that may harbor an EGFR or a cMet driving mutation. Results from a patient with HNSCC is described in Example 10.

### Study Population

### Inclusion Criteria

Patients must fulfill all of the following requirements to enter the study:
1. Signed informed consent before initiation of any study procedures.
2. Age ≥18 years at signature of informed consent.
3. Histologically or cytologically confirmed solid tumors with evidence of metastatic or locally advanced unresected disease that is incurable.

1. Dose Escalation Part-Patients who have failed prior standard first-line treatment. Patients must have progressed on or be intolerant to therapies that are known to provide clinical benefit. There is no limit to the number of prior treatment regimens. Patients must have:
Non-small cell lung cancer (NSCLC) harboring activating EGFR mutations including tyrosine kinase inhibitor (TKI) sensitizing mutations (e.g., 19del and L858R), and/or approved TKI-resistance mutations (e.g., acquired TKI-resistance mutations, i.e., T790M, C797S, L792, L798I, exon 20 insertion), or any activating c-MET mutation/amplification (e.g., high-level c-MET amplification [MET/CEP7>5 or cfDNA≥2 copies], or c-MET exon 14 skipping mutation).
   *NOTE: Patient identification will be based on previous treatment history with EGFR tyrosine kinase inhibitors and on local tests performed in CLIA-certified laboratories.

2. Cohort Expansion Part—For ≥2 L patients must have progressed on or be intolerant to therapies that are known to provide clinical benefit. There is no limit to the number of prior treatment regimens.

Patients Must have:
Cohort A: NSCLC harboring an EGFR exon 20 insertion, that has progressed after platinum doublets (a limited number of first line patients can be included).
Cohort B: NSCLC harboring cMet exon 14 skipping mutation ≥2 L, capmatinib and tepotinib resistant population or treated with a cMET TKI or cMET TKI naive.
Cohort C: Selected solid tumors (including GC/GEJ, HNSCC, e.g. HNSCC refractory to approved treatment (regardless of driving mutations), or ESCC) harboring an EGFR or cMet driving mutation, or cMET alteration, NSCLC patients with rare EGFR mutations (G719X, L861Q, and S768I) not otherwise included in the other cohorts. Patients exhausted or be intolerant to all approved therapies with proven clinical benefit or have no available, approved, therapeutic options.
Cohort F: NSCLC harboring a c-MET exon 14 skipping mutation, treatment naïve for advanced disease.

4. Having availability of FFPE embedded after progression at the latest therapy, archival or a fresh tumor tissue sample.

5. Measurable disease as defined by RECIST version 1.1 by radiologic methods (patients with non-measurable but evaluable disease can be included in the dose escalation part).

6. Eastern Cooperative Oncology Group (ECOG) performance status of 0 or 1.

7. Life expectancy ≥12 weeks, as per Investigator judgment 8. Adequate organ function, as per Investigator judgment.
Absolute neutrophil count (ANC) ≥1.5×10⁹/L.
Hemoglobin ≥9 g/dL
Platelets ≥100×10⁹/L
Corrected total serum calcium within normal ranges.
Serum magnesium within normal ranges (or corrected with supplements).
Serum potassium within normal ranges.
Alanine aminotransferase (ALT), aspartate aminotransferase (AST)≤3×upper limit of normal (ULN) and total bilirubin≤1.5×ULN (patients with Gilbert's syndrome are eligible if conjugated bilirubin value is within normal limits); in cases of liver involvement, ALT/AST≤5×ULN and total bilirubin≤2×ULN will be allowed
Serum creatinine≤1.5×ULN or creatinine clearance ≥50 mL/min calculated according to the Cockroft and Gault formula or MDRD formula for patients aged >65 years.
Serum albumin >3.3 g/dL

### Exclusion Criteria

The presence of any of the following criteria excludes a patient from participating in the study:

1. Central nervous system metastases that (not exclusionary in escalation, mandatory in expansion):
Are untreated or symptomatic (patients with untreated, asymptomatic lesions can be included if in the investigator judgment considered stable)
require radiation or surgery
require continued steroid therapy (>10 mg prednisone or equivalent) to control symptoms within 14 days of study entry.

2. Known leptomeningeal involvement.

3. Participation in another clinical study or treatment with any investigational drug within 4 weeks prior to study entry.

4. Systemic anticancer therapy or immunotherapy within 4 weeks or 5 half-lives, whichever is shorter, of the first dose of study drug. For cytotoxic agents that have major delayed toxicity (e.g., mitomycin C, nitrosoureas), a washout period of 6 weeks is required. Note: For agents with long half-lives, enrollment before the fifth half-life requires Sponsor approval.

5. Major surgery or radiotherapy within 3 weeks of the first dose of study drug. Patients who received prior radiotherapy to >25% of bone marrow at any time are not eligible.

6. Persistent grade >1 clinically significant toxicities, in the Investigator judgment, related to prior antineoplastic therapies (except for alopecia); stable sensory neuropathy ≤grade 2 NCI-CTCAE v5.0 and hypothyroidism ≤grade 2 which is stable on hormone replacement are allowed.

7. History of hypersensitivity reaction or any toxicity attributed to human proteins or any of the excipients that warranted permanent cessation of these agents.

8. History of clinically significant cardiovascular disease including, but not limited to:
Prolonged QT interval >480 msec, obtained from 3 electrocardiograms (ECGs), or clinically significant cardiac arrythmia or electrophysiologic disease (i.e., placement of implantable cardioverter defibrillator or atrial fibrillation with uncontrolled rate), or any factors that increase the risk of QTc prolongation or risk of arrhythmic events such as electrolyte abnormalities. Patients with cardiac pacemakers who are clinically stable are eligible.
Heart failure, congenital long QT syndrome, family history of long QT syndrome, or unexplained sudden death under 40 years of age in first-degree relatives or any concomitant medication known to prolong the QT interval and cause Torsades de Pointes.
Uncontrolled (persistent) arterial hypertension: systolic blood pressure >180 mm Hg and/or diastolic blood pressure >100 mm Hg.
Congestive heart failure (CHF) defined as New York Heart Association (NYHA) class III-IV or hospitalization for CHF within 6 months of the first dose of study drug.

9. History of interstitial lung disease including drug-induced interstitial lung disease, radiation pneumonitis that requires treatment with prolonged steroids or other immune suppressive agents within 1 year.

10. Previous or concurrent malignancy, excluding non-basal cell carcinomas of skin or carcinoma in situ of the uterine cervix, unless the tumor was treated with curative or palliative intent and in the opinion of the Investigator, with Sponsor agreement, the previous or concurrent malignancy condition does not affect the assessment of safety and efficacy of the study drug.

11. Current serious illness or medical conditions including, but not limited to uncontrolled active infection, clinically significant pulmonary, metabolic or psychiatric disorders.

12. Active Hepatitis B infection (HBsAg positive) without receiving antiviral treatment. Note: Patients with active hepatitis B (HbsAg positive) must receive antiviral treatment with lamivudine, tenofovir, entecavir, or other antiviral agents, starting at least >7 days before the initiation of the study treatment. Patients with antecedents of Hepatitis B (anti-HBc positive, HbsAg and HBV-DNA negative) are eligible.

13. Positive test for Hepatitis C ribonucleic acid (HCV RNA); Note: Patients in whom HCV infection resolved spontaneously (positive HCV antibodies without detectable HCV-RNA) or those who achieved a sustained virological response after antiviral treatment and show absence of detectable HCV RNA >6 months (with the use of IFN-free regimens) or ≥12 months (with the use of IFN-based regimens) after cessation of antiviral treatment are eligible.

14. Known history of HIV (HIV 1/2 antibodies). Patients with HIV with undetectable viral load are allowed. HIV testing is not required unless mandated by local health authority or regulations.

15. Sexually active male and female patients of childbearing potential must agree to use one of the following highly effective methods of birth control during the entire duration of the study and for 6 months after final administration of PB19478:
combined (estrogen and progestogen containing) hormonal contraception associated with inhibition of ovulation (oral, intravaginal, transdermal)
progestogen-only hormonal contraception associated with inhibition of ovulation (oral, injectable, implantable)
intrauterine device (IUD)
intrauterine hormone-releasing system (IUS)
bilateral tubal occlusion
vasectomized partner1.
sexual abstinence2

16. Pregnant or breast-feeding women are excluded from this study.

ECOG Performance Status Scoring Grade Definition is as followed in the art, meaning: 0 Fully active, able to carry on all pre-disease performance without restriction. 1 Restricted in physically strenuous activity but ambulatory and able to carry out work of a light or sedentary nature, eg, light housework, office work. 2 Ambulatory and capable of all self-care but unable to carry out any work activities. Up and about more than 50% of waking hours. 3 Capable of only limited self-care, confined to bed or chair more than 50% of waking hours. 4 Completely disabled. Cannot carry on any self-care. Totally confined to bed or chair. 5 Dead.

### Investigational Therapy and Regimen

Antibody will be administered as an IV infusion at a dose level of 1500 mg (flat dose) as the RP2D level, once every 2 weeks (q2w) with 4-week cycles. Dose levels between 100-3000 mg (flat dose) may be explored. Dose escalation will be halted once the RP2D has been reached. The administered dose, dose increments, and frequency of dosing for each patient (including the expansion cohorts) is subject to change based on patient safety, PK and pharmacodynamic data, and upon recommendation of the Sponsor. The Sponsor may recommend the use of an alternate weekly dosing schedule for Cycle 1.

### Treatment Duration

Study treatment will be administered until confirmed progressive disease (as per RECIST v1.1), unacceptable toxicity, withdrawal of consent, patient non-compliance, Investigator decision (e.g., clinical deterioration), or Antibody interruption >6 consecutive weeks.

Patients will be followed up for safety for at least 30 days following the last Antibody infusion and until recovery or stabilization of all related toxicities, and for disease progression and survival status every 3 months for up to 1 year.

### Example 9

In the clinical study of Example 8, clinical efficacy was observed in patients with non-small cell lung cancer with EGFR mutations, including an EGFR exon 20 mutation/insertion, an EGFR exon 21 mutation, such as L858R, and an EGFR exon 19 deletion mutation; and c-MET mutations, including a c-MET exon14 skipping mutation. In more detail, target lesion reductions were observed in several patients, including a confirmed partial response, two cases of stable disease and reduction of lesions (including a patient suffering from a c-MET exon14 mutation), a patient suffering from an exon 19 mutation and a patient suffering from an EGFR amplification mutation. Patients had been prior treated with various different treatments, including platinum-based chemotherapy, osimertinib, pembrolizumab, nivolumab, cetuximab, crizotinib, capmatinib and tepotinib.

### Example 10

In the clinical study of Example 8, clinical efficacy was observed in a 68 year old female patient with head and neck squamous cell carcinoma (HNSCC) who received prior cisplatin and cemiplimab treatment. In more detail, a reduction in target lesions of 20% was observed in said patient who experienced adverse events of only Grade 1/2.

### Example 11

In the clinical study of Example 8, clinically relevant effects were observed in a multitude of patients with non-small cell lung cancer that carried cMET exon 14 skipping mutations after being dosed the bispecific antibody PB19478 at 1500 mg Q2W. Clinically relevant effects included a confirmed partial response as per investigator review under RECIST criteria 1.1 and stable disease. Several patients had received prior anticancer treatment while several other patients were treatment naïve, meaning they received the bispecific antibody of the clinical trial protocol of Example 8 as the first anticancer treatment for a cMET exon 14 skipping NSCLC.

### Example 12

In the clinical study of Example 8, clinically relevant effects were observed in a multitude of patients with non-small cell lung cancer that carried EGFR exon 20 insertion mutations after being dosed the bispecific antibody PB19478 at 1500 mg Q2W. Clinical relevant effects included confirmed partial response as per investigator review under RECIST criteria 1.1 and stable disease. Several patients had received prior anticancer treatment, such as platinum-based chemotherapy.

### CITED ART

Ahmed, M et al. Lack of in Vivo Antibody Dependent Cellular Cytotoxicity with Antibody containing gold particles/Bioconjugate chemistry (2015): 26 812-816. DOI: 10.1021/acs.bioconjchem.5b00139.
Castoldi, R., et al. “A novel bispecific EGFR/Met antibody blocks tumor-promoting phenotypic effects induced by resistance to EGFR inhibition and has potent antitumor activity.” Oncogene 32.50 (2013): 5593-5601.
Eisenhauer et al., New response evaluation criteria in solid tumours: Revised RECIST guideline (version 1.1). European Journal of Cancer 45 (2009) 228-247)
Ferguson K M. Structure-based view of epidermal growth factor receptor regulation. Annu Rev Biophys 2008; 37:353-73.
Kim, George P., and Axel Grothey. “Targeting colorectal cancer with human anti-EGFR monoclonal antibodies: focus on panitumumab.” Biologics 2.2 (2008): 223-228.
Kim, Ki-Hyun and Kim, Hyori. Progress of antibody-based inhibitors of the HGF-cMET axis in cancer therapy. Experimental & Molecular medicine (2017), e307; doi: 10.1038/emm.2017.17)
Moores, Sheri L., et al. “A novel bispecific antibody targeting EGFR and cMet is effective against EGFR inhibitor-resistant lung tumors.” Cancer research 76.13 (2016): 3942-3953.
Oken M M, Creech R H, Tormey D C, Horton J, Davis T E, McFadden E T, Carbone P P. Toxicity and response criteria of the Eastern Cooperative Oncology Group. Am J Clin Oncol. 1982 December; 5(6):649-655. PMID: 7165009.
Robertson S C, Tynan J, Donoghue D J. RTK mutations and human syndromes: when good receptors turn bad. Trends Genet 2000; 16:368.
Yarden Y. The EGFR family and its ligands in human cancer. Signalling mechanisms and therapeutic opportunities. Eur J Cancer 2001; 37 (Suppl 4): S3-S8.

TABLE 1
Reference antibodies with reported specificities
against cMET extracellular domains.

| Name | INN name | Epitope | MOA |
|---|---|---|---|
| 5D5 | MetMAb | Sema Domain | HGF block |
| 13.3.2 | | | |
| 224G11 | | | |
| C8-H241 | LY-2875358 | | HGF block, |
| | | | internalization |
| R13 | 13-MET | | |

TABLE 2
Competition of cMet reference antibodies with cMET
cLC antibodies. Shown are OD450 values. OD450 values
indicate the existence or lack of competition with
the stated antibody. MF4506 was not tested.

| | Competition of phages with reference antibodies | | | | | | |
|---|---|---|---|---|---|---|---|
| MF tested | no IgG | 13.3.2 | 5D5 | R13 | 224G11 | C8H241 | R28 |
| 4040 | 1.915 | 1.818 | 0.066 | 1.608 | 1.907 | 1.979 | 1.787 |
| 4297 | 1.769 | 0.072 | 1.499 | 1.332 | 1.955 | 1.031 | 1.885 |
| 4356 | 2.380 | 2.541 | 0.088 | 2.231 | 2.170 | 1.806 | 1.825 |
| 13.3.2 | 2.172 | 0.311 | 1.934 | 1.988 | 2.221 | 1.893 | 2.129 |
| 5D5 | 1.868 | 1.773 | 0.164 | 1.660 | 2.025 | 2.054 | 2.035 |
| R13 | 1.693 | 1.590 | 1.549 | 0.090 | 1.878 | 0.078 | 1.794 |

TABLE 3
List of the 24 cMETxEGFR bispecifics antibodies selected after dose
dependent titration experiments in a N87 HGF/EGF proliferation assay.
The MF number of the EGFR and cMET arms in each individual PB as well
as their HCDR3 sequence are indicated.

| | PBs MF's | | | |
|---|---|---|---|---|
| PBs | EGFR cMET | | | |
| PB7678 | MF4280 | EGYYETTTYYYNLFDS | MF4298 | KLEPTGYYYYYMDV |
| PB7679 | MF3755 | ERFLEWLHFDY | MF4487 | KTSRYSGYHYYMDV |
| PB7686 | MF3755 | ERFLEWLHFDY | MF4507 | AHYDILTG |
| PB8021 | MF3755 | ERFLEWLHFDY | MF3462 | GKSHYSWDAFDY |
| PB8218 | MF3752 | DRNWGWDFDY | MF4040 | GTYYYGSGSFSTRVFDAFDV |
| PB8244 | MF3755 | ERFLEWLHFDY | MF4044 | QSRRYSGYASYFDY |
| PB8292 | MF3755 | ERFLEWLHFDY | MF4130 | QRRAYSGYNWYFDL |
| PB8301 | MF4280 | EGYYETTTYYYNLFDS | MF4130 | QRRAYSGYNWYFDL |
| PB8316 | MF3755 | ERFLEWLHFDY | MF4293 | RNDFWSGYLFDY |
| PB8339 | MF3752 | DRNWGWDFDY | MF4294 | KTTVGYYYYYMDV |
| PB8340 | MF3755 | ERFLEWLHFDY | MF4294 | KTTVGYYYYYMDV |
| PB8364 | MF3755 | ERFLEWLHFDY | MF4296 | GPELGYYYYYMDI |
| PB8388 | MF3755 | ERFLEWLHFDY | MF4297 | ASSMITFGGVIVSWFDP |
| PB8511 | MF3755 | ERFLEWLHFDY | MF4301 | RVNRYSGYATYFDL |
| PB8532 | MF3370 | DRHWHWWLDAFDY | MF4356 | ETYYYDRGGYPFDP |
| PB8535 | MF3755 | ERFLEWLHFDY | MF4356 | ETYYYDRGGYPFDP |
| PB8545 | MF4281 | GDLFITGTLDY | MF4356 | ETYYYDRGGYPFDP |
| PB8582 | MF3752 | DRNWGWDFDY | MF4491 | RTSRYSGYHYYLDV |
| PB8583 | MF3755 | ERFLEWLHFDY | MF4491 | RTSRYSGYHYYLDV |
| PB8607 | MF3755 | ERFLEWLHFDY | MF4505 | LLYDLFDL |
| PB8639 | MF3752 | DRNWGWDFDY | MF4506 | SIDMATITDAFDI |
| PB8640 | MF3755 | ERFLEWLHFDY | MF4506 | SIDMATITDAFDI |
| PB8687 | MF3752 | DRNWGWDFDY | MF4508 | GTTGNPYYFYYYMDV |
| PB8688 | MF3755 | ERFLEWLHFDY | MF4508 | GTTGNPYYFYYYMDV |

TABLE 4
Summary of the antibody titration experiments done
using N87 HGF/EGF, HGF and EGF proliferation assays
with the 24 cMETxEGFR bispecific antibodies.

| | | | N87 proliferation assays | | |
|---|---|---|---|---|---|
| | PBs MF′s | | HGF/ | | |
| PBs | EGFR | cMET | EGF | HGF | EGF |
| PB7678 | MF4280 | MF4298 | + | + | + |
| PB7679 | MF3755 | MF4487 | ++ | − | ++ |
| PB7686 | MF3755 | MF4507 | ++ | + | + |
| PB8021 | MF3755 | MF3462 | + | + | + |
| PB8218 | MF3752 | MF4040 | ++ | +++ | + |
| PB8244 | MF3755 | MF4044 | ++ | + | ++ |
| PB8292 | MF3755 | MF4130 | ++ | + | ++ |
| PB8301 | MF4280 | MF4130 | + | ++ | + |
| PB8316 | MF3755 | MF4293 | ++ | + | + |
| PB8339 | MF3752 | MF4294 | + | + | + |
| PB8340 | MF3755 | MF4294 | ++ | + | ++ |
| PB8364 | MF3755 | MF4296 | ++ | + | ++ |
| PB8388 | MF3755 | MF4297 | ++ | +++ | ++ |
| PB8511 | MF3755 | MF4301 | ++ | +++ | ++ |
| PB8532 | MF3370 | MF4356 | + | +++ | + |
| PB8535 | MF3755 | MF4356 | ++ | +++ | ++ |
| PB8545 | MF4281 | MF4356 | + | +++ | + |
| PB8582 | MF3752 | MF4491 | + | − | + |
| PB8583 | MF3755 | MF4491 | ++ | +++ | ++ |
| PB8607 | MF3755 | MF4505 | ++ | − | ++ |
| PB8639 | MF3752 | MF4506 | + | +++ | + |
| PB8640 | MF3755 | MF4506 | ++ | +++ | ++ |
| PB8687 | MF3752 | MF4508 | + | ++ | + |
| PB8688 | MF3755 | MF4508 | + | − | + |

Bispecifics are indicated as PBXXXX and the different Fab arms with MGXXXX. The activity of the bispecifics in the individual assays is indicated as: − no effect; + inhibition of proliferation lower than positive control; ++ = inhibition of proliferation comparable to positive control antibody 5D5 Fab; +++ = Inhibition of proliferation higher than positive control antibody 5D5 Fab.

TABLE 5
Composition of the most potent EGFRxcMET bispecific antibodies
and their competition with reference antibodies.

| | | EGFR | | |
|---|---|---|---|---|
| | | blocking | | |
| | | compared | | Cross |
| | | to cetux | | reference |
| Bispecific | | (based on | cMET | antibody |
| antibody | EGFR arm | IC50) | arm | blocking |
| PB8535 | MF3755 | 100% | MF4356 | 5D5 |
| PB8640 | MF3755 | 100% | MF4506 | ND |
| PB8388 | MF3755 | 100% | MF4297 | 13.3.2 |
| PB8218 | MF3752 | 80% | MF4040 | 5D5 |
| PB8532 | MF3370 | 80% | MF4356 | 5D5 |

TABLE 6
Composition of bispecific antibodies. The pXX number indicates
the number of the production run and can be used to identify
whether the antibody was produced in an ADCC version or not.

| Bispecific antibody | cMET arm | EGFR arm | ADCC enhanced |
|---|---|---|---|
| Cetuximab | — | — | — |
| PB8532p05 | MF4356 | MF3370 | No |
| PB19474p01 | MF4356 | MF8232 | Yes |
| PB19475p01 | MF4356 | MF8233 | Yes |
| PB19476p01 | MF8230 | MF3370 | Yes |
| PB19477p01 | MF8230 | MF8232 | Yes |
| PB19478p01 | MF8230 | MF8233 | Yes |
| PB8532p06 | MF4356 | MF3370 | Yes |
| PB8532p04 | MF4356 | MF3370 | No |
| | HER-3 arm | EGFR arm | |
| PB4522p34 | MF3178 | MF4280 | Yes |
| PB4522p25 | MF3178 | MF4280 | No |
| | TT arm | TT arm | |
| PG1337p218 | MF1337 | MF1337 | No |

TABLE 7
Relevant characteristics of model LXFE2478.

| | | | Simple Western | | |
|---|---|---|---|---|---|
| | EGFR amino acid | Tumor | Size AUC | | |
| Model | change | type | EGFR | MET | HGF |
| LXFE2478 | M766_A767insASV | NSCLC | 1372205 | 54096 | 229544 |

AUC = area under the curve.

TABLE 8
Treatment plan for PDX model LXFE2478.

| | | | | | Dosing |
|---|---|---|---|---|---|
| | No. of | | | | Frequency |
| Group | mice | Treatment | Dose Level | Dose route | & Duration |
| 1 | 5 | Vehicle 1 | 10 mL/kg | PO | QD x 30 days |
| 2 | 5 | Antibody | 0.5 mg/kg | IP | QW x 5 weeks |
| 3 | 5 | PB19478 | 2.5 mg/kg | IP | QW x 5 weeks |
| 4 | 5 | PB19478 | 8 mg/kg | IP | QW x 5 weeks |
| 5 | 5 | PB19478 | 25 mg/kg | IP | QW x 5 weeks |

TABLE 9
Treatment plan for CDX model HCC827-ER1.

| | | | Dose | | Dosing |
|---|---|---|---|---|---|
| | | | level | Dosing | frequency & |
| Group | No. of mice | Treatment | (mg/kg) | route | duration |
| 1 | 8 | Vehicle | — | i.p. | BIW x 7 doses |
| 2 | 8 | PB19748 | 25 | i.p. | BIW x 7 doses |

BIW = twice a week, i.p. = intraperitoneal.

1. A bispecific antibody that comprises a first variable domain that can bind an extracellular part of human epidermal growth factor receptor (EGFR) and a second variable domain that can bind an extracellular part of human MET Proto-Oncogene, Receptor Tyrosine Kinase (cMET), wherein the first variable domain comprises a heavy chain variable region with a CDR1 sequence SYGIS; a CDR2 sequence WISAYX₁X₂NTNYAQKLQG and a CDR3 comprising the sequence X₃X₄X₅X₆HWWLX₇A, wherein X₁=N or S; X₂=A or G; X₃=D or G; X₄=R, S or Y; X₅=H, L or Y; X₆=D or W and X₇=D or G; with 0-5 amino acid insertions, deletions, substitutions, additions or a combination thereof at a position other than X₁-X₇ and wherein the second variable domain comprises a heavy chain variable region with the amino acid sequence of one of the sequences of SEQ ID NO: 1-23 with 0-10 preferably 0-5 amino acid insertions, deletions, substitutions, additions or a combination thereof, for use in a method of treatment of a cancer in a subject which has received prior treatment with i) a third-generation EGFR tyrosine kinase inhibitor or ii) a chemotherapy and a tyrosine kinase inhibitor or iii) a cMET tyrosine kinase inhibitor.

2. Method of treating a cancer in a subject which has received prior treatment with i) a third-generation EGFR tyrosine kinase inhibitor or ii) a chemotherapy and an EGFR tyrosine kinase inhibitor or iii) a cMET tyrosine kinase inhibitor, said treatment comprising administering to the subject an effective amount of a bispecific antibody that comprises a first variable domain that can bind an extracellular part of human epidermal growth factor receptor (EGFR) and a second variable domain that can bind an extracellular part of human MET Proto-Oncogene, Receptor Tyrosine Kinase (cMET), wherein the first variable domain comprises a heavy chain variable region with a CDR1 sequence SYGIS; a CDR2 sequence WISAYX₁X₂NTNYAQKLQG and a CDR3 comprising the sequence X₃X₄X₅X₆HWWLX₇A, wherein X₁=N or S; X₂=A or G; X₃=D or G; X₄=R, S or Y; X₅=H, L or Y; X₆=D or W and X₇=D or G; with 0-5 amino acid insertions, deletions, substitutions, additions or a combination thereof at a position other than X₁-X₇ and wherein the second variable domain comprises a heavy chain variable region with the amino acid sequence of one of the sequences of SEQ ID NO: 1-23 with 0-10 preferably 0-5 amino acid insertions, deletions, substitutions, additions or a combination thereof.

3. Use of a bispecific antibody that comprises a first variable domain that can bind an extracellular part of human epidermal growth factor receptor (EGFR) and a second variable domain that can bind (or binds) an extracellular part of human MET Proto-Oncogene, Receptor Tyrosine Kinase (cMET) wherein the first variable domain comprises a heavy chain variable region with a CDR1 sequence SYGIS; a CDR2 sequence WISAYX₁X₂NTNYAQKLQG and a CDR3 comprising the sequence X₃X₄X₅X₆HWWLX₇A, wherein X₁=N or S; X₂=A or G; X₃=D or G; X₄=R, S or Y; X₅=H, L or Y; X₆=D or W and X₇=D or G; with 0-5 amino acid insertions, deletions, substitutions, additions or a combination thereof at a position other than X₁-X₇ and wherein the second variable domain comprises a heavy chain variable region with the amino acid sequence of one of the sequences of SEQ ID NO: 1-23 with 0-10 preferably 0-5 amino acid insertions, deletions, substitutions, additions or a combination thereof, in the manufacture of a medicament for the treatment of a cancer in a subject which has received prior treatment with i) a third-generation EGFR tyrosine kinase inhibitor or ii) a chemotherapy and an EGFR tyrosine kinase inhibitor or iii) a cMET tyrosine kinase inhibitor.

4. The use or method according to any one of the preceding claims, wherein said subject or cancer which has received prior treatment according to i) is resistant to treatment with a third-generation EGFR tyrosine kinase inhibitor.

5. The use or method according to any one of claims 1-3, wherein said subject or cancer which has received prior treatment according to ii) is resistant to treatment with a first-, second and/or third-generation tyrosine kinase inhibitor.

6. The use or method according to any one of claims 1-3, wherein said subject or cancer which has received prior treatment according to iii) is resistant to treatment with a cMET tyrosine kinase inhibitor.

7. The use or method according to any one of the preceding claims, wherein the administration of said bispecific antibody and said EGFR tyrosine kinase inhibitor according to i) and iii) is administered as second line treatment, and according to ii) as third line treatment.

8. The use or method according to any one of the preceding claims, wherein said first-generation EGFR tyrosine kinase inhibitor comprises gefitinib, erlotinib or icotinib.

9. The use or method according to any one of the preceding claims, wherein said second-generation EGFR tyrosine kinase inhibitor comprises afatinib, dacomitinib, XL647, AP26113, CO-1686 or neratinib.

10. The use or method according to any one of the preceding claims, wherein said third-generation EGFR tyrosine kinase comprises Osimertinib, Lazertinib, Alflutinib, Rezivertinib, Rociletinib, Olmutinib, Almonertinib, Abivertinib, ASK120067, Befotertinib, SH-1028, Nazartinib (EGF816), Naquotinib (ASP8273), Mavelertinib (PF-0647775), Olafertinib (CK-101), Keynatinib, ES-072, preferably Osimertinib.

11. The use or method according to any one of the preceding claims, wherein said cMET tyrosine kinase inhibitor is or comprises capmatinib, tepotinib, crizotenib, cabozantinib, savolitinib, Glesatinib, Sitravatinib, BMS-777607, Merestinib, Tivantinib, Golvatinib, Foretinib, AMG-337 or BMS-794833

12. The use or method according to any one of the preceding claims, wherein said chemotherapy comprises platinum-based chemotherapy, cisplatin, carboplatin, oxaliplatin, paclitaxel, docetaxel, gemcitabine, vinorelbine, etoposide or pemetrexed, or any combination thereof, preferably a combination comprising cisplatin or carboplatin.

13. The use or method according to any one of the preceding claims, wherein said cancer is an EGFR positive and/or cMET positive cancer.

14. The use or method according to any one of the preceding claims, wherein said cancer comprises an EGFR and/or cMET aberration.

15. The use or method according to any one of the preceding claims, wherein said cancer comprises an activating EGFR mutation, an approved tyrosine kinase inhibitor resistance mutation, a tertiary tyrosine kinase inhibitor resistance mutation, a mutation that reduces binding of a third generation tyrosine kinase inhibitor to EGFR, an acquired tyrosine kinase inhibitor resistance mutation, an EGFR gene amplification, a cMET mutation or cMET aberration.

16. The use or method according to any one of the preceding claims, wherein said cancer comprises an exon 19 deletion mutation, preferably an in-frame exon 19 deletion, an exon 20 missense mutation or an exon 21 mutation.

17. The use or method according to any one of the preceding claims, wherein said cancer comprises an EGFR exon 20 mutation, preferably an exon 20 insertion mutation.

18. The use or method according to any one of the preceding claims, wherein said cancer comprises an acquired tyrosine kinase inhibitor resistance mutation such as a mutation which confers resistance to Osimertinib.

19. The use or method according to any one of the preceding claims, wherein said cancer comprises an exon 20 mutation selected from a near-loop insertion (positions 767-772), a far-loop insertion (positions 773-775), preferably V769_D770insASV, D770_N771insSVD, H773_V774insNPH, H773_V774insH, D770_N771insG, D770delinsGY, N771_P772insN, V774_C775insHV, D770_N771insGL, H773_V774insPH, A763_Y764insFQEA, D770_N771delinsEGN, D770_N771insGD, D770_N771insH, D770_N771insP, H773_V774insAH, H773_V774insGNPH, H773delinsSNPY, N771_P772insH, N771_P772insVDN, N771delinsGY, N771delinsKH, N771delinsRD, P772_H773delinsHNPY, P772_H773insGT, P772_H773insPNP, P772_H773insT, V769_D770insA, V769_D770insGG, V769_D770insGSV, V769_D770insGVV and V769_D770insMASV; or mutations T790M, L792X (e.g. L792H, C796X (e.g. G796R, G796S, G796D), C797X (e.g. C797S, C797G), L798I, or an in-frame exon 20 insertion, such as M766_A767insASV or H773-V774insNPH, Ins761 (EAFQ), Ins770 (ASV), Ins771 (G), Ins774 (NPH), M766_A7671 ns A, S768_V769InsSVA, P772_H773InsNS, D761_E762InsX1-7, A763_Y764InsX1-7, Y764_Y765 InsX1-7, M766_A767InsX1-7, A767_V768 InsX1-7, S768_V769 InsX1-7) V769_D770 InsX1-7) D770_N771 InsX1-7) N771_P772 InsX1-7) P772_H773 InsX1-7, H773_V774 InsX1-7, or V774_C775 InsX1-7.

20. The use or method according to any one of the preceding claims, wherein said cancer comprises a cMET aberration, such as a cMET amplification, cMET overexpression, increased signaling of the cMET pathway, a cMET gene amplification, increased HGF expression and/or increased cMET protein activity.

21. The use or method according to any one of the preceding claims, wherein said cancer comprises a cMET exon14 skipping mutation.

22. The use or method according to any one of the preceding claims, wherein said cancer is non-small cell lung cancer (NSCLC), head and neck cancer, gastric cancer, esophageal cancer, gastric/esophageal junction cancer, breast cancer, colon cancer, pancreatic cancer, ovarian cancer, colorectal cancer or bladder cancer.

23. The use or method according to any one of the preceding claims, wherein said cancer is advanced or metastatic cancer.

24. The use or method according to any one of the preceding claims, wherein said cancer or patient has progressed after having received said prior therapy for advanced or metastatic cancer.

25. The use or method according to any one of the preceding claims, wherein treatment comprises a diagnostic step for assessing whether said cancer is an EGFR positive and/or cMET positive cancer or assessing the presence of an EGFR and/or cMET aberration in said cancer.

26. The use or method according to any one of the preceding claims, wherein the subject is a human subject.

27. The use or method according to any one of the preceding claims, wherein the antibody is a human antibody.

28. The use or method according to any one of the preceding claims, wherein the antibody is ADCC enhanced.

29. The use or method according to any one of the preceding claims, wherein the antibody is an IgG1 format antibody having an anti-EGFR, anti-cMET stoichiometry of 1:1.

30. The use or method according to any one of the preceding claims, wherein the antibody has one variable domain that can bind EGFR and one variable domain that can bind cMET.

31. The use or method according to any one of the preceding claims, wherein the variable domain that can bind human EGFR can also bind cynomolgus and mouse EGFR.

32. The use or method according to any one of the preceding claims, wherein the variable domain that can bind human EGFR binds to domain III of human EGFR.

33. The use or method according to any one of the preceding claims, wherein the variable domain that can bind cMET blocks the binding of antibody 5D5 to cMET.

34. The use or method according to any one of the preceding claims, wherein the variable domain that can bind cMET blocks the binding of HGF to cMET.

35. The use or method according to any one of the preceding claims, wherein the amino acids at positions 405 and 409 in one CH3 domain are the same as the amino acids at the corresponding positions in the other CH3 domain (EU-numbering).

36. The use or method according to any one of the preceding claims, wherein
X₁=N; X₂=G; X₃=D; X₄═S; X₅=Y; X₆=W and X₇=G;
X₁=N; X₂=A; X₃=D; X₄═S; X₅=Y; X₆=W and X₇=G;
X₁═S; X₂=G; X₃=D; X₄═S; X₅=Y; X₆=W and X₇=G;
X₁=N; X₂=G; X₃=D; X₄=R; X₅=H; X₆=W and X₇=D;
X₁=N; X₂=A; X₃=D; X₄=R; X₅=H; X₆=W and X₇=D;
X₁═S; X₂=G; X₃=D; X₄=R; X₅=H; X₆=W and X₇=D;
X₁=N; X₂=G; X₃=G; X₄=Y; X₅=L; X₆=D and X₇=G;
X₁=N; X₂=A; X₃=G; X₄=Y; X₅=L; X₆=D and X₇=G; or
X₁═S; X₂=G; X₃=G; X₄=Y; X₅=L; X₆=D and X₇=G

37. The use or method according to any one of the preceding claims, wherein X₁=N; X₂=G; X₃=D; X₄=R; X₅=H; X₆=W and X₇=D; or X₁=N; X₂=A; X₃=D; X₄=R; X₅=H; X₆=W and X₇=D; or X₁═S; X₂=G; X₃=D; X₄=R; X₅=H; X₆=W and X₇=D.

38. The use or method according to any one of the preceding claims, wherein X₁=N; X₂=G; X₃=D; X₄=R; X₅=H; X₆=W and X₇=D; or X₁=N; X₂=A; X₃=D; X₄=R; X₅=H; X₆=W and X₇=D.

39. The use or method according to any one of the preceding claims, wherein the heavy chain variable region of the second variable domain comprises the amino acid sequence of one of the sequences of SEQ ID NO: 1-3; 7; 8; 10; 13; 15; 16; 17; 21; 22 or 23 with 0-10 preferably 0-5 amino acid insertions, deletions, substitutions, additions or a combination thereof.

40. The use or method according to any one of the preceding claims, wherein the heavy chain variable region of the second variable domain comprises the amino acid sequence of one of the sequences of SEQ ID NO: 2; 7; 8; 10; 13 or 23 with 0-10 preferably 0-5 amino acid insertions, deletions, substitutions, additions or a combination thereof.

41. The use or method according to any one of the preceding claims, wherein the first variable domain comprises a heavy chain variable region with a CDR1 sequence SYGIS; a CDR2 sequence WISAYNGNTNYAQKLQG and a CDR3 comprising the sequence DRHWHWWLDA and wherein the second variable domain comprises a heavy chain variable region with a CDR1 sequence SYSMN; a CDR2 sequence WINTYTGDPTYAQGFTG and a CDR3 sequence ETYYYDRGGYPFDP.

42. The use or method according to any one of the preceding claims, wherein the first variable domain comprises a heavy chain variable region with a CDR1 sequence SYGIS; a CDR2 sequence WISAYNANTNYAQKLQG and a CDR3 comprising the sequence DRHWHWWLDA and wherein the second variable domain comprises a heavy chain variable region with a CDR1 sequence TYSMN; a CDR2 sequence WINTYTGDPTYAQGFTG and a CDR3 comprising the sequence ETYFYDRGGYPFDP.

43. The use or method according to any one of the preceding claims, wherein the first and second variable domain comprise a common light chain, preferably a light chain variable domain of FIG. 4B.

44. The use or method according to any one of the preceding claims, which antibody inhibits HGF induced growth of an HGF-growth responsive cell.

45. The use or method according to any one of the preceding claims, which antibody inhibits EGF induced growth of an EGF-growth responsive cell.
